(19) United States
(12) Patent Application Publication
(10) Pub. No.: US 2012/0014975 A1
(43) Pub. Date: Jan. 19, 2012

(54) MODIFIED SINGLE DOMAIN ANTIGEN BINDING MOLECULES AND USES THEREOF

(75) Inventors: Martin Hegen, Brookline, MA (US); Stephane Hubert Olland, Arlington, MA (US); Yulia Vugmeyster, North Reading, MA (US); Xin Xu, Andover, MA (US)

(73) Assignee: Wyeth LLC, Madison, NJ (US)

(21) Appl. No.: 13/182,560

(22) Filed: Jul. 14, 2011

Related U.S. Application Data
(60) Provisional application No. 61/365,307, filed on Jul. 16, 2010.

(51) Int. Cl. A61K 47/48; G01N 33/53; A61B 19/00; A61P 29/00; A61P 37/06; G01N 33/566; C07K 17/08; A61K 49/00

(52) U.S. Cl. 424/179.1; 530/391.1; 435/7.21; 800/3; 436/501; 206/438

(57) ABSTRACT

The invention relates to modified single domain antigen binding molecules, e.g., SDAB molecules, in particular TNFα-binding SDAB molecules. Method of preparing, and using the modified single domain antigen binding molecules described herein, to treat, e.g., TNFα-associated disorders, are also disclosed.

This application claims priority to U.S. Provisional Application No. 61/365,307, filed Jul. 16, 2010, the contents of which are incorporated by reference.

### BACKGROUND

Tumor necrosis factor alpha (TNFα) is a secreted and membrane-bound pro-inflammatory cytokine produced mainly by macrophages and monocytes. The synthesis of TNFα is upregulated in various chronic autoimmune inflammatory diseases, such as rheumatoid arthritis, ulcerative colitis, Crohn's Disease, and others. TNFα is expressed as a trimeric transmembrane protein that can be proteolytically cleaved to release its soluble form by TNFα converting enzyme (TACE). Both forms of TNFα interact with TNF receptor (TNFR) 1 and TNFR2.

### SUMMARY

The invention relates to modified single domain antigen binding molecules (also referred to herein as “SDAB molecules.” The modified SDAB molecule can include one or more single antigen binding domains that interact with, e.g., bind to, one or more targets. In one embodiment, one or more of the single antigen binding domains of the modified SDAB molecule bind to tumor necrosis factor-α (TNFα). The SDAB molecule can be modified to increase its biological properties in vivo. For example, the SDAB molecule can be modified to improve one or more of: increased half life; reduced immunogenicity; or improve at least one pharmacokinetic/pharmacodynamic (PK/PD) parameter, compared to the unmodified SDAB molecule. In one embodiment, the modified SDAB molecule includes one or more polymer molecules, such as poly(ethyleneglycol) (PEG) or a derivative thereof. The modified SDAB molecules are useful, e.g., for administration to a subject, e.g., a human. Methods of preparing, and using, the modified SDAB molecules, to treat or prevent, e.g., TNFα-associated disorders, are also disclosed.

Accordingly, in one aspect, the invention features a modified SDAB molecule that includes: (i) one or more single antigen binding domains that interact with, e.g., bind to, one or more targets (e.g., TNFα); (ii) a linker (e.g., a non-peptidic linker and/or a peptidic linker); and (iii) one or more polymer molecules, such as poly(ethyleneglycol) (PEG) or a derivative thereof. In one embodiment, the linker of the SDAB molecule is a non-peptidic linker. In certain embodiments, the SDAB molecules can be modified by associating, e.g., covalently or non-covalently, to a second moiety, e.g., a polymer molecule. For example, the SDAB molecule can be covalently attached to a suitable pharmacologically acceptable polymer, such as poly(ethyleneglycol) (PEG) or a derivative thereof (such as methoxypoly(ethyleneglycol) or mPEG).

In one embodiment, the modified SDAB molecule includes one or more single binding domains. For example, the SDAB molecule can comprise, or consist of, a polypeptide, e.g., a single chain polypeptide, comprising at least one immunoglobulin variable domain (including one, two, or three complementarity determining regions (CDRs)). Examples of SDAB molecules include molecules naturally devoid of light chains (e.g., VHH, nanobodies, or camelid derived antibodies). Such SDAB molecules can be derived or obtained from camelids such as camel, llama, dromedary, alpaca, and guanaco. In other embodiments, the SDAB molecule may include one or more single domain molecules including, but not limited to, other naturally-occurring single domain molecules (e.g., shark single domain polypeptides (IgNAR)), and single domain scaffolds (e.g., fibronectin scaffolds).

In another embodiment, the modified SDAB molecule is a single chain polypeptide comprised of one or more single antigen binding domains. The SDAB molecules can bind to the same target, e.g., at the same or different epitopes, or different targets. The single antigen binding domains of the SDAB molecule can have the same or different amino acid sequence. In some embodiments, the SDAB molecule is monovalent or multivalent (e.g., bivalent, trivalent, or tetravalent). In other embodiments, the SDAB molecule is monospecific or multispecific (e.g., bispecific, trispecific, or tetraspecific). The SDAB molecule may comprise one or more single antigen binding domains that are recombinant, CDR-grafted, humanized, camelized, de-immunized, and/or in vitro generated (e.g., selected by phage display). For example, the SDAB molecule can be a single chain fusion polypeptide comprising one, two, three, four, or more single antigen binding domains that bind to one or more target antigens. Typically, the target antigen is a mammalian, e.g., a human, protein. In one embodiment, the target antigen is TNFα, e.g., human TNFα.

In one exemplary embodiment, the modified SDAB molecule is a bivalent molecule composed of a single chain polypeptide fusion of two single antigen binding domains (e.g., two camelid variable regions) that bind to a target antigen, e.g., TNFα. The single antigen binding domains of the modified SDAB molecule can be arranged in the following order from N- to C-terminus: TNFα-binding single antigen binding domain—(optionally a linking group, e.g., a peptidic linker)—TNFα-binding single antigen binding domain—one or more polymer molecules. In one embodiment, the single antigen binding domains bind to the same epitope on the target antigen (for example, the same or different single antigen binding domains are used). In other embodiments, the single antigen binding domains of the SDAB molecule bind to different epitopes on the same or different targets. It will be appreciated that any order or combination of two, three, four, or more single antigen binding domains against one or more targets is encompassed by the present invention.

In other embodiments, two, three, four or more of the single domain molecules of the modified SDAB molecules are associated (e.g., fused), with or without a linking group, as a genetic or a polypeptide fusion. The linking group can be any linking group apparent to those of skill in the art. For instance, the linking group can be a biocompatible polymer with a length of 1 to 100 atoms. The linking group can be a peptidic or non-peptidic linker. In one embodiment, the linking group is a peptidic linker, e.g., it includes or consists of polyglycine, polyserine, polylysine, polyglutamate, polyisoleucine, or polyarginine residues, or a combination thereof. For example, the polyglycine or polyserine linking groups can include at least five, seven, eight, nine, ten, twelve, fifteen, twenty, thirty, thirty-five, and forty glycine and serine residues. Exemplary linking groups that can be used include Gly-Ser repeats, for example, (Gly)₃-Ser (SEQ ID NO:7) or (Gly)₄-Ser (SEQ ID NO: 8) repeats of at least one, two, three, four, five, six, seven or more repeats. In some embodiments, the linking group has the following sequences: (Gly)₄-Ser-(Gly)₃-Ser (SEQ ID NO: 9) or ((Gly)₄-Ser)ₙ (SEQ ID NO: 10), where n is 4, 5, or 6. In one embodiment, the linking group includes the following sequence: ((Gly)₄-Ser)ₙ (SEQ ID NO:10), where n=6. The modified SDAB molecule can additionally include a linking group at the C-terminus of the single antigen binding domain (e.g., referred to herein as “C-terminal linking group”) to facilitate attachment of the SDAB to another moiety (e.g., a carrier molecule, a non-peptidic linker or moiety). Any of the linking groups described herein can be used as a C-terminal linking group. In one embodiment, one or more Gly-Ser repeats are used; for example, one or more repeats of (Gly)₃-Ser or (Gly)₄-Ser (SEQ ID NO: 8) are used.

In one embodiment, the modified SDAB molecule (referred to herein as “SDAB-01”) comprises, or consists of, the amino acid sequence shown in FIG. 1 (SEQ ID NO:1), or an amino acid sequence substantially identical thereto (e.g., an amino acid sequence at least 85%, 90%, 95% or more identical to, or having up to 20, 15, 10, 5, 4, 3, 2, 1 amino acid changes (e.g., deletions, insertions or substitutions (e.g., conservative substitutions)) relative to the amino acid sequence shown in FIG. 1). The nucleotide sequence encoding the two single antigen binding domains of SEQ ID NO:1 is provided as SEQ ID NO:6 (see Table 12). In other embodiments, the modified SDAB comprises or consists of an amino acid sequence encoded by SEQ ID NO:6, or a nucleotide sequence substantially identical thereto (e.g., a nucleotide sequence at least 85%, 90%, 95% or more identical to, or having up to 60, 45, 30, 15, 12, 9, 6, 3 nucleotide changes relative to the amino acid sequence of SEQ ID NO:6).

Examples of additional single domain molecules include, but are not limited to, the amino acid sequences disclosed in Table 19 of WO 2006/122786 (incorporated by reference herein), and in Table 11 below.

In certain embodiments, at least one of the single antigen binding domains of the modified SDAB molecule that binds to TNFα includes one, two, or three CDRs having the amino acid sequence: DYWMY (SEQ ID NO:2) (CDR1), EINTNGLITKYPDSVKG (SEQ ID NO:3) (CDR2) and/or SPSGFN (SEQ ID NO:4) (CDR3), or having a CDR that differs by fewer than 3, 2, or 1 amino acid substitutions (e.g., conservative substitutions) from one of said CDRs. In other embodiments, the single antigen binding domain comprises a variable region having the amino acid sequence from about amino acids 1 to 115 of FIG. 1, or an amino acid sequence substantially identical thereto (e.g., an amino acid sequence at least 85%, 90%, 95% or more identical to, or having up to 20, 15, 10, 5, 4, 3, 2, 1 amino acid changes (e.g., deletions, insertions or substitutions (e.g., conservative substitutions) relative to the amino acid sequence shown in FIG. 1). In some embodiments, the TNFα-binding SDAB molecule has one or more biological activities of the TNFα-binding single domain antibody molecule shown in FIG. 1. For example, the TNFα-binding SDAB molecule binds to the same or a similar epitope as the epitope recognized by the TNFα-binding single domain molecule shown in FIG. 1 (e.g., binds to TNFα in its trimeric form; binds to the TNFα site contacting the TNF receptor; binds to an epitope in the TNFα trimer comprising Gln at position 88 and Lys at position 90 on the first TNF monomer (monomer A), and Glu at position 146 on the second TNF monomer (monomer B), or an epitope as disclosed in WO 06/122786). In other embodiments, the TNFα-binding SDAB molecule binds to the N-terminus of TNFα. In other embodiment, the TNFα-binding SDAB molecule has an activity (e.g., binding affinity, dissociation constant, binding specificity, TNFα-inhibitory activity) similar to any of the TNFα-binding single domain molecule disclosed in WO 06/122786.

In other embodiments, the TNFα-binding SDAB molecule comprises one or more of the SDAB molecules disclosed in Table 11, also disclosed in WO 2006/122786, incorporated by reference. For example, the TNFα-binding SDAB molecule can be a monovalent, bivalent, or trivalent TNFα-binding SDAB molecule disclosed in Table 9 of WO 2006/122786. Exemplary TNFα-binding SDAB molecules include, but are not limited to, TNF1, TNF2, TNF3, and humanized forms thereof (e.g., TNF29, TNF30, TNF31, TNF32, TNF33). Additional examples of monovalent TNFα-binding SDAB molecules are disclosed in Table 8 of WO 2006/122786. Exemplary bivalent TNFα-binding SDAB molecules include, but are not limited to, TNF55 and TNF56, which comprise two TNF30 SDAB molecules linked via a peptide linker to form a single fusion polypeptide (disclosed in WO 2006/122786). Additional examples of bivalent TNFα-binding SDAB molecules are disclosed in Table 11 herein, or in Table 19 of WO 2006/122786 as TNF4, TNF5, TNF6, TNF7, TNF8).

In certain embodiments, the SDAB molecule is modified to include one or more polymer molecules, such as poly(ethyleneglycol) (PEG) or a derivative thereof. The PEG molecule (e.g., a PEG monomer, polymer or a derivative thereof) can be linear or branched. In one embodiment, the SDAB is attached to one or more PEG molecules via a linker moiety (e.g., a non-peptidic linker).

In some embodiments, the linker is a non-peptidic linker. In one embodiment, the linker is represented by formula (I):

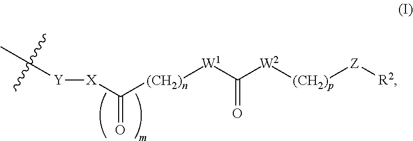

### Wherein

W¹ and W² are each independently selected from a bond or NR¹;
Y is a bond, C₁₋₄ alkylene substituted with 0-2 occurrences of R^{a} or a pyrrolidine-2,5-dione;
X is O, a bond or is absent;
Z is absent, O, NR³, S or a bond;
R¹ and R³ are each independently hydrogen or C₁₋₆ alkyl;
R² is absent or is one or more polymer moieties;
R^{a} is selected from hydroxyl, C₁₋₄ alkyl, or C₁₋₄ alkoxy;
m is 0 or 1;
n is 0, 1, 2 or 3; and
p is 0, 1, 2, 3, or 4.

In some embodiments, the one or more polymer moieties of the SDAB molecule (e.g., R² of Formula (I)) include a poly(ethyleneglycol) (PEG) molecule (e.g., a PEG monomer, polymer or a derivative thereof). In some embodiments, the PEG molecule is a methoxypoly(ethyleneglycol) (mPEG) monomer, polymer or a derivative thereof.

In some embodiments, the PEG molecule is branched. In some embodiments, the PEG molecule is selected from a moiety of formulas (a)-(h);

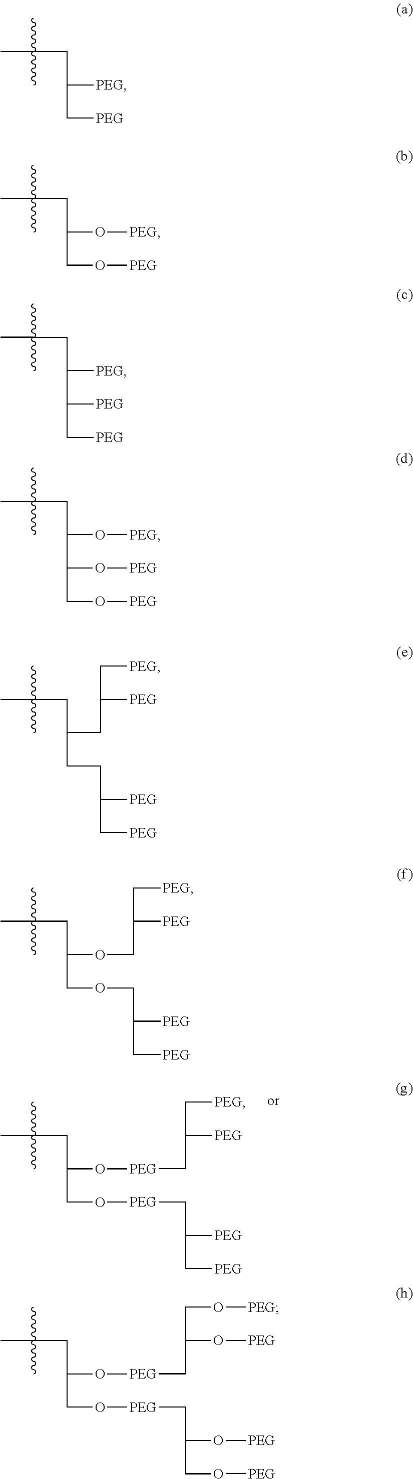

wherein each PEG molecule is independently a PEG monomer, polymer, or a derivative thereof. In some embodiments, each PEG molecule is an mPEG monomer, polymer, or a derivative thereof.

In some embodiments, Y is a bond. In some embodiments, Y is pyrrolidine-2,5-dione. In some embodiments, Y is C₁₋₄ alkylene substituted with 0-2 occurrences of R^{a}. In some embodiments, Y is C₁₋₄ alkylene substituted with 1 occurrence of R^{a}. In some embodiments, Y is methylene substituted with 1 occurrence of R^{a}. In some embodiments, R^{a} is hydroxyl.

In some embodiments, X is a bond. In some embodiments, X is oxygen (O). In some embodiments, X is absent.

In some embodiments, R² is (a).

In some embodiments, R² is (g).

In some embodiments, W¹ is a bond. In some embodiments, W¹ is NR¹.

In some embodiments, W² is a bond. In some embodiments, W² is NR¹.

In some embodiments, R¹ is hydrogen.

In some embodiments, Z is O, S or a bond.

In some embodiments, Z is O.

In some embodiments, R³ is hydrogen.

In some embodiments, m is 0. In some embodiments, m is 1.

In some embodiments, n is 0. In some embodiments, n is 2. In some embodiments, n is 3.

In some embodiments, p is 0. In some embodiments, p is 3.

In some embodiments, each PEG molecule is independently a PEG monomer, polymer, or a derivative thereof. In some embodiments, each PEG molecule is a methoxy PEG derivative (mPEG) monomer, polymer, or a derivative thereof. In some embodiments, each PEG molecule independently has a molecular weight between 1 KDa and 100 KDa. In some embodiments, each PEG molecule independently has a molecular weight between 10 KDa and 50 KDa. In some embodiments, each PEG molecule independently has a molecular weight of 40 KDa. In some embodiments, each PEG molecule independently has a molecular weight of between 15 KDa and 35 KDa. In some embodiments, each PEG molecule independently has a molecular weight of 30 KDa. In some embodiments, each PEG molecule independently has a molecular weight of 20 KDa. In some embodiments, each PEG molecule independently has a molecular weight of 17.5 KDa. In some embodiments, each PEG molecule independently has a molecular weight of 12.5 KDa. In some embodiments, each PEG molecule independently has a molecular weight of 10 KDa. In some embodiments, each PEG molecule has a molecular weight of 7.5 KDa. In some embodiments, each PEG molecule independently has a molecular weight of 5 KDa.

In some embodiments, the modified SDAB molecule includes a linker of formula (I) linked to a PEG molecule and has a structure selected from:

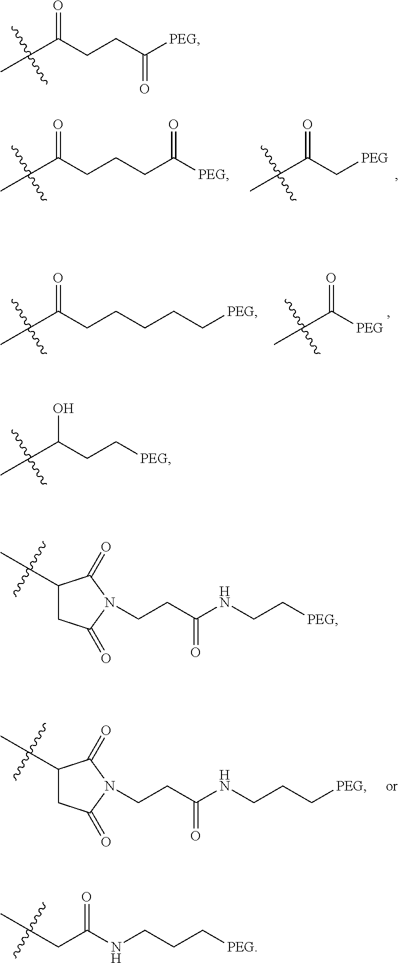

In some embodiments, the modified SDAB molecule includes a linker of formula (I) linked to a PEG molecule and has a structure selected from:

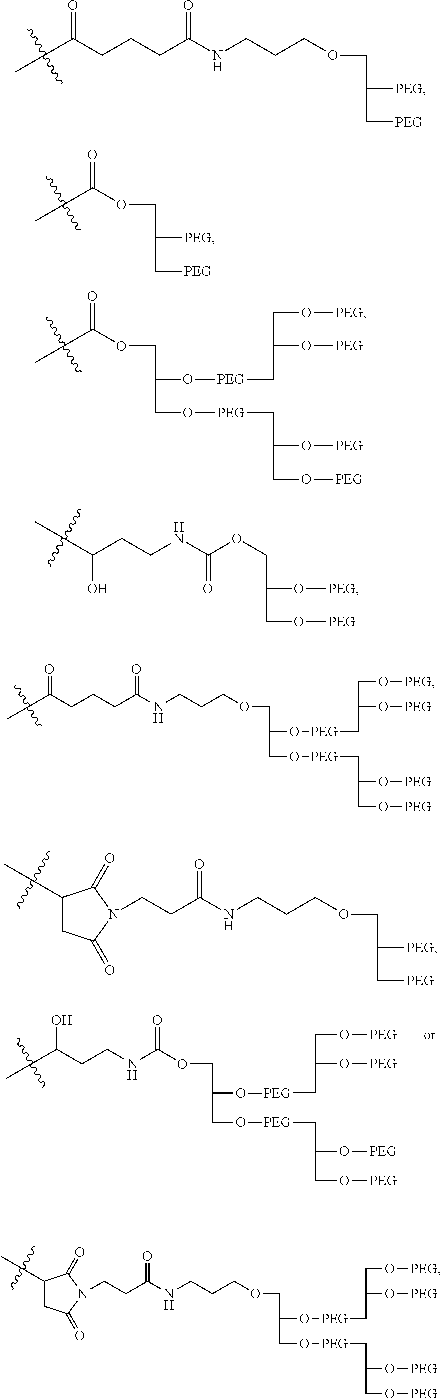

wherein each PEG molecule is independently a PEG monomer, polymer, or a derivative thereof. In some embodiments, each PEG molecule is an mPEG monomer, polymer, or a derivative thereof.

In some embodiments, the linker of formula (I) is linked to a PEG molecule represented by the following formula:

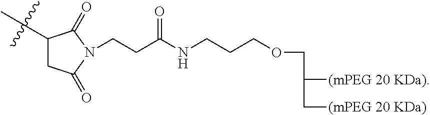

In some embodiments, the linker of formula (I) is linked to a PEG molecule represented by the following formula:

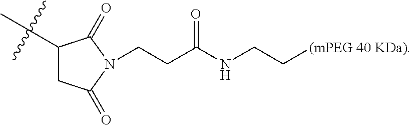

In some embodiments, the linker of formula (I) is linked to a PEG molecule represented by the following formula:

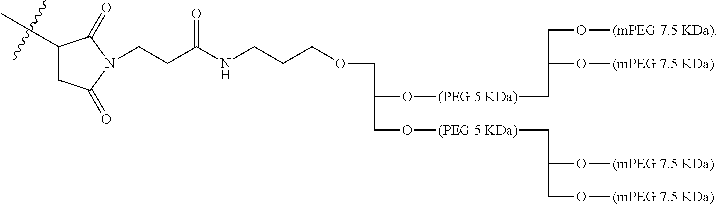

The linker-PEG molecule can be associated with (e.g., coupled to) the SDAB molecule, thereby forming a modified SDAB molecule. The single domain molecules of the SDAB molecule can be arranged in the following order from N- to C-terminus: TNFα-binding single domain molecule—TNFα-binding single domain molecule—PEG molecule (e.g., branched PEG molecule). In one embodiment, the modified SDAB molecule is represented by the following formula:

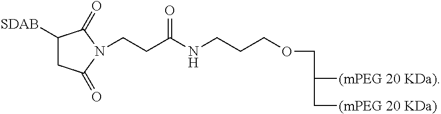

In one embodiment, the modified SDAB molecule is represented by the following formula:

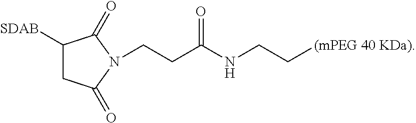

In one embodiment, the modified SDAB molecule is represented by the following formula:

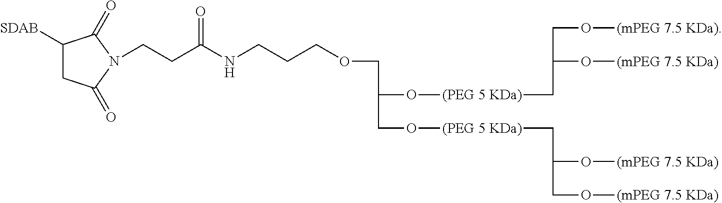

One exemplary embodiment of the modified SDAB molecule is represented by the following formula:

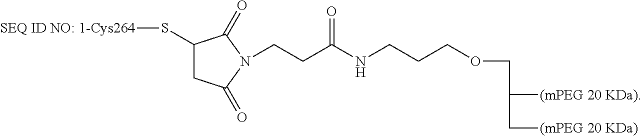

The reactive group of the SDAB molecule is generally attached via a nucleophilic moiety attached to the SDAB molecule. In some embodiments, the nucleophilic moiety is a sulfur (e.g., a sulfur from a cysteine residue). In other embodiments, the nucleophilic moiety is a nitrogen (e.g., from a terminal alpha-amino group or a nitrogen containing amino acid side chain (e.g., an ε-amino group from a lysine chain). In other embodiments, the nucleophilic moiety is a C-terminal group. The reactive group of the SDAB molecule is generally attached via an electrophilic moiety attached to the linker. In some embodiments, the electrophilic moiety is a carbonyl group (e.g., an activated ester or an aldehyde). In some embodiments, the electrophilic moiety is a maleimide group.

In another aspect, the invention features a method of making a modified SDAB molecule described herein. The method includes: providing an SDAB molecule (e.g., obtaining an SDAB molecule from a cell culture (e.g., a recombinant cell culture)); and contacting the SDAB molecule (e.g., the single antigen binding domain, or a linker (e.g., a peptidic linker attached thereto) with a linker moiety of formula (I) wherein Y, X, W¹, W², Z, R¹, R², R³, m, n and p are as described above, under conditions where at least one chemical bond is formed.

In some embodiments, Y is a bond. In some embodiments, Y is pyrrolidine-2,5-dione. In some embodiments, Y is C₁₋₄ alkylene substituted with 0-2 occurrences of R^{a}. In some embodiments, Y is C₁₋₄ alkylene substituted with 1 occurrence of R^{a}. In some embodiments, Y is methylene substituted with 1 occurrence of R^{a}. In some embodiments, R^{a} is hydroxyl.

In some embodiments, X is a bond. In some embodiments, X is oxygen (O). In some embodiments, X is absent.

In some embodiments, R² is (a).

In some embodiments, R² is (g).

In some embodiments, W¹ is a bond. In some embodiments, W¹ is NR¹.

In some embodiments, W² is a bond. In some embodiments, W² is NR¹.

In some embodiments, R¹ is hydrogen.

In some embodiments, Z is O, S or a bond.

In some embodiments, Z is O.

In some embodiments, R³ is hydrogen.

In some embodiments, m is 0. In some embodiments, m is 1.

In some embodiments, n is 0. In some embodiments, n is 2. In some embodiments, n is 3.

In some embodiments, p is 0. In some embodiments, p is 3.

In some embodiments, the SDAB molecule is linked via a cysteine residue.

In some embodiments, the SDAB molecule is reduced prior to treatment with a linker moiety of formula (I). In some embodiments, the SDAB molecule is reduced to eliminate disulfide bridges formed between cysteine residues.

In some embodiments, the linker of formula (I) is linked to a PEG molecule represented by the following formula:

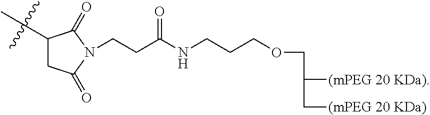

In one embodiment, the modified SDAB molecule is represented by the following formula:

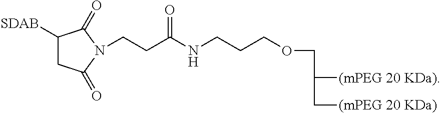

In another aspect, the invention features a composition, e.g., a pharmaceutical composition, that includes a modified SDAB molecule as described herein and a pharmaceutically acceptable carrier. The compositions can also include a second agent, e.g., a second therapeutically or pharmacologically active agent that is useful in treating a TNFα associated disorder, e.g., inflammatory or autoimmune disorders, including, but not limited to, rheumatoid arthritis (RA) (e.g., moderate to severe rheumatoid arthritis), arthritic conditions (e.g., psoriatic arthritis, polyarticular juvenile idiopathic arthritis (JIA), ankylosing spondylitis (AS), psoriasis, ulcerative colitis, Crohn's disease, inflammatory bowel disease, and/or multiple sclerosis.

In yet another aspect, the invention features a method of ameliorating an inflammatory or autoimmune condition in a subject. For example, a method of treating or preventing in a subject (e.g., a human subject) a TNFα associated disorder, e.g., inflammatory or autoimmune disorders. Examples of TNFα associated disorders include, but are not limited to, rheumatoid arthritis (RA) (e.g., moderate to severe rheumatoid arthritis), arthritic conditions (e.g., psoriatic arthritis, polyarticular juvenile idiopathic arthritis (JIA)), ankylosing spondylitis (AS), psoriasis, ulcerative colitis, Crohn's disease, inflammatory bowel disease, and/or multiple sclerosis. The method includes administering to the subject, e.g., a human patient, a TNFα-binding modified SDAB molecule as described herein, alone or in combination with a second therapeutically or pharmacologically active agent that is useful in treating a TNFα associated disorder, in an amount such that one or more of the symptoms of the TNFα associated disorder are reduced.

In one embodiment, the modified SDAB molecules (e.g., the compositions containing the modified SDAB molecules) described herein are suitable for administration to a subject, e.g., a human subject (e.g., a patient having a TNFα associated disorder). The SDAB molecules can be administered to the subject by injection (e.g., subcutaneous, intravascular, intramuscular or intraperitoneal) or by inhalation.

In certain embodiments, the modified SDAB molecule and the second agent are administered in combination, e.g., simultaneously or sequentially. In one embodiment, the modified SDAB molecule and the second agent are administered in the same composition, e.g., a pharmaceutical composition as described herein. In one embodiment, the second agent is an anti-TNFα antibody molecule or TNFα binding fragment thereof, wherein the second TNFα antibody binds to a different epitope than the TNFα-binding modified SDAB molecule described herein. Other non-limiting examples of second agents that can be co-administered or co-formulated with the TNFα-binding modified SDAB molecule include, but are not limited to, a cytokine inhibitor, a growth factor inhibitor, an immunosuppressant, an anti-inflammatory agent, a metabolic inhibitor, an enzyme inhibitor, a cytotoxic agent, and a cytostatic agent. In one embodiment, the additional agent is a standard treatment for arthritis, including, but not limited to, non-steroidal anti-inflammatory agents (NSAIDs); corticosteroids, including prednisolone, prednisone, cortisone, and triamcinolone; and disease modifying anti-rheumatic drugs (DMARDs), such as methotrexate, hydroxychloroquine (Plaquenil) and sulfasalazine, leflunomide (Arava®), tumor necrosis factor inhibitors, including etanercept (Enbrel®), infliximab (Remicade®) (with or without methotrexate), and adalimumab (Humira®), anti-CD20 antibody (e.g., Rituxan®), soluble interleukin-1 receptor, such as anakinra (Kineret®), gold, minocycline (Minocin®), penicillamine, and cytotoxic agents, including azathioprine, cyclophosphamide, and cyclosporine. Such combination therapies may advantageously utilize lower dosages of the administered therapeutic agents, thus avoiding possible toxicities or complications associated with the various monotherapies. Alternative combination of excipients and/or second therapeutic agents can be identified and tested following the guidance provided herein.

In another aspect, the invention features a method of evaluating a modified SDAB molecule, e.g., a modified SDAB molecule as described herein. The method includes administering a modified SDAB molecule as described herein to a subject, e.g., a human subject (e.g., a patient having a TNFα-associated disorder); and evaluating one or more pharmacokinetic/pharmacodynamic (PK/PD) parameters of the modified SDAB molecule. The SDAB molecules can be administered to the subject by injection (e.g., subcutaneous, intravascular, intramuscular or intraperitoneal) or by inhalation.

In a related aspect, the invention features a method of evaluating or selecting a modified SDAB molecule (e.g., a modified TNFα-binding SDAB molecule described herein). The method includes:

providing a test value, e.g., a mean test value, for at least one PK/PD parameter of SDAB molecule in a subject, e.g., a human or animal subject; and

comparing the test value, e.g., mean test value, provided with at least one reference value, to thereby evaluate or select the SDAB molecule.

In some embodiments, the step of providing a test value includes obtaining a sample of the SDAB molecule, e.g., a sample batch of an antibody cell culture and/or after modification of the SDAB molecule, and testing for at least one of the pharmacokinetic parameters described herein. Methods disclosed herein can be useful from a process standpoint, e.g., to monitor or ensure batch-to-batch consistency or quality.

In certain embodiments, the method of evaluating the modified SDAB molecule further includes: providing a sample, e.g., a sample containing a modified SDAB molecule; and testing the sample in a capture detection assay, e.g., a protein detection or whole molecule detection assay described herein in Example 11b. In one embodiment, the sample is contacted with a target immobilized to a solid support (e.g., a biotinylated target molecule associated with bound streptavidin); the bound SDAB-target molecule complex is detected using reagent, e.g., an antibody, that binds to the protein moiety of the modified SDAB molecule. In such assay format, the protein moiety of the modified SDAB molecule is detected. In other embodiments, the sample is contacted with a target immobilized to a solid support (e.g., a biotinylated target molecule associated with bound streptavidin); the bound SDAB-target molecule complex is detected using reagent, e.g., an antibody, that binds to the polymer, e.g., PEG, moiety of the modified SDAB molecule. In such embodiments, the polymer (e.g., PEGylated) moiety of the SDAB molecule is detected. Preferably, detection of the polymer (e.g., PEG) moiety captures the entire modified SDAB-polymer conjugate, since the unconjugated SDAB molecule is not detected.

The PK/PD parameter evaluated by the present methods can be chosen from one or more of: an in vivo concentration of the modified SDAB molecule (e.g., a concentration in blood, serum, plasma and/or tissue); clearance of the modified SDAB molecule (CL); volume distribution of the modified SDAB molecule (V_{dss} or Vc); half-life of the modified SDAB molecule (t_{1/2}); bioavailability of the modified SDAB molecule; maximum blood, serum plasma, or tissue concentration of the modified SDAB molecule; exposure (AUC=area under the concentration-time curve) of the modified SDAB molecule; tissue-to-serum, tissue-to-plasma, or tissue-to-blood AUC or concentration ratio of the modified SDAB molecule; urine concentrations of intact or degradation produce of the modified SDAB molecule; or free, bound, and total target concentrations in serum, plasma, or tissues.

In one embodiment, the one or more PK/PD parameters are evaluated at one, two, or more pre-determined time intervals after administration of the modified SDAB molecule to the subject. In one embodiment, at least one PK/PD parameter of the modified SDAB molecule is altered, e.g., improved, compared to a reference standard, e.g., the unmodified SDAB molecule. For example, the modified SDAB molecule has one or more of an increased half-life and/or bioavailability; different tissue distribution (e.g., localized to a different tissue or organ (e.g., the small or large intestine), compared to the unmodified SDAB molecule. In certain embodiments, the PK/PD parameters are used to provide a measure of efficacy value or suitability for treatment. Other measures of efficacy including, but not limited, amelioration of one or more symptoms, improved quality of life, decrease in inflammatory markers, can additionally be performed as part of the efficacy evaluation.

In some embodiments, the one or more PK/PD parameters, efficacy value, or an indication of whether the preselected efficacy standard is met, is/are recorded or memorialized, e.g., in a computer readable medium. Such values or indications of meeting pre-selected efficacy standard can be listed on the product insert, a compendium (e.g., the U.S. Pharmacopeia), or any other materials, e.g., labeling that may be distributed, e.g., for commercial use, or for submission to a U.S. or foreign regulatory agency.

In another aspect, the invention features a method for detection, or a capture detection assay, e.g., a protein detection or whole molecule detection assay described herein in Example 11b. The method or assay includes: providing a sample containing a modified SDAB molecule (e.g., obtaining sample obtained from a subject at after administration of an SDAB molecule); contacting the sample with a target (e.g., TNFα) immobilized to a solid support (e.g., a biotinylated target molecule associated with bound streptavidin); detecting the bound SDAB-target complex using reagent, e.g., an antibody, that binds to the protein or polymer (e.g., PEG) moiety of the modified SDAB molecule. In the assay format where the reagent binds to the protein moiety of the SDAB molecule, the protein moiety of the modified SDAB molecule is detected. In assay formats where the reagent binds to the PEG moiety of the modified SDAB molecule, the polymer (e.g., PEGylated) moiety of the SDAB molecule is detected. Preferably, detection of the PEG moiety captures the entire modified SDAB-polymer conjugate, since the unconjugated SDAB molecule is not detected.

In another aspect, the invention features a kit or an article of manufacture that includes a device, a syringe, or a vial containing the SDAB molecules or compositions described herein. The kit or article may, optionally, include instructions for use. In certain embodiments, the syringe or a vial is composed of glass, plastic, or a polymeric material, such as cyclic olefin polymer or copolymer. In other embodiments, the formulation can be present in an injectable device (e.g., an injectable syringe, e.g., a prefilled injectable syringe). The syringe may be adapted for individual administration, e.g., as a single vial system including an autoinjector (e.g., a pen-injector device), and/or instructions for use. In one embodiment, the injectable device is a prefilled pen or other suitable autoinjectable device, optionally with instruction for use and administration.

In certain embodiments, the kit or article of manufacture (e.g., the prefilled pen or syringe with a single or multiple dose unit) is provided to a subject, e.g., a patient or a healthcare provider, prepackaged with instructions for administration (e.g., self-administration) by injection (e.g., subcutaneous, intravascular, intramuscular, intraarticular, or intraperitoneal).

In other embodiments, the invention features a device for nasal, transdermal, intravenous administration of the formulations described herein is provided. For example, a transdermal patch for administration of the formulations described herein is provided. In yet other cases, an intravenous bag for administration of the formulations described herein is provided. In some embodiments, the intravenous bag is provided with normal saline or 5% dextrose.

In another aspect, the invention features a method of instructing a patient (e.g., a human patient) in need of a modified SDAB molecule, e.g., a TNFα SDAB molecule, how to administer the modified SDAB molecule or composition described herein. The method includes: (i) providing the patient with at least one unit dose of the SDAB molecule described herein; and (ii) instructing the patient to self-administer the at least one unit dose, e.g., by injection (e.g., subcutaneous, intravascular, intramuscular or intraperitoneal). In one embodiment, the patient has a TNFα associated disorder, e.g., inflammatory or autoimmune disorders as described herein.

In another aspect, the invention features a method of instructing a recipient on the administration of a modified SDAB molecule described herein. The method includes instructing the recipient (e.g., an end user, patient, physician, retail or wholesale pharmacy, distributor, or pharmacy department at a hospital, nursing home clinic or HMO) how the formulation should be administered to a patient.

In another aspect, a method of distributing a modified SDAB molecule described herein is provided. The method includes providing a recipient (e.g., an end user, patient, physician, retail or wholesale pharmacy, distributor, or pharmacy department at a hospital, nursing home clinic or HMO) with a package containing sufficient unit dosages of the SDAB molecule, to treat a patient for at least 6, 12, 24, or 36 months.

In another aspect, the invention features a method or process of evaluating the quality of a package or lot of packages (e.g., to determine if it has expired) of a formulation described herein containing a modified SDAB molecule described herein The method includes evaluating whether the package has expired. The expiration date is at least 6, 12, 24, 36, or 48 months, e.g., greater than 24 or 36 months, from a preselected event, such as manufacturing, assaying, or packaging. In some embodiments, a decision or step is taken as a result of the analysis, e.g., the SDAB molecule in the package is used or discarded, classified, selected, released or withheld, shipped, moved to a new location, released into commerce, sold, or offered for sale, withdrawn from commerce or no longer offered for sale, depending on whether the product has expired.

In another aspect, the invention features a method of complying with a regulatory requirement, e.g., a post approval requirement of a regulatory agency, e.g., the FDA. The method includes providing an evaluation of an antibody formulation for a parameter, as described herein. The post approval requirement can include a measure of one more of the above parameters. The method also includes, optionally, determining whether the observed solution parameter meets a preselected criteria or if the parameter is in a preselected range; optionally, memorializing the value or result of the analysis, or communicating with the agency, e.g., by transmitting the value or result to the regulatory agency.

In another aspect, the invention features a method of making a batch of a modified SDAB molecule, e.g., a TNFα SDAB molecule, having a preselected property, e.g., meeting a release specification, label requirement, or compendial requirement, e.g., a property described herein. The method includes providing a test sample containing the modified SDAB molecule; analyzing the test sample according to a method described herein; determining if the test formulation satisfies a preselected criteria, e.g., having a preselected relationship with a reference value, e.g., one or more reference values disclosed herein, and selecting the test sample preparation to make a batch of product.

In another aspect, the invention features multiple batches of a formulation of a modified SDAB molecule, e.g., a TNFα SDAB molecule, wherein one or more parameters (e.g., a value or solution parameter determined by a method described herein), for each batch varies less than a preselected range from a pre-selected desired reference value or criteria, e.g., a range or criteria described herein. In some embodiments, one or more parameters for one or more batches of formulation, is determined and a batch or batches selected as a result of the determination. Some embodiments include comparing the results of the determination to a preselected value or criteria, e.g., a reference standard. Other embodiments include adjusting the dose of the batch to be administered, e.g., based on the result of the determination of the value or parameter.

All publications, patent applications, patents, and other references mentioned herein are incorporated by reference in their entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

Other features and advantages of the invention will be apparent from the detailed description, drawings, and from the claims.

### BRIEF DESCRIPTION OF DRAWINGS

FIG. 1 is the amino acid sequence of SDAB-01 (SEQ ID NO:1). Bold CDRs correspond to the single antigen binding domain building blocks, which each have the amino acid sequence of amino acids 1-115 of SEQ ID NO:1. The flexible linkers are shown in lower case. The engineered C-terminal Cysteine supporting the site specific PEGylation is shown in bold as well.

FIG. 2 is Poly Ethylene Glycol (PEG) used in molecule SDAB-01 (molecular weight 40,000; 2×20 kDa). The PEG activated group is maleimide.

FIG. 3 is a schematic representation of SDAB-01.

FIG. 4A illustrates structures of a linear mPEG-maleimide (Control 2), and two branched mPEG-maleimides ([SEQ ID NO:1]-PEG40 and SDAB-01). FIG. 4B is a scan comparing the sizes of SDAB-01 and [SEQ ID NO:1]-PEG40.

FIG. 5 is a FACS (“fluorescence activated cell sorting”) scan of cell surface staining of SDAB-01 on membrane bound TNFα expressing CHO-TNF-D13 (pW2128) cells. The cells were stained in sequence with SDAB-01, biotinylated anti-PEG, and streptavidin-PE (gray fill) or mock stained followed by streptavidin-PE (white fill).

FIG. 6 represents dose response curves of SDAB-01 in cytotoxicity assays with human or rhesus TNFα in comparison to un-PEGylated SDAB polypeptide Control 3 and a Control 4.

FIG. 7 represents TNFα binding curves to SDAB-01. Various concentrations of (a) human, (b) rhesus macaque, (c) rat and (d) mouse ranging from 0.195 nM to 100 nM, and (e) rabbit TNFα ranging from 0.195 to 400 nM were injected over immobilized SDAB-01. Each data set is representative of at least two independent experiments.

FIG. 8 is a graph depicting the effect of SDAB-01 on total white blood cell infiltrate in experiment 1 in the murine air pouch model.

FIG. 9 is a graph depicting the effect of SDAB-01 on neutrophil infiltration in experiment 1 in the murine air pouch model.

FIG. 10 is a graph depicting the effect of SDAB-01 on total white blood cell infiltrate in experiment 2 in the murine air pouch model.

FIG. 11 is a graph depicting the effect of SDAB-01 on neutrophil infiltration in experiment 2 in the murine air pouch model.

FIG. 12 is a graph depicting the effect of SDAB-01 on total white blood cell infiltrate in experiment 3 in the murine air pouch model.

FIG. 13 is a graph depicting the effect of SDAB-01 on neutrophil infiltration in experiment 3 in the murine air pouch model.

FIG. 14 is a graph showing the body weight by week in animals receiving treatment with SDAB-01 at 10, 3, 1, 0.3, 0.1 mg/kg, control SDAB at 1 mg/kg, Infliximab at 10 and 3 mg/kg, control antibody at 10 mg/kg, or vehicle at 10 ml/kg twice per week.

FIG. 15 a graph showing the mean disease severity scores by week in animals receiving treatment with SDAB-01 at 10, 3, 1, 0.3, 0.1 mg/kg, control SDAB at 1 mg/kg, Infliximab at 10 and 3 mg/kg, control antibody at 10 mg/kg, or vehicle at 10 ml/kg twice per week.

FIG. 16 a graph showing disease severity at 7 weeks post treatment in animals receiving SDAB-01 at 10, 3, 1, 0.3, 0.1 mg/kg, control SDAB at 1 mg/kg, Infliximab at 10 and 3 mg/kg, control antibody at 10 mg/kg, or vehicle at 10 ml/kg twice per week.

FIG. 17 is a graph showing microscopic group mean severity scores at 7 weeks post treatment in animals receiving SDAB-01 at 10, 3, 1, 0.3, 0.1 mg/kg, control SDAB at 1 mg/kg, Infliximab at 10 and 3 mg/kg, control antibody at 10 mg/kg, or vehicle at 10 ml/kg twice per week.

FIG. 18 is a graph showing the comparison of microscopic group mean severity scores and disease severity scores at 7 weeks post treatment in animals receiving SDAB-01 at 10, 3, 1, 0.3, 0.1 mg/kg, control SDAB at 1 mg/kg, Infliximab at 10 and 3 mg/kg, control antibody at 10 mg/kg, or vehicle at 10 ml/kg twice per week.

FIG. 19 is a graph showing body weight by week in animals receiving treatment with SDAB-01 at 10, 3, 1, 0.3, 0.1, 0.03 mg/kg, control SDAB at 1 mg/kg, Infliximab at 10 and 3 mg/kg, control antibody at 10 mg/kg, or vehicle at 10 ml/kg twice per week.

FIG. 20 is a graph showing mean disease severity score by week in animals receiving treatment with SDAB-01 at 10, 3, 1, 0.3, 0.1, 0.03 mg/kg, control SDAB at 1 mg/kg, Infliximab at 10 and 3 mg/kg, control antibody at 10 mg/kg, or vehicle at 10 ml/kg twice per week.

FIG. 21 is a graph showing disease severity scores at 7 weeks post treatment in animals receiving SDAB-01 at 10, 3, 1, 0.3, 0.1, 0.03 mg/kg, control SDAB at 1 mg/kg, Infliximab at 10 and 3 mg/kg, control antibody at 10 mg/kg, or vehicle at 10 ml/kg twice per week.

FIG. 22 is a graph showing the microscopic group mean severity scores post treatment with SDAB-01 at 10, 3, 1, 0.3, 0.1, 0.03 mg/kg, control SDAB at 1 mg/kg, Infliximab at 10 and 3 mg/kg, control antibody at 10 mg/kg, or vehicle at 10 ml/kg twice per week.

FIG. 23 is a graph showing the comparison of microscopic group mean severity scores and disease severity scores at 7 weeks post treatment in animals receiving SDAB-01 at 10, 3, 1, 0.3, 0.1, 0.03 mg/kg, control SDAB at 1 mg/kg, Infliximab at 10 and 3 mg/kg, control antibody at 10 mg/kg, or vehicle at 10 ml/kg twice per week.

FIG. 24 is a graph depicting mean (±SD) serum concentration-time profiles of SDAB-01 in male cynomolgous monkeys after single IV or SC administration of 3 mg/kg.

FIG. 25 is a graph depicting mean (±SD) dose-normalized serum concentrations of PEGylated TNFα SDAB polypeptide after a single IV dose to mice, rats, or cynomolgous monkeys. TNFα SDAB polypeptide 2×20 kDa PEG (filled circles), TNFα SDAB polypeptide 4×10 kDa PEG (open circles), or TNFα SDAB polypeptide 1×40 kDa PEG (filled triangles) were administered a single IV bolus dose to B6CBAF1/J mice (A; 2 mg/kg for the 2×20 kDa PEG conjugate and 3 mg/kg for the other two conjugates); Sprague-Dawley rats (B; 2 mg/kg), or cynomolgous monkeys (C; 3 mg/kg). For the mouse and monkey PK studies (A and C), unlabeled test articles were used, while for the rat PK studies (B), 125I-labeled test articles were used. Non-serial sampling was used for mice (n=3 per time point) and serial sampling was used for rat (n=5-7 per compound) and monkeys (n=3 per compound). Serum concentrations were determined by the specific immunoassays (mice and monkeys; in ng/mL) or gamma-counting (rats; in ng eq./mL). In life duration was 14, 24, and 56-62 days for mice, rats, and monkeys, respectively. Individual animal concentration values below the limit of quantitation (LOQ) were treated as zero for the calculations of the mean and SD. Data show mean (±SD) dose-normalized concentrations at each time point (i.e. for 1 mg/kg dose). Data points with the mean serum concentrations of 0 ng/mL (i.e. below the LOQ for all animals) are not shown on the logarithmic scale.

FIG. 26 is a graph showing the mean tissue and serum exposures (AUC0-168 hr) of 125I-labeled PEGylated TNFα SDAB polypeptides after a single 0.3 mg/kg IV dose to mice. B6CBAF1/J mice were administered a single 0.3 mg/kg IV bolus dose of 125I-labeled TNFα SDAB molecule branched 2×20 kDa PEG (black bars) or TNFα SDAB molecule linear 40 kDa PEG (gray bars). Serum and tissue samples (n=8-12 per time point) were collected over the 7 days (168 hr) and radioactive equivalent (RE) concentrations in tissue and serum were determined by gamma-counting. AUC0-168 hr (area under the concentration-time curve from time 0 to 168 hr). in serum (in μg×eq./mL) and in each tissue (μg×eq./g) were determined by non-compartmental analysis using the sparse sampling method and the 95% confidence interval (95% Cl, the error bars on the graph) was calculated using the standard error of the mean. Star (*) indicates statistically significant difference (p<0.05) in AUC0-168 hr between the two constructs.

FIG. 27 is a graph showing cation exchange high performance liquid chromatography (CEX-HPLC) profiles of PEGylated TNFα SDAB polypeptides. The protein concentration of each material was adjusted to 1.0 mg/mL with formulation buffer and 10 μL was injected onto a Dionex ProPac WCX-10 column. Mobile phase A was 10 mM ammonium formate, pH 4.0. Mobile phase B was 10 mM ammonium formate, 500 mM sodium chloride, pH 4.0. Protein conjugates were eluted at a flow rate of 0.75 mL/min with a linear gradient of sodium chloride (0-40% B in 40 min). Absorbance at 280 nm was monitored.

FIG. 28 is a graph showing size exclusion high performance liquid chromatography with multi-angle light scattering (SEC-MALS) profiles of PEGylated TNFα SDAB polypeptides. TNFα SDAB polypeptide 2×20 kDa PEG (dashed line), TNFα SDAB polypeptide 4×10 kDa PEG (dotted line), or TNFα SDAB polypeptide 1×40 kDa PEG (solid line) were diluted to 2.0 mg/mL and 100 μL of each sample was injected over a Superose 6 mobile phase column held at 30° C. Retention time (lines), total mass (filled circles), PEG mass (open triangles), and protein mass (x) were determined using ASTRA V v5.3.4.14 from Wyatt Technologies.

FIG. 29 is a graph showing the determination of hydrodynamic radii (Rh) and root mean squared radii (RMS or Rg). TNFα SDAB polypeptide 2×20 kDa PEG (dashed line and open squares), TNFα SDAB polypeptide 4×10 kDa PEG (green line and symbols), or TNFα SDAB polypeptide 1×40 kDa PEG (dotted line and open triangles) were diluted to 2.0 mg/mL and 100 μL of each sample was injected over a Superose 6 column mobile phase held at 30° C. Retention times (solid lines and filled circles), Rh (A) and RMS (B) analysis was performed using ASTRA V v5.3.4.14 from Wyatt Technologies.

FIG. 30 is a graph showing the ADCC activity of Control 1, Control 2, Control 3, and a control IgG1 antibody were compared with SDAB-01 using CSFE labeled CHO-TNFD13 (pW2128) cells as targets and human NK cells as effectors. % ADCC activity values are calculated as the % of target cells that are 7AAD+. The values plotted are the % 7AAD+ target cells with the test agents minus the %7AAD+ target cells in the presence of the effectors cell only. This plot is representative of four individual ADCC assays performed that demonstrated no ADCC activity for SDAB-01.

FIG. 31 is a graph showing the CDC activity of Control 1, Control 2, Control 3, and a control IgG1 antibody were compared with SDAB-01 on the CHO-TNF-D13 (pW2128) line in vitro in the presence of baby rabbit complement. Cytotoxicity was assessed by the uptake of 7AAD by dead cells, values plotted are % of 7AAD+ cells with the test and control subtracted from % of 7AAD+ cells in the presence of complement only. The samples were run in duplicates for adalimumab, infliximab and SDAB-01. This plot is representative of three individual assays performed that demonstrated no CDC activity for SDAB-01.

### DETAILED DESCRIPTION

The invention relates to modified single domain antigen binding molecules (also referred to herein as “SDAB molecules.” The modified SDAB molecule can include one or more single antigen binding domains that interact with, e.g., bind to, one or more targets. In one embodiment, one or more of the single antigen binding domains of the modified SDAB molecule bind to tumor necrosis factor-alpha (TNFα). The SDAB molecule can be modified to increase its biological properties in vivo. For example, the SDAB molecule can be modified to improve one or more of: increased half life; reduced immunogenicity; or improve at least one pharmacokinetic/pharmacodynamic (PK/PD) parameter, compared to the unmodified SDAB molecule. In one embodiment, the modified SDAB molecule includes one or more polymer molecules, such as poly(ethyleneglycol) (PEG) or a derivative thereof. The modified SDAB molecules are useful, e.g., for administration to a subject, e.g., a human. Methods of preparing and using the modified SDAB molecules to treat or prevent TNFα-associated disorders are also disclosed.

In order that the present invention may be more readily understood, certain terms are first defined. Additional definitions are set forth throughout the detailed description.

As used herein, the articles “a” and “an” refer to one or to more than one (e.g., to at least one) of the grammatical object of the article.

The term “or” is used herein to mean, and is used interchangeably with, the term “and/or,” unless context clearly indicates otherwise.

The terms “proteins” and “polypeptides” are used interchangeably herein.

“About” and “approximately” shall generally mean an acceptable degree of error for the quantity measured given the nature or precision of the measurements. Exemplary degrees of error are within 20 percent (%), typically within 10%, and more typically within 5% of a given value or range of values.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%. 91%, 92%, 93%, 94%, 95%, 96%, 97%, 98% or 99% identity to a reference sequence.

Also included as polypeptides of the present invention are fragments, derivatives, analogs, or variants of the foregoing polypeptides, and any combination thereof. The terms “fragment,” “variant,” “derivative” and “analog” when referring to proteins of the present invention include any polypeptides which retain at least some of the functional properties of the corresponding native antibody or polypeptide. Fragments of polypeptides of the present invention include proteolytic fragments, as well as deletion fragments, in addition to specific antibody fragments discussed elsewhere herein. Variants of the polypeptides of the present invention include fragments as described above, and also polypeptides with altered amino acid sequences due to amino acid substitutions, deletions, or insertions. Variants may occur naturally or be non-naturally occurring. Non-naturally occurring variants may be produced using art-known mutagenesis techniques. Variant polypeptides may comprise conservative or non-conservative amino acid substitutions, deletions or additions. Derivatives of the fragments of the present invention are polypeptides which have been altered so as to exhibit additional features not found on the native polypeptide. Examples include fusion proteins. Variant polypeptides may also be referred to herein as “polypeptide analogs.” As used herein a “derivative” of a polypeptide refers to a subject polypeptide having one or more residues chemically derivatized by reaction of a functional side group. Also included as “derivatives” are those polypeptides which contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a typical embodiment, the length of a reference sequence aligned for comparison purposes is at least 30%, at least 40%, at least 50% or 60%, or at least 70%, 80%, 90%, or 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch ((1970) J. Mol. Biol. 48:444-453) algorithm which has been incorporated into the GAP program in the GCG software package (available on the worldwide web at gcg dot com), using either a Blossum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available on the worldwide web at gcg dot com), using a NWSgapdna.CMP matrix and a gap weight of 40, 50, 60, 70, or 80 and a length weight of 1, 2, 3, 4, 5, or 6. One typical set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecules featured in the invention. BLAST protein searches can be performed with the XBLAST program, score=50, wordlength=3 to obtain amino acid sequences homologous to a protein (SEQ ID NO:1) molecule featured in the invention. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Various aspects of the invention are described in further detail below.

### Single Domain Antigen Binding (SDAB) Molecules

Single domain antigen binding (SDAB) molecules include molecules whose complementary determining regions are part of a single domain polypeptide. Examples include, but are not limited to, heavy chain variable domains, binding molecules naturally devoid of light chains, Nanobodies™, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or Nanobody™ to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display), as described in more detail below.

The term “antigen-binding” is intended to include the part of a polypeptide, e.g., a single domain molecule described herein, that comprises determinants that form an interface that binds to a target antigen, or an epitope thereof. With respect to proteins (or protein mimetics), the antigen-binding site typically includes one or more loops (of at least four amino acids or amino acid mimics) that form an interface that binds to the target antigen. Typically, the antigen-binding site of the polypeptide, e.g., the single domain antibody molecule, includes at least one or two CDRs, or more typically at least three, four, five, or six CDRs.

The term “immunoglobulin variable domain” is frequently understood in the art as being identical or substantially identical to a VL or a VH domain of human or animal origin. It shall be recognized that immunoglobulin variable domain may have evolved in certain species, e.g., sharks and llama, to differ in amino acid sequence from human or mammalian VL or VH. However, these domains are primarily involved in antigen binding. The term “immunoglobulin variable domain” typically includes at least one or two CDRs, or more typically at least three CDRs.

A “constant immunoglobulin domain” or “constant region” is intended to include an immunoglobulin domain that is identical to or substantially similar to a CL, CH1, CH2, CH3, or CH4, domain of human or animal origin. See e.g. Charles A Hasemann and J. Donald Capra, Immunoglobulins: Structure and Function, in William E. Paul, ed., Fundamental Immunology, Second Edition, 209, 210-218 (1989). The term “Fc region” refers to the Fc portion of the constant immunoglobulin domain that includes immunoglobulin domains CH2 and CH3 or immunoglobulin domains substantially similar to these.

In certain embodiments, the SDAB molecule is a monovalent, or a multispecific molecule (e.g., a bivalent, trivalent, or tetravalent molecule). In other embodiments, the SDAB molecule is a monospecific, bispecific, trispecific or tetraspecific molecule. Whether a molecule is “monospecific” or “multispecific,” e.g., “bispecific,” refers to the number of different epitopes with which a binding polypeptide reacts. Multispecific molecules may be specific for different epitopes of a target polypeptide described herein or may be specific for a target polypeptide as well as for a heterologous epitope, such as a heterologous polypeptide or solid support material.

As used herein the term “valency” refers to the number of potential binding domains, e.g., antigen binding domains, present in an SDAB molecule. Each binding domain specifically binds one epitope. When an SDAB molecule comprises more than one binding domain, each binding domain may specifically bind the same epitope, for an antibody with two binding domains, termed “bivalent monospecific,” or to different epitopes, for an SDAB molecule with two binding domains, termed “bivalent bispecific.” An SDAB molecule may also be bispecific and bivalent for each specificity (termed “bispecific tetravalent molecules”). Bispecific bivalent molecules, and methods of making them, are described, for instance in U.S. Pat. Nos. 5,731,168; 5,807,706; 5,821,333; and U.S. Appl. Publ. Nos. 2003/020734 and 2002/0155537, the disclosures of all of which are incorporated by reference herein. Bispecific tetravalent molecules, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

In certain embodiments, the SDAB molecule is a single chain fusion polypeptide comprising one or more single domain molecules devoid of a complementary variable domain or an immunoglobulin constant, e.g., Fc, region, that binds to one or more target antigens. An exemplary target antigen recognized by the antigen-binding polypeptides includes tumor necrosis factor α (TNFα). In certain embodiment, the antigen-binding single domain molecule is modified by associating, e.g., covalently attaching, the domain to a PEG, e.g., a branched PEG molecule.

### TNFα

Tumor necrosis factor alpha is known in the art to be associated with inflammatory disorders such as rheumatoid arthritis, Crohn's disease, ulcerative colitis and multiple sclerosis. Both TNFα and the receptors (CD120a and CD120b) have been studied in great detail. TNFα in its bioactive form is a trimer. Several strategies to antagonize the action of TNFα using anti-TNFα antibodies have been developed and are currently commercially available, such as Remicade® and Humira®. Antibody molecules against TNFα are known. Numerous examples of TNFα-binding single domain antigen binding molecules are disclosed in WO 2004/041862, WO 2004/041865, WO 2006/122786, the contents of all of which are incorporated by reference herein in their entirety. Additional examples of single domain antigen binding molecules are disclosed in US 2006/286066, US 2008/0260757, WO 06/003388, US05/0271663, US 06/0106203, the contents of all of which are incorporated by reference herein in their entirety. In other embodiments, mono-, bi-, tri- and other multi-specific single domain antibodies against TNFα and a PEG.

As used herein, the terms “TNF,” “TNFα”, and “TNF-alpha” are interchangeable and carry the same meaning.

In specific embodiments, the TNFα-binding SDAB molecule comprises one or more of the SDAB molecules disclosed in Table 11 herein and in WO 2006/122786. For example, the TNFα-binding SDAB molecule can be a monovalent, bivalent, trivalent TNFα-binding SDAB molecule disclosed in WO 2006/122786. Exemplary TNFα-binding SDAB molecules include, but are not limited to, TNF1, TNF2, TNF3, humanized forms thereof (e.g., TNF29, TNF30, TNF31, TNF32, TNF33). Additional examples of monovalent TNFα-binding SDAB molecules are disclosed in Table 8 of WO 2006/122786. Exemplary bivalent TNFα-binding SDAB molecules include, but are not limited to, TNF55 and TNF56, which comprise two TNF30 SDAB molecules linked via a peptide linker to form a single fusion polypeptide (disclosed in WO 2006/122786). Additional examples of bivalent TNFα-binding SDAB molecules are disclosed in Table 19 of WO 2006/122786 as TNF4, TNF5, TNF6, TNF7, TNF8).

In other embodiments, two or more of the single antigen binding domains of the SDAB molecules are fused, with or without a linking group, as a genetic or a polypeptide fusion. The linking group can be any linking group apparent to those of skill in the art. For instance, the linking group can be a biocompatible polymer with a length of 1 to 100 atoms. In one embodiment, the linking group includes or consists of polyglycine, polyserine, polylysine, polyglutamate, polyisoleucine, or polyarginine residues, or a combination thereof. For example, the polyglycine or polyserine linkers can include at least five, seven eight, nine, ten, twelve, fifteen, twenty, thirty, thirty-five and forty glycine and serine residues. Exemplary linkers that can be used include Gly-Ser repeats, for example, (Gly)₄-Ser (SEQ ID NO:8) repeats of at one, two, three, four, five, six, seven or more repeats. In some embodiments, the linker has the following sequences: (Gly)₄-Ser-(Gly)₃-Ser (SEQ ID NO:9) or ((Gly)₄-Ser)ₙ (SEQ ID NO:10), where n is 4, 5, or 6.

In one exemplary embodiment, an antigen-binding polypeptide composed of a single chain polypeptide fusion of two single domain antibody molecules (e.g., two camelid variable regions) that bind to a target antigen, e.g., tumor necrosis factor alpha (TNFα), and a branched PEG molecule was shown to have a dose dependent therapeutic effect on established arthritis in a transgenic mouse model. SDAB-01 is a humanized, bivalent, bi-specific, TNFα-inhibiting fusion protein. The antigen for this protein is tumor necrosis factor-alpha (TNFα).

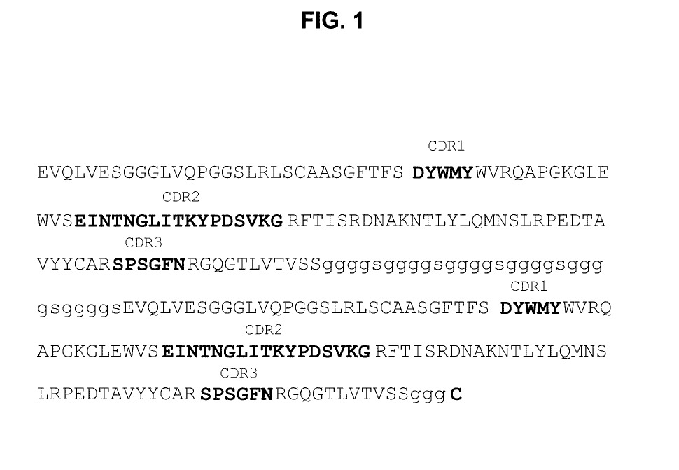

The complete amino acid sequence of the SDAB-01 polypeptide chain predicted from the DNA sequence of the corresponding expression vector is shown in FIG. 1 (residues are numbered starting with the NH₂-terminus as Residue Number 1 of SEQ ID NO:1). The last amino acid residue encoded by the DNA sequence is C²⁶⁴ and constitutes the COOH-terminus of the protein. The predicted molecular mass for disulfide-bonded SDAB-01 (with no posttranslational modifications) is about 27000 Da. The molecular mass observed for the predominant isoform by nanoelectrospray ionization quadrupole time-of-flight mass spectrometry corresponds to 67000 Da confirming the absence of post-translational modifications. The specific biochemical characteristics are as follows: 264 amino acids, 27,365 Da in molecular weight, PI=8.67 and UV=Ec=1.83 at 280 nm.

In FIG. 1, complementarity determining regions (CDR) are in bold. The amino acid linkers connecting these binding domains are in lower case.

### Preparation of SDAB Molecules

The SDAB molecules may be comprised of one or more single domain molecules that are recombinant, CDR-grafted, humanized, camelized, de-immunized, and/or in vitro generated (e.g., selected by phage display). Techniques for generating antibodies and SDAB molecules, and modifying them recombinantly are known in the art and are described in detail below.

Numerous methods known to those skilled in the art are available for obtaining antibodies. For example, monoclonal antibodies may be produced by generation of hybridomas in accordance with known methods. Hybridomas formed in this manner are then screened using standard methods, such as enzyme-linked immunosorbent assay (ELISA) and surface plasmon resonance (BIACORE™) analysis, to identify one or more hybridomas that produce a SDAB molecule that specifically binds with a specified antigen. Any form of the specified antigen may be used as the immunogen, e.g., recombinant antigen, naturally occurring forms, any variants or fragments thereof, as well as antigenic peptide thereof.

One exemplary method of making antibodies and SDAB molecules includes screening protein expression libraries, e.g., phage or ribosome display libraries. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809.

In addition to the use of display libraries, the specified antigen can be used to immunize a non-human animal, e.g., a rodent, e.g., a mouse, hamster, or rat. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, US 2003-0070185, WO 96/34096, published Oct. 31, 1996, and PCT Application No. PCT/US96/05928, filed Apr. 29, 1996.

In another embodiment, an SDAB molecule is obtained from the non-human animal, and then modified, e.g., humanized, deimmunized, chimeric, may be produced using recombinant DNA techniques known in the art. A variety of approaches for making chimeric antibodies and SDAB molecules have been described. See e.g., Morrison et al., Proc. Natl. Acad. Sci. U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., European Patent Publication EP171496; European Patent Publication 0173494, United Kingdom Patent GB 2177096B. Humanized antibodies and SDAB molecules may also be produced, for example, using transgenic mice that express human heavy and light chain genes, but are incapable of expressing the endogenous mouse immunoglobulin heavy and light chain genes. Winter describes an exemplary CDR-grafting method that may be used to prepare the humanized antibodies and SDAB molecule described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody and SDAB molecule to a predetermined antigen.

Humanized antibodies can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an SDAB molecule against a predetermined target, as described above, as well as from other sources. The recombinant DNA encoding the humanized SDAB molecule can then be cloned into an appropriate expression vector.

In certain embodiments, a humanized SDAB molecule is optimized by the introduction of conservative substitutions, consensus sequence substitutions, germline substitutions and/or backmutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982), and may be made according to the teachings of PCT Publication WO92/06193 or EP 0239400).

Techniques for humanizing SDAB molecules are disclosed in WO 06/122786.

An SDAB molecule may also be modified by specific deletion of human T cell epitopes or “deimmunization” by the methods disclosed in WO 98/52976 and WO 00/34317. Briefly, the heavy and light chain variable domains of a SDAB molecule can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable domains, or by single amino acid substitutions. Typically, conservative substitutions are made.

Often, but not exclusively, an amino acid common to a position in human germline antibody sequences may be used. Human germline sequences, e.g., are disclosed in Tomlinson, et al. (1992) J. Mol. Biol. 227:776-798; Cook, G. P. et al. (1995) Immunol. Today Vol. 16 (5): 237-242; Chothia, D. et al. (1992) J. Mol. Biol. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

### Production of SDAB Molecules

The SDAB molecules can be produced by living host cells that have been genetically engineered to produce the protein. Methods of genetically engineering cells to produce proteins are well known in the art. See e.g. Ausabel et al., eds. (1990), Current Protocols in Molecular Biology (Wiley, New York). Such methods include introducing nucleic acids that encode and allow expression of the protein into living host cells. These host cells can be bacterial cells, fungal cells, or animal cells grown in culture. Bacterial host cells include, but are not limited to, Escherichia coli cells. Examples of suitable E. coli strains include: HB101, DH5a, GM2929, JM109, KW251, NM538, NM539, and any E. coli strain that fails to cleave foreign DNA. Fungal host cells that can be used include, but are not limited to, Saccharomyces cerevisiae, Pichia pastoris and Aspergillus cells. A few examples of animal cell lines that can be used are CHO, VERO, BHK, HeLa, Cos, MDCK, 293, 3T3, and WI38. New animal cell lines can be established using methods well know by those skilled in the art (e.g., by transformation, viral infection, and/or selection). Optionally, the protein can be secreted by the host cells into the medium.

In some embodiments, the SDAB molecules can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the periplasm and/or media.

The SDAB molecules can also be produced in eukaryotic cells. In one embodiment, the antibodies (e.g., scFvs) are expressed in a yeast cell such as Pichia (see, e.g., Powers et al. (2001) J Immunol Methods. 251:123-35), Hanseula, or Saccharomyces.

In one embodiment, SDAB molecules are produced in mammalian cells. Typical mammalian host cells for expressing the clone antibodies or antigen-binding fragments thereof include Chinese Hamster Ovary (CHO cells) (including dhfr⁻CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequences encoding the SDAB molecule, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced.

In an exemplary system for recombinant expression of the SDAB molecule, a recombinant expression vector encoding both the antibody heavy chain and the antibody light chain is introduced into dhfr⁻CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells can be cultured to allow for expression of the antibody heavy and light chains and intact antibody is recovered from the culture medium. Standard molecular biology techniques can be used to prepare the recombinant expression vector, transfect the host cells, select for transformants, culture the host cells and recover the antibody molecule from the culture medium. For example, some SDAB molecules can be isolated by affinity chromatography.

SDAB molecules can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody molecule and a signal sequence for secretion. The milk produced by females of such transgenic mammals includes, secreted-therein, the antibody of interest. The antibody molecule can be purified from the milk, or for some applications, used directly.

The binding properties of the SDAB molecules may be measured by any method, e.g., one of the following methods: BIACORE™ analysis, Enzyme Linked Immunosorbent Assay (ELISA), x-ray crystallography, sequence analysis and scanning mutagenesis.

The binding interaction of an SDAB molecule and a target (e.g., TNFα) can be analyzed using surface plasmon resonance (SPR). SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface. The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a molecule to a target. Such data can be used to compare different molecules. Information from SPR can also be used to develop structure-activity relationships (SAR). For example, the kinetic and equilibrium binding parameters of different antibody molecule can be evaluated. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

### Modified SDAB Molecules

The SDAB molecules can have an amino acid sequence that differs at least one amino acid position in one of the framework regions from the amino acid sequence of a naturally occurring domain, e.g., VH domain.

It shall be understood that the amino acid sequences of some SDAB molecules, such as the humanized SDAB molecules, can differ at least one amino acid position in at least one of the framework regions from the amino acid sequences of naturally occurring domain, e.g., a naturally occurring VHI-I domains.

The invention also includes formulations of derivatives of the SDAB molecules. Such derivatives can generally be obtained by modification, and in particular by chemical and/or biological (e.g. enzymatical) modification, of the SDAB molecules and/or of one or more of the amino acid residues that form the SDAB molecules disclosed herein.

Examples of such modifications, as well as examples of amino acid residues within the SDAB molecule sequence that can be modified in such a manner (i.e. either on the protein backbone or on a side chain), methods and techniques that can be used to introduce such modifications and the potential uses and advantages of such modifications will be clear to the skilled person.

For example, such a modification may involve the introduction (e.g. by covalent linking or in any other suitable manner) of one or more functional groups, residues or moieties into or onto the SDAB molecule, and in particular of one or more functional groups, residues or moieties that confer one or more desired properties or functionalities to the SDAB molecules. Examples of such functional groups will be clear to the skilled person.

For example, such modification may comprise the introduction (e.g. by covalent binding or in any other suitable manner) of one or more functional groups that increase the half-life, the solubility and/or the absorption of the SDAB molecule, that reduce the immunogenicity and/or the toxicity of the SDAB molecule, that eliminate or attenuate any undesirable side effects of the SDAB molecule, and/or that confer other advantageous properties to and/or reduce the undesired properties of the SDAB molecule; or any combination of two or more of the foregoing. Examples of such functional groups and of techniques for introducing them will be clear to the skilled person, and can generally comprise all functional groups and techniques mentioned in the general background art cited hereinabove as well as the functional groups and techniques known per se for the modification of pharmaceutical proteins, and in particular for the modification of antibodies or antibody fragments (including ScFvs and -148-single domain antibodies), for which reference is for example made to Remington's Pharmaceutical Sciences, 16th ed., Mack Publishing Co., Easton, Pa. (1980). Such functional groups may for example be linked directly (for example covalently) to a SDAB molecules featured in the invention, or optionally via a suitable linker or spacer, as will again be clear to the skilled person.

### Non-Peptidic Linkers

In the SDAB molecules described herein, the one or more SDAB molecules and/or proteins and the one or more acceptable polymers may be directly linked to each other and/or may be linked to each other via one or more suitable linkers.

Certain terms are defined herein.

The terms “alkoxyl” or “alkoxy” as used herein refers to an alkyl group, as defined below, having an oxygen radical attached thereto. Representative alkoxyl groups include methoxy, ethoxy, propyloxy, tert-butoxy and the like.

The term “alkyl” refers to the radical of saturated aliphatic groups, including straight-chain alkyl groups, and branched-chain alkyl groups. In preferred embodiments, a straight chain or branched chain alkyl has 12 or fewer carbon atoms in its backbone (unless otherwise noted) e.g., from 1-12, 1-8, 1-6, or 1-4. Exemplary alkyl moieties include methyl, ethyl, propyl (e.g., isopropyl), butyl (e.g., isobutyl or t-butyl)

The term “alkylene” refers to a divalent alkyl, e.g., —CH₂—, —CH₂CH2—, and —CH₂CH₂CH₂—.

The term “halo” or “halogen” refers to any radical of fluorine, chlorine, bromine or iodine.

In one embodiment, a linker moiety used to “link” a suitable acceptable polymer to an SDAB molecule described herein is represented by a moiety of formula (I):

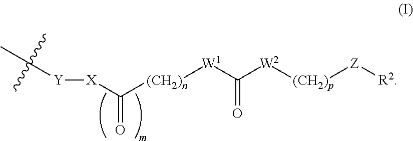

In some embodiments, the linker is represented by the following formula:

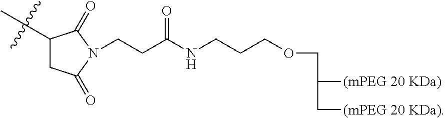

When two or more linkers are used in the SDAB molecules described herein, these linkers may be the same or different. One of ordinary skill in the art would recognize and understand the optimal linkers for use in the SDAB molecule of the invention.

### PEGylation

One widely used technique for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Generally, any suitable form of PEGylation can be used, such as the PEGylation used in the art for antibodies and antibody fragments (including but not limited to (single) domain antibodies and ScFvs); reference is made to for example Chapman, Nat. Biotechnol., 54, 531-545 (2002); by Veronese and Harris, Adv. Drug Deliv. Rev. 54, 453-456 (2003), by Harris and Chess, Nat. Rev. Drug. Discov., 2, (2003) and in WO 04/060965. Various reagents for PEGylation of proteins are also commercially available, for example, from NOF America Corporation (e.g., for PEG formula B). Typically, site-directed PEGylation is used, in particular via a cysteine-residue (see for example Yang et al., Protein Engineering, 16, 10, 761-770 (2003). For example, for this purpose, PEG may be attached to a cysteine residue that naturally occurs in an SDAB molecule, an SDAB molecule may be modified so as to suitably introduce one or more cysteine residues for attachment of PEG. Additionally, a SDAB molecule described herein may be modified so as to suitably introduce one or more cysteine residues for PEGylation, or an amino acid sequence comprising one or more for PEGylation may be fused to the N- and/or C-terminus of a SDAB molecule of the invention, all using techniques of protein engineering.

With regard to PEGylation, it should be noted that generally, the invention also encompasses any SDAB molecule that has been PEGylated at one or more amino acid positions, such as in such a way that said PEGylation either (1) increases the half-life in vivo; (2) reduces immunogenicity; (3) provides one or more further beneficial properties known per se for PEGylation; (4) does not essentially affect the affinity of the SDAB molecule (e.g. does not reduce said affinity by more than 90%, by more than 50%, or by more than 10%, as determined by a suitable assay, such as those described in the Examples below); and/or (4) does not affect any of the other desired properties of the SDAB molecule. Suitable PEG-groups and methods for attaching them, either specifically or non-specifically, will be clear to the skilled person.

A PEG used in the SDAB molecules and proteins described herein can have a molecular weight of 1 KDa or greater, such as 10 KDa and less than 200 KDa, such as 90 KDa. In some embodiments, a PEG used in the SDAB molecules and proteins described herein may have a molecular weight in the range of 1 KDa to 100 KDa. Typically, for the SDAB molecule, a PEG is used with a molecular weight of more than 5000, such as more than 10,000 and less than 200,000, such as less than 100,000; for example in the range of 20,000-80,000. In some embodiments, a PEG used in the SDAB molecules and proteins described herein may have a molecular weight in the range of 10 KDa to 50 KDa. In some embodiments, a PEG used in the SDAB molecules and proteins described herein may have a molecular weight in the range of 15 KDa to 45 KDa. In some embodiments, a PEG used in the SDAB molecules and proteins described herein may have a molecular weight of 20 KDa. In some embodiments, a PEG used in the SDAB molecules and proteins described herein may have a molecular weight of 40 KDa. In some embodiments, a PEG used in the SDAB molecules and proteins described herein may have a molecular weight of 10 KDa.

In some embodiments, each PEG molecule is independently a PEG monomer, polymer or a derivative thereof. In some embodiments, each PEG is a methoxy PEG derivative (mPEG) monomer, polymer or a derivative thereof. In some embodiments, each PEG molecule independently has a molecular weight between 1 KDa and 100 KDa. In some embodiments, each PEG molecule independently has a molecular weight between 10 KDa and 50 KDa. In some embodiments, each PEG molecule independently has a molecular weight of 40 KDa. In some embodiments, each PEG molecule independently has a molecular weight of between 15 KDa and 35 KDa. In some embodiments, each PEG molecule independently has a molecular weight of 30 KDa. In some embodiments, each PEG molecule independently has a molecular weight of 20 KDa. In some embodiments, each PEG molecule independently has a molecular weight of 17.5 KDa. In some embodiments, each PEG molecule independently has a molecular weight of 12.5 KDa. In some embodiments, each PEG molecule independently has a molecular weight of 10 KDa. In some embodiments, each PEG molecule has a molecular weight of 7.5 KDa. In some embodiments, each PEG molecule independently has a molecular weight of 5 KDa.

Another, usually less typical modification comprises N-linked or O-linked glycosylation, usually as part of co-translational and/or post-translational modification, depending on the host cell used for expressing the SDAB molecule.

In some embodiments, the PEG molecule is branched. In some embodiments, the PEG molecule is selected from a moiety of formulas (a)-(h);

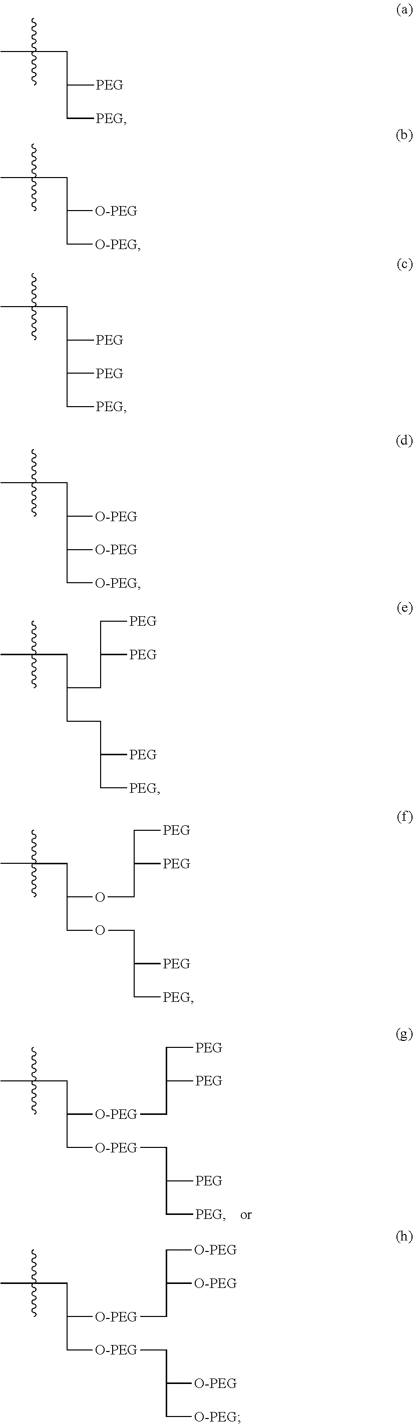

wherein each PEG molecule is independently a PEG monomer, polymer or a derivative thereof. In some embodiments, each PEG molecule is an mPEG monomer, polymer or a derivative thereof. In some embodiments, the modified SDAB molecule includes a linker of formula (I) linked to a PEG molecule and has a structure selected from:

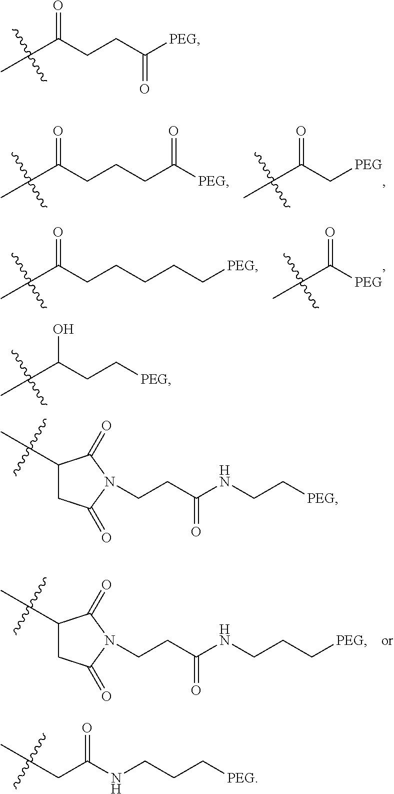

wherein each PEG molecule is independently a PEG monomer, polymer or a derivative thereof. In some embodiments, each PEG molecule is an mPEG monomer, polymer or a derivative thereof.

In some embodiments, the modified SDAB molecule includes a linker of formula (I) linked to a PEG molecule and has a structure selected from:

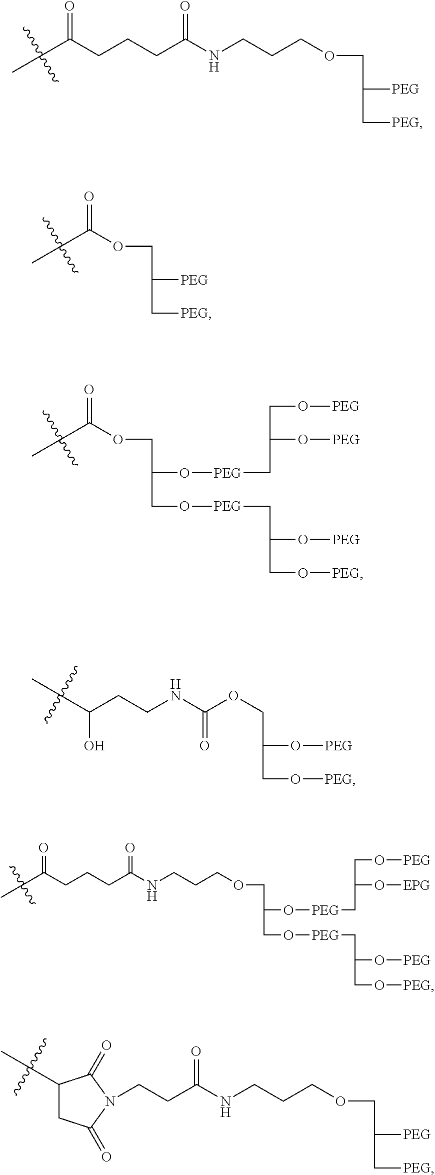

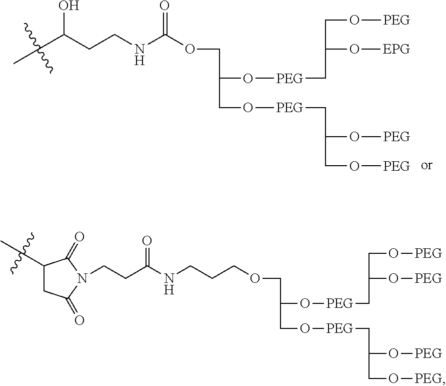

wherein each PEG molecule is independently a PEG monomer, polymer or a derivative thereof. In some embodiments, each PEG molecule is an mPEG monomer, polymer or a derivative thereof.

In some embodiments, the linker of formula (I) is linked to a PEG molecule represented by the following formula:

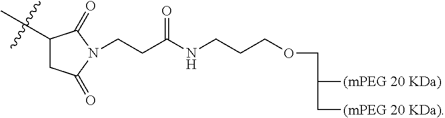

In some embodiments, the linker of formula (I) is linked to a PEG molecule represented by the following formula:

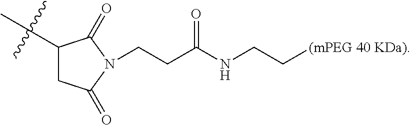

In some embodiments, the linker of formula (I) is linked to a PEG molecule represented by the following formula:

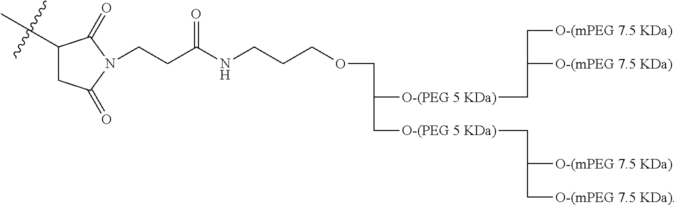

The linker-PEG molecule can be associated with (e.g., coupled to) the SDAB molecule, thereby forming a modified SDAB molecule. The single domain molecules of the SDAB molecule can be arranged in the following order from N- to C-terminus: TNFα-binding single domain molecule—TNFα-binding single domain molecule—PEG molecule (e.g., branched PEG molecule). In one embodiment, the modified SDAB molecule is represented by the following formula:

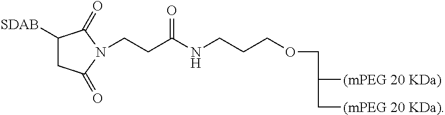

In one embodiment, the modified SDAB molecule is represented by the following formula:

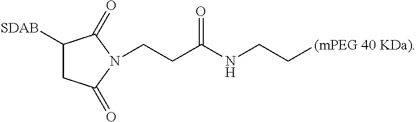

In one embodiment, the modified SDAB molecule is represented by the following formula:

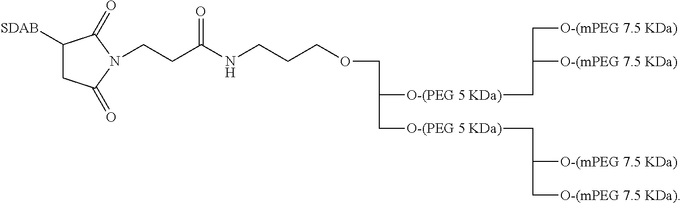

One exemplary embodiment of the modified SDAB molecule is represented by the following formula:

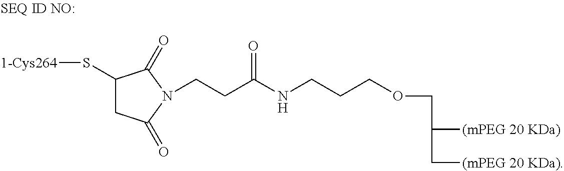

The reactive group of the SDAB molecule is generally attached via a nucleophilic moiety attached to the SDAB molecule. In some embodiments, the nucleophilic moiety is a sulfur (e.g., a sulfur from a cysteine residue). In other embodiments, the nucleophilic moiety is a nitrogen (e.g., from a terminal alpha-amino group or a nitrogen containing amino acid side chain (e.g., an ε-amino group from a lysine chain). In other embodiments, the nucleophilic moiety is a C-terminal group. The reactive group of the SDAB molecule is generally attached via an electrophilic moiety attached to the linker. In some embodiments, the electrophilic moiety is a carbonyl group (e.g., an activated ester or an aldehyde). In some embodiments, the electrophilic moiety is a maleimide group.

### Administration and Method of Treatment

SDAB molecules can be administered to a subject (e.g., a human subject) alone or combination with a second agent, e.g., a second therapeutically or pharmacologically active agent, to treat or prevent (e.g., reduce or ameliorate one or more symptoms associated with) a TNFα associated disorder, e.g., inflammatory or autoimmune disorders. The term “treating” refers to administering a therapy in an amount, manner, and/or mode effective to improve a condition, symptom, or parameter associated with a disorder or to prevent progression of a disorder, to either a statistically significant degree or to a degree detectable to one skilled in the art. In the case of therapeutic use, the treatment may improve, cure, maintain, or decrease duration of, the disorder or condition in the subject. In therapeutic uses, the subject may have a partial or full manifestation of the symptoms. In a typical case, treatment improves the disorder or condition of the subject to an extent detectable by a physician, or prevents worsening of the disorder or condition. An effective amount, manner, or mode can vary depending on the subject and may be tailored to the subject.

As used herein, the terms “subject” and “patient” are used interchangeably. As used herein, the terms “subject” and “subjects” refer to an animal, e.g., a mammal including a non-primate (e.g., a cow, pig, horse, donkey, goat, camel, cat, dog, guinea pig, rat, mouse, sheep) and a primate (e.g., a monkey, such as a cynomolgous monkey, gorilla, chimpanzee and a human).

Non-limiting examples of immune disorders that can be treated include, but are not limited to, autoimmune disorders, e.g., arthritis (including rheumatoid arthritis, juvenile rheumatoid arthritis, osteoarthritis, psoriatic arthritis, lupus-associated arthritis or ankylosing spondylitis), scleroderma, systemic lupus erythematosis, Sjogren's syndrome, vasculitis, multiple sclerosis, autoimmune thyroiditis, dermatitis (including atopic dermatitis and eczematous dermatitis), myasthenia gravis, inflammatory bowel disease (IBD), Crohn's disease, colitis, diabetes mellitus (type I); inflammatory conditions of, e.g., the skin (e.g., psoriasis); acute inflammatory conditions (e.g., endotoxemia, sepsis and septicemia, toxic shock syndrome and infectious disease); transplant rejection and allergy. In one embodiment, the TNFα associated disorder is, an arthritic disorder, e.g., a disorder chosen from one or more of rheumatoid arthritis, juvenile rheumatoid arthritis (RA) (e.g., moderate to severe rheumatoid arthritis), osteoarthritis, psoriatic arthritis, or ankylosing spondylitis, polyarticular juvenile idiopathic arthritis (JIA); or psoriasis, ulcerative colitis, Crohn's disease, inflammatory bowel disease, and/or multiple sclerosis.

In certain embodiments, the SDAB molecules (or formulations) are administered in combination with a second therapeutic agent. For example, for TNFα SDAB molecules, the second agent may be an anti-TNFα antibody or TNFα binding fragment thereof, wherein the second TNFα antibody has a different epitope specificity than the TNFα-binding SDAB molecule of the formulation. Other non-limiting examples of agents that can be co-formulated with TNFα-binding SDAB include, for example, a cytokine inhibitor, a growth factor inhibitor, an immunosuppressant, an anti-inflammatory agent, a metabolic inhibitor, an enzyme inhibitor, a cytotoxic agent, and a cytostatic agent. In one embodiment, the additional agent is a standard treatment for arthritis, including, but not limited to, non-steroidal anti-inflammatory agents (NSAIDs); corticosteroids, including prednisolone, prednisone, cortisone, and triamcinolone; and disease modifying anti-rheumatic drugs (DMARDs), such as methotrexate, hydroxychloroquine (Plaquenil) and sulfasalazine, leflunomide (Arava®), tumor necrosis factor inhibitors, including etanercept (Enbrel®), infliximab (Remicade®) (with or without methotrexate), and adalimumab (Humira®), anti-CD20 antibody (e.g., Rituxan®), soluble interleukin-1 receptor, such as anakinra (Kineret), gold, minocycline (Minocin®), penicillamine, and cytotoxic agents, including azathioprine, cyclophosphamide, and cyclosporine. Such combination therapies may advantageously utilize lower dosages of the administered therapeutic agents, thus avoiding possible toxicities or complications associated with the various monotherapies.

The SDAB molecule can be administered in the form of a liquid solution (e.g., injectable and infusible solutions). Such compositions can be administered by a parenteral mode (e.g., subcutaneous, intraperitoneal, or intramuscular injection), or by inhalation. The phrases “parenteral administration” and “administered parenterally” as used herein mean modes of administration other than enteral and topical administration, usually by injection, and include, subcutaneous or intramuscular administration, as well as intravenous, intracapsular, intraorbital, intracardiac, intradermal, intraperitoneal, transtracheal, subcuticular, subcapsular, subarachnoid, intraspinal, epidural, and intrasternal injection and infusion. In one embodiment, the formulations described herein are administered subcutaneously.

Pharmaceutical compositions or formulations are sterile and stable under the conditions of manufacture and storage. A pharmaceutical composition can also be tested to insure it meets regulatory and industry standards for administration.

A pharmaceutical composition can be formulated as a solution, microemulsion, dispersion, liposome, or other ordered structure suitable to high protein concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

### Compositions/Formulations

A formulation of an SDAB molecule includes a SDAB molecule, a compound that can serve as a cryoprotectant, and a buffer. The pH of the formulation is generally pH 5.5-7.0. In some embodiments, a formulation is stored as a liquid. In other embodiments, a formulation is prepared as a liquid and then is dried, e.g., by lyophilization or spray-drying, prior to storage. A dried formulation can be used as a dry compound, e.g., as an aerosol or powder, or reconstituted to its original or another concentration, e.g., using water, a buffer, or other appropriate liquid.

The SDAB molecule purification process is designed to permit transfer of an SDAB molecule into a formulation suitable for long-term storage as a frozen liquid and subsequently for freeze-drying (e.g., using a histidine/sucrose formulation). The formulation is lyophilized with the protein at a specific concentration. The lyophilized formulation can then be reconstituted as needed with a suitable diluent (e.g., water) to resolubilize the original formulation components to a desired concentration, generally the same or higher concentration compared to the concentration prior to lyophilization.

The lyophilized formulation may be reconstituted to produce a formulation that has a concentration that differs from the original concentration (i.e., before lyophilization), depending upon the amount of water or diluent added to the lyophilate relative to the volume of liquid that was originally freeze-dried. Suitable formulations can be identified by assaying one or more parameters of antibody integrity.

### Articles of Manufacture

The present application also provides an article of manufacture that includes a formulation as described herein and provides instructions for use of the formulation.

Formulations to be used for administration to a subject, e.g., as a pharmaceutical, must be sterile. This is accomplished using methods known in the art, e.g., by filtration through sterile filtration membranes, prior to, or following, formulation of a liquid or lyophilization and reconstitution. Alternatively, when it will not damage structure, components of the formulation can be sterilized by autoclaving and then combined with filter or radiation sterilized components to produce the formulation.

The pharmaceutical formulation can be administered with a transcutaneous delivery device, such as a syringe, including a hypodermic or multichamber syringe. In one embodiment, the device is a prefilled syringe with attached or integral needle. In other embodiments, the device is a prefilled syringe not having a needle attached. The needle can be packaged with the prefilled syringe. In one embodiment, the device is an auto-injection device, e.g., an auto-injector syringe. In another embodiment the injection device is a pen-injector. In yet another embodiment, the syringe is a staked needle syringe, luer lock syringe, or luer slip syringe. Other suitable delivery devices include stents, catheters, microneedles, and implantable controlled release devices. The composition can be administered intravenously with standard IV equipment, including, e.g., IV tubings, with or without in-line filters.

In certain embodiments, a syringe is suitable for use with an autoinjector device. For example, the autoinjector device can include a single vial system, such as a pen-injector device for delivery of a solution. Such devices are commercially available from manufacturers such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, DosePro®, Medi-Ject®, e.g., as made or developed by Becton Dickensen (Franklin Lakes, N.J.), Ypsomed (Burgdorf, Switzerland, on the worldwide web at ypsomed dot com; Bioject, Portland, Oreg.; National Medical Products, Weston Medical (Peterborough, UK), Medi-Ject Corp (Minneapolis, Minn.), and Zogenix, Inc, Emeryville, Calif. Recognized devices comprising a dual vial system include those pen-injector systems for reconstituting a lyophilized drug in a cartridge for delivery of the reconstituted solution such as the HumatroPen®.

The article of manufacture can include a container suitable for containing the formulation. A suitable container can be, without limitation, a device, bottle, vial, syringe, test tube, nebulizer (e.g., ultrasonic or vibrating mesh nebulizers), i.v. solution bag, or inhaler (e.g., a metered dose inhaler (MDI) or dry powder inhaler (DPI)). The container can be formed of any suitable material such as glass, metal, or a plastic such as polycarbonate, polystyrene, or polypropylene.

In general, the container is of a material that does not adsorb significant amounts of protein from the formulation and is not reactive with components of the formulation.

The articles of manufacture described herein can further include a packaging material. The packaging material provides, in addition to the information for use or administration, e.g., information required by a regulatory agency regarding conditions under which the product can be used. For example, the packaging material can provide instructions to the patient on how to inject a pre-filled syringe containing the formulations described herein, or how to reconstitute the lyophilized formulation in an aqueous diluent to form a solution within a specified period, e.g., over a period of 2-24 hours or greater. The presently claimed formulations are useful for human pharmaceutical product use.

In certain embodiments, the formulations can be administered as nebulizers. Examples of nebulizers include, in non-limiting examples, jet nebulizers, ultrasonic nebulizers, and vibrating mesh nebulizers. These classes use different methods to create an aerosol from a liquid. In general, any aerosol-generating device that can maintain the integrity of the protein in these formulations is suitable for delivery of formulations as described herein.

In other embodiments, the pharmaceutical compositions can be administered with medical devices. For example, pharmaceutical compositions can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. The therapeutic composition can also be in the form of a biodegradable or nonbiodegradable sustained release formulation for subcutaneous or intramuscular administration. See, e.g., U.S. Pat. Nos. 3,773,919 and 4,767,628 and PCT Application No. WO 94/15587. Continuous administration can also be achieved using an implantable or external pump. The administration can also be conducted intermittently, e.g., single daily injection, or continuously at a low dose, e.g., sustained release formulation. The delivery device can be modified to be optimally suited for administration of the SDAB molecule. For example, a syringe can be siliconized to an extent that is optimal for storage and delivery of the SDAB molecule. Of course, many other such implants, delivery systems, and modules are also known.

The invention also features a device for administering a first and second agent. The device can include, e.g., one or more housings for storing pharmaceutical preparations, and can be configured to deliver unit doses of the first and second agent. The first and second agents can be stored in the same or separate compartments. For example, the device can combine the agents prior to administration. It is also possible to use different devices to administer the first and second agent.

The Examples that follow are set forth to aid in the understanding of the inventions but are not intended to, and should not be construed to, limit its scope in any way.

### EXAMPLES

### Example 1

### Generation of anti-TNFα Building Blocks and Engineering of SDAB-01

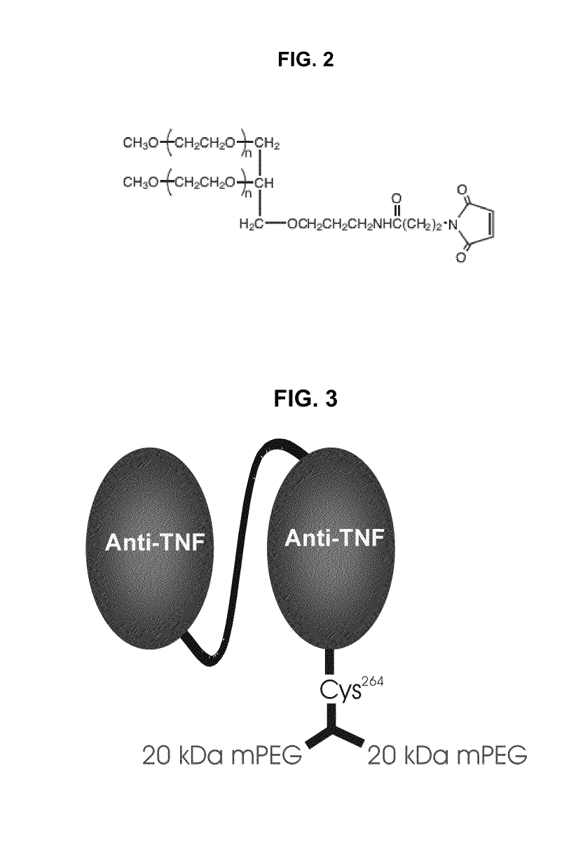

The SDAB-01 bivalent humanized SDAB polypeptide was constructed via genetic fusion of two identical TNFα antigen binding domains (amino acids 1-115 of SEQ ID NO: 1) through a flexible 30 amino acid linker composed of 6 repeats of 4 glycines and 1 serine. To prepare for site specific PEGylation, a free cysteine was engineered at the C-terminus following a three glycine amino acid linker (FIG. 1). The protein was produced in a CHO mammalian expression system and purified by protein A affinity capture. The C-terminal cysteine was then reduced by dithiothreitol treatment and reacted to the maleimide function of an activated 2×20 kDa branched PEG (FIG. 2). The final product was further purified from free PEG and a small proportion of unPEGylated protein and characterized.

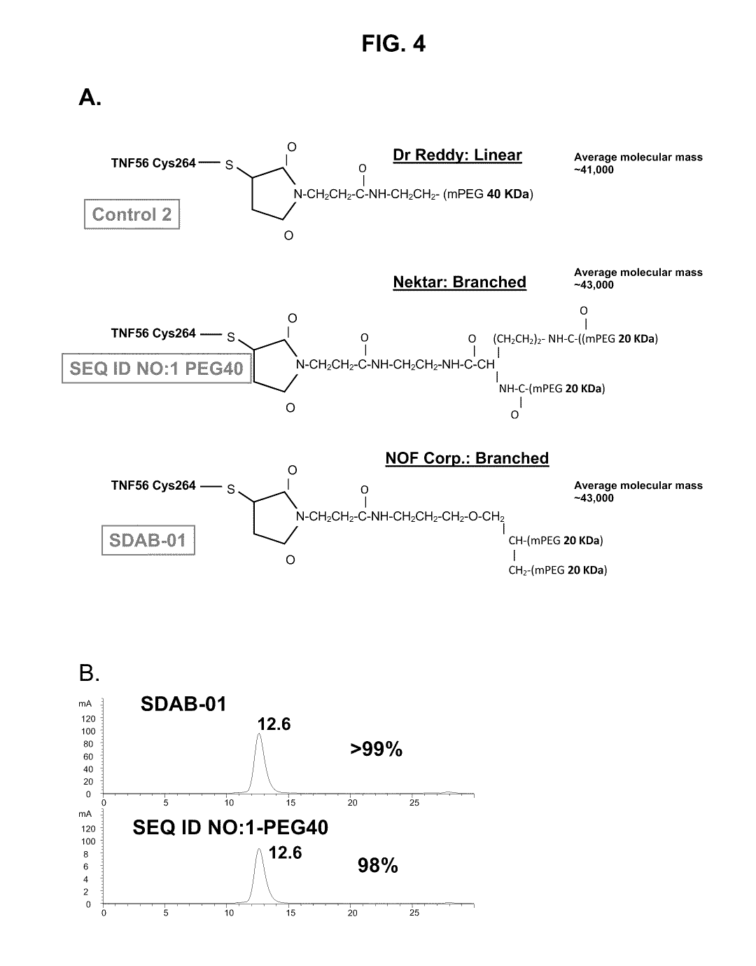

SDAB-01 thereby comprises a genetic fusion of two identical humanized anti-TNFα specific SDAB molecules having the amino acid sequence of amino acids 1-115 of SEQ ID NO:1 separated by a 30 amino acid flexible linker and a C-terminal cysteine site specifically PEGylated (2×20 PEG) with maleimide derivatized 40 kDa (2×20 kDa) branched polyethylene glycol (FIG. 3). FIG. 4A illustrates a linear and two branched mPEG-maleimides, including SDAB-01. FIG. 4B is a scan comparing the sizes of SDAB-01 and [SEQ ID NO:1]-PEG40.

Analytical analyses indicated that the PEGylation efficiency between the linear 40K mPEG-maleimide and the branched 40K mPEG-maleimide SDAB was comparable. Anti-TNFα SDAB molecule PEGylated with either linear or branched 40K mPEG-maleimide showed comparable bioactivity. The apparent charge and shape appears to be most comparable between the two branched 40K mPEG-maleimide materials (branched PEG formula A and branched PEG formula B).

The construction of SDAB-01 as a bivalent format of two identical TNFα antigen binding domains (amino acids 1-115 of SEQ ID NO:1) through a length optimized flexible linker improved its potency by about fifty fold in the cell based TNFα neutralization assay as compared to its monovalent format. The site specific PEGylation of the engineered C-terminal cysteine gave the drug candidate the desired pharmacokinetic profile with extended in-vivo half-life without affecting its potency.

### Example 2

### Binding Characterization of SDAB-01 to Membrane Bound TNFα by Flow Cytometry

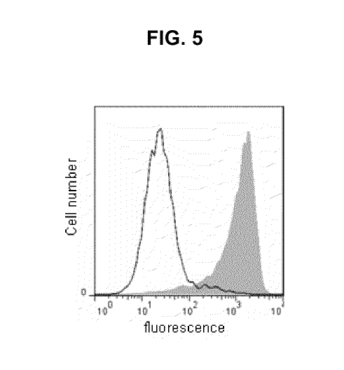

SDAB-01 has been demonstrated by flow cytometry to bind to a recombinant Chinese hamster ovary (CHO) cell line expressing human TNFα on its cell surface. A 13 amino acid deletion was introduced into the human TNFα coding region by site directed mutagenesis to reduce the proteolytic cleavage resulting in the release of TNFα into the media. A stable CHO line was generated using this construct. Expression of TNFα on the cell surface was demonstrated by flow cytometry using a specific anti-human TNFα antibody. SDAB-01 was used to stain the cell line pW2128 CHO-TNF-D13 followed by a secondary stain with a biotinylated anti-PEG antibody and then detected with a tertiary stain with streptavidin-PE demonstrating effecting cell surface binding (FIG. 5)

### Example 3

### Affinity of SDAB-01 on Human or Rhesus TNF

Detailed characterization of anti-TNFα SDAB-01 binding to human and rhesus TNFα was carried out using surface plasmon resonance on a Biacore instrument. Biotinylated SDAB-01 was captured onto a streptavidin sensor chip surface and various concentrations of human or rhesus TNFα were tested in this experiment. TNFα protein was injected and allowed to associate for 1.5 minutes at 100 μL/min and to dissociate for 20 minutes. Rate constants and Kd were determined by global fit using a 1:1 binding model in Biaevaluation software v4.1. Data shown for the rate constants are the mean and standard deviation from at least 2 independent experiments. The Kd was calculated from the mean of the on and off rates. Affinity of SDAB-01 on human or rhesus TNFα is shown in Table 1.

TABLE 1
Affinity of SDAB-01 on human or rhesus TNFα determined by Biacore

| | human TNFα | | | rhesus macaque TNFα | | |
|---|---|---|---|---|---|---|
| | Kon × 10⁶ | Koff × 10⁻⁵ | | Kon × 10⁶ | Koff × 10⁻⁵ | |
| | (1/Ms) | (1/s) | Kd (pM) | (1/Ms) | (1/s) | Kd (pM) |
| SDAB-01 | 7.76 ± 1.62 | 14.7 ± 0.45 | 18.9 | 4.19 ± 0.413 | 14.1 ± 2.53 | 33.7 |
| CONTROL 2 | 7.36 ± 1.31 | 14.8 ± 0.961 | 20.7 | 4.21 ± 0.056 | 13.5 ± 2.53 | 33.7 |

### Example 4

### Characterization of SDAB-01 in Cell-Based Cytotoxicity Assays

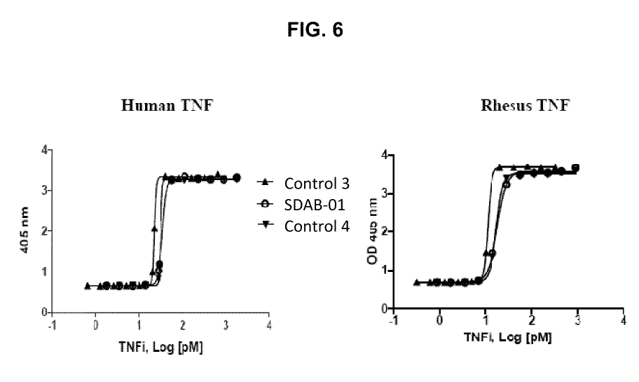

Evaluation of Bioactivity of SDAB-01 in L929 Cell-Based Cytotoxicity Assays Using Human or Rhesus TNFα in Comparison with a Control 4 SDAB molecule and Un-PEGylated SDAB molecule Control 3. The ability of SDAB-01 to neutralize the cytotoxicity of TNFα (0.5 ng/ml) was evaluated in a cell-based dose-response assay. SDAB-01 and Control 3, which is the un-PEGylated TNFα SDAB molecule, were assayed in the same experiment. The dose response curves are shown in FIG. 6 and the EC50 results are summarized in Table 2.

TABLE 2
Bioactivities of SDAB Control 4, SDAB-01
and un-PEGylated Control 3

| | Control 4 | SDAB-01 | Control 3 |
|---|---|---|---|
| | EC50, pM | EC50, pM | EC50, pM |
| EC50, pM | (SD) | (SD) | (SD) |
| Human TNFα | 22.56 | 35.01 | 31.75 |
| | (0.08) | (0.12) | (0.21) |
| Rhesus TNFα | 11.38 | 18.09 | 17.35 |
| | (0.15) | (0.07) | (0.18) |

These results show that SDAB-01 is able to neutralize both human and rhesus TNFα in L929 cell-based assay. The results also indicate that PEGylation does not have a significant effect on SDAB-01's neutralization activity.

### Example 5

### Comparison of TNFα SDAB-01 Binding Kinetics to Different Species of TNFα

The objective of this study was to investigate the binding rates and equilibrium dissociation constant between SDAB-01 and different species of TNFα including human, rhesus macaque, rat, mouse, and rabbit to understand how the binding affinity compares between these different species that may be used for efficacy, pharmacokinetic and toxicology models. A Biacore instrument was used to measure kinetic binding in real time by surface plasmon resonance. Rate constants were directly measured and the equilibrium dissociation constants were derived from the binding rates using Biacore evaluation software v4.1.

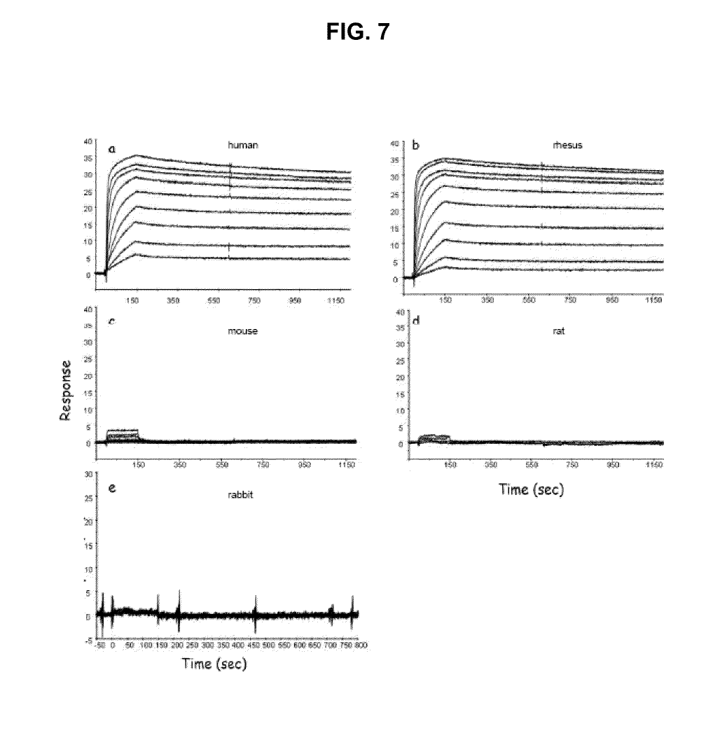

For evaluation of TNFα binding, SDAB-01 was immobilized on a sensor chip surface at densities between 60 to 75 RU. Human and rhesus TNFα bound similarly to SDAB-01 with fast on rates and very slow off rates (FIG. 7a,b). Binding to SDAB-01 was dependent on the concentration of human and rhesus TNFα and reached saturation. At the highest concentrations, the binding reached equilibrium. In contrast to the high affinity binding of SDAB-01 to human and rhesus TNFα, there was negligible binding of rat and mouse TNFα to SDAB-01 (FIG. 7c,d). A very low signal for binding was observed for rat and mouse TNFα binding at the highest concentration tested, 100 nM, and reached less than 5 R^{U} binding response (FIG. 7). The apparent fast off rates and lack of saturation at the highest concentrations tested, up to 100 nM are an indication of weak binding. An equilibrium dissociation constant was not possible to calculate for rat or mouse TNFα, due to the lack of saturation and binding rates that were too fast to measure. This suggests that although there is some negligible binding, rat and mouse TNFα, bind SDAB-01 extremely weakly. No binding of rabbit TNFα was observed to SDAB-01, even at 400 nM of rabbit TNFα, the highest concentration tested (FIG. 7). These data indicate that SDAB-01 will bind to rhesus TNFα similarly to human, but will not engage the TNFα ligand in mouse, rat, or rabbits.

The association and dissociation rate constants between human and rhesus TNFα binding to SDAB-01 were calculated from the binding shown in FIG. 7 using a 1:1 binding model (Table 3). Human and rhesus TNFα had very similar on and off rates, resulting in nearly identical Kd values of 19.5+4.17 and 34.1+7.23 μM, respectively.

TABLE 3
Binding affinities of SDAB-01 to Human and Rhesus TNFα

| | ka (M−1s−1) | kd (s−1) | Kd (pM) |
|---|---|---|---|
| Human | 7.8 +/− 1.6E+06 | 14.7 +/− 0.45E−05 | 19.5 +/− 4.17 |
| TNFα | | | |
| Rhesus | 4.19 +/− 0.41E+06 | 14.1 +/− 2.53E−05 | 34.1 +/− 7.23 |
| macaque | | | |
| TNFα | | | |

### Example 6

### Lack of Complement Dependent Cytotoxicity and Antibody Dependent Cellular Cytotoxicity for SDAB-01

SDAB-01 exhibits high neutralization potency for human and monkey TNFα. Both CDC and ADCC are Fc mediated effector functions. CDC can occur when C1q the first protein in the alternative complement cascade binds to the CH2 domain of the Fc region on two or more IgG molecules. This triggers downstream complement pathway components that ultimately result in the formation of a membrane attack complex on the surface of the cell leading to its lysis. ADCC can trigger killing of the target cells through interaction between the Fc regions of the anti-TNFα antibodies bound to TNFα on the cell surface and FcγRs expressed on immune effectors cells such as NK cells, monocytes, macrophages, and neutrophils. The aim of this study was to test for CDC and ADCC activity by SDAB-01, and to compare it to anti-TNFα antibody control 1 and anti-TNFα antibody control 2, anti-TNFα antibody control 3. Antibody controls 1 and 2 have a human IgG1 Fc and can therefore have effector functions. Antibody control 3 and SDAB-01, lack an Fc region.

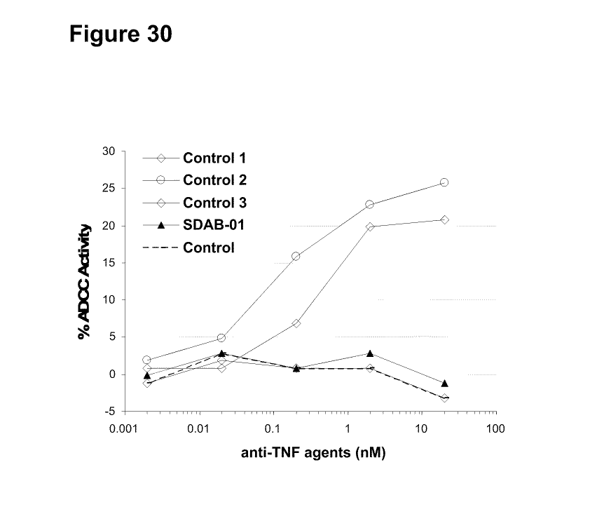

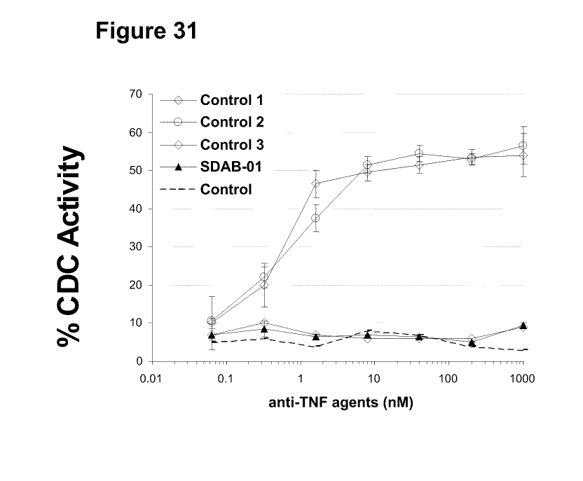

The analysis demonstrated that SDAB-01, and antibody control 3 did not have any CDC and ADCC activity, as compared with antibody controls 1 and 2 (FIGS. 30 & 31). The Fc region of an antibody is required for a molecule to mediate CDC and ADCC activity, and Antibody control 3 and SDAB-01 lack a Fc region. SDAB-01 therefore can potentially bind and neutralize TNFα on the cell surface without causing any effector function activity that could be cytotoxic.

### Example 7

### Effect of SDAB-01 on Neutrophil Infiltration

The purpose of these in vivo studies was to evaluate the ability of different doses of SDAB-01 to decrease cellular infiltration induced by recombinant human TNFα in the murine air pouch model.

Tessier et al. (Jour of Immunol. 159:3595-3602, 1997) have previously shown that injection of TNFα into a mouse air pouch induces an accumulation of leukocytes into the pouch. SDAB-01 was designed to bind and neutralize the effects of TNFα. To test whether SDAB-01 would have an effect on cellular accumulation in an in vivo model, SDAB-01 was administered to mice prior to injection of TNFα into the air pouch. Cells were harvested from the pouch and differentially counted 6 hours after the TNFα administration.

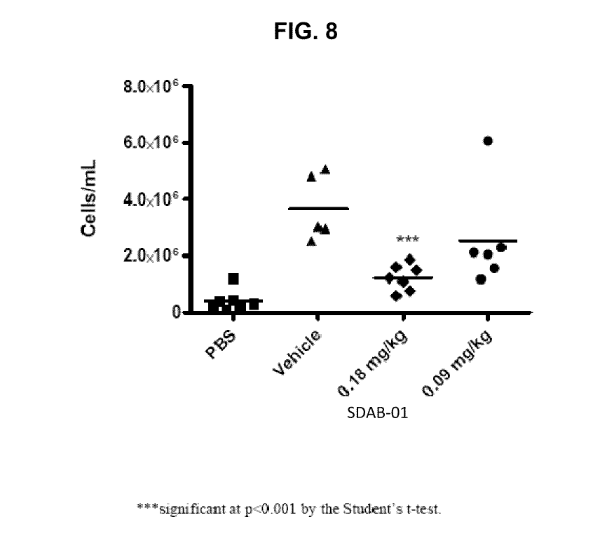

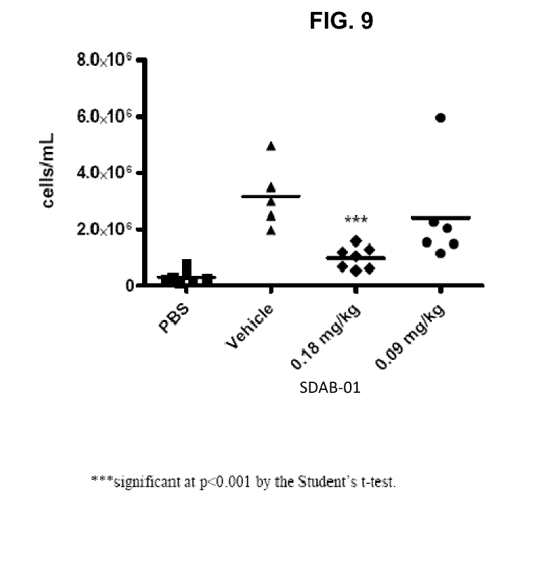

The pouch fluid was collected at the end of each experiment (6 hours after administration of TNFα) and cell counts were determined on the Cell Dyne. The results of experiment 1 are shown in FIG. 8 and FIG. 9.

SDAB-01 dosed at 0.18 mg/kg significantly decreased cellular infiltration into the air pouch induced by 10 ng of recombinant human TNFα. Neutrophil accumulation was also significantly inhibited with 0.18 mg/kg SDAB-01. Lymphocyte and monocyte infiltration were a minor component of the cellular infiltration at the 6 hour time point and this was unaffected by SDAB-01 in this study.

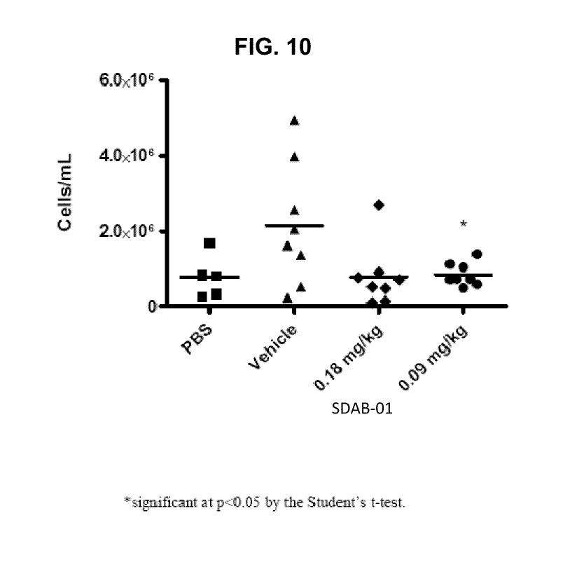

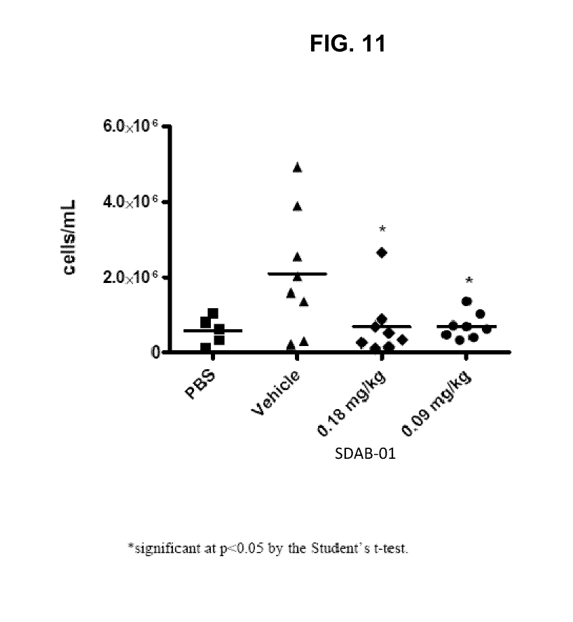

Experiment 2 was carried out using the same protocol as experiment 1 and the results are shown in FIG. 10 and FIG. 11. The results were consistent with those observed in Experiment 1 except that in this experiment, neutrophil infiltration was significantly inhibited by both the 0.18 mg/kg and the 0.09 mg/kg doses of SDAB-01. Total cellular infiltration was significantly decreased by SDAB-01 0.09 mg/kg only, whereas no significant decrease was observed with monocyte or lymphocyte infiltration.

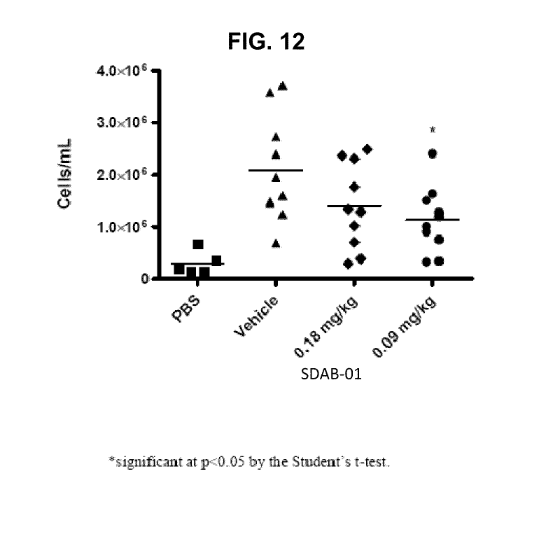

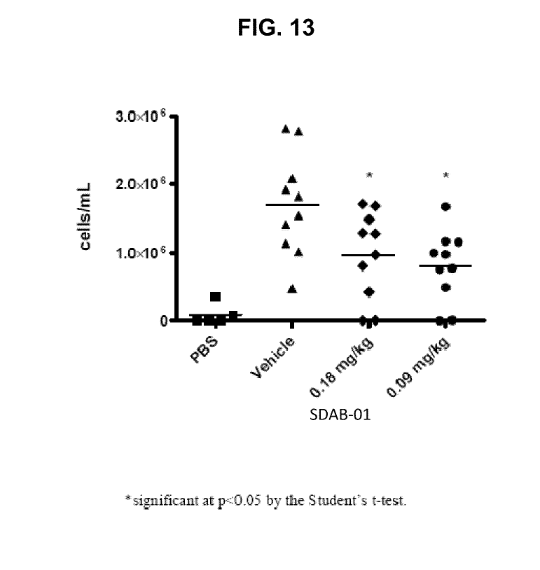

In experiment 3, SDAB-01 was administered at the same doses as previously performed. A significant decrease in total white blood cell infiltration was observed at the 0.09 mg/kg dose and neutrophil infiltration was observed with both doses of SDAB-01 as shown in FIG. 12 and FIG. 13. Lymphocytes were significantly decreased in the 0.09 mg/kg dose, but not the 0.18 mg/kg group. There was no effect on monocyte infiltration at any dose tested.

In summary, a significant inhibition of neutrophil infiltration was observed with both concentrations of SDAB-01 compared to the control group except in one study in which the 0.09 mg/kg dose gave a positive trend that was not significant (Table 4).

TABLE 4
Summary of Murine Air Pouch Experiments Using SDAB-01

| | Total WBC | | Neutrophils | | Lymphocytes | | Monocytes | |
|---|---|---|---|---|---|---|---|---|
| | 0.18 | 0.09 | 0.18 | 0.09 | 0.18 | 0.09 | 0.18 | 0.09 |
| Experiment | mg/kg | mg/kg | mg/kg | mg/kg | mg/kg | mg/kg | mg/kg | mg/kg |
| 1 | + | +/− | + | +/− | − | − | − | − |
| 2 | +/− | + | + | + | − | − | − | − |
| 3 | +/− | + | + | + | − | + | − | − |

+ significant decrease (p ≦ 0.05) in cellular infiltration compared to vehicle control.
+/− lower trend of infiltration, but not significant.
− no significant difference compared to vehicle control.

Administration of doses as low as 0.09 mg/kg of SDAB-01 significantly decreased cellular infiltration and neutrophil infiltration induced by 10 ng of recombinant human TNFα. There was little to no effect on lymphocyte and monocyte infiltration by any of the doses tested. These data indicate that SDAB-01 can consistently block the infiltration of neutrophils caused by recombinant human TNFα stimulation.

### Example 8

### Efficacy of SDAB-01 in Tg197 Human TNFα Transgenic Mouse Model of Arthritis

The therapeutic effect of SDAB-01 was assessed in the TNFα transgenic mouse model of rheumatoid arthritis. In this model, TNFα transgenic mice develop chronic polyarthritis with 100% incidence at 4-7 weeks of age. The disease is dependent on the over-expression of human TNFα. The effects of various treatment doses (10, 3, 1, 0.3, 0.1, 0.03 mg/kg) of SDAB-01 were studied in a therapeutic dosing regimen. Animals were randomly assigned to groups when 100% of the mice showed signs of disease. Once assigned to groups, treatment with SDAB-01, anti-TNFα Antibody control 2, control antibody or vehicle was initiated and continued twice weekly for 7 weeks. All animals were scored weekly in a blinded fashion for visual signs of disease symptoms. At the end of the study, hind paws were harvested, processed, and evaluated microscopically for indicators of disease.

In experiment 1, treatment with SDAB-01 at doses of 10, 3, and 1 mg/kg showed a significant effect by preventing further development of arthritis in a dose-dependent manner in comparison to the vehicle-treated group. Treatment with the higher doses of SDAB-01 (10, 3, 1 mg/kg) displayed significant amelioration of histopathological scores compared to both control groups. Therefore, the minimum therapeutic dose that showed an amelioration of arthritis compared to the control-treated groups assessed clinically and by microscope was 1 mg/kg SDAB-01.

In experiment 2, treatment with SDAB-01 at doses of 10, 3 and 1 mg/kg displayed a therapeutic effect on established arthritis with regression of both clinical and histopathological scores. Therefore, the minimum therapeutic dose that showed an amelioration of arthritis compared to the control-treated group assessed clinically and microscopically was 1 mg/kg.

In summary, anti-TNFα treatment with SDAB-01 displayed a dose dependent therapeutic effect on established arthritis, evidenced by prevention of disease exacerbation and regression of both clinical and histopathological scores. This treatment result was a direct consequence of specific antagonism towards human TNFα, since control antibody treatment recapitulated the pathology evident with vehicle treatment, in the Tg197 mouse arthritis model.

### Experimental Design

SDAB-01 and anti-tetanus toxin (control) antibody were prepared at Pfizer by standard methodologies. Infliximab (Remicade©, anti-TNFα antibody, Lot No. 7HD98016) was purchased from Med World Pharmacy (Catalog No. NDC 57894-030-01).

Male Tg197 mice, homozygous for the human TNF-globin hybrid transgene (maintained on a CBAxC57BL/6 genetic background) were crossed with (CBAxC57BL/6) F1 females. The heterozygous transgenic offspring were used in the studies. When 100% of the mice demonstrated signs of arthritis, all the mice were randomly assigned to treatment groups. On the day that the animals were assigned to a treatment group, the mice began receiving doses of PF-05230905, control antibody (anti-tetanus toxin antibody), Infliximab, or vehicle control (10 mM L-histidine, 5% sucrose buffer, Lot No. C-51683, D-20216) via intraperitoneal injections. The doses given and dosing frequency are described in each experiment's subsection. Both hind paws of each mouse were evaluated for the progression of disease at defined intervals as follows:
No arthritis, (normal appearance and flexion).
0.5 Onset of arthritis (mild joint swelling).
1 Mild arthritis (joint distortion).
1.5 As above with finger deformation, less strength on flexion.
2 Moderate arthritis (severe swelling, joint deformation, no strength on flexion).
2.5 As above with finger deformation in paws.
3 Heavy arthritis (ankylosis detected on flexion and severely impaired movement).

Each mouse was then assigned a mean score between 0-3. To monitor disease progression, 4 littermates of the Tg197 mice who also had arthritis were sacrificed at 6 weeks of age, at the treatment starting point. At the end of the studies, all mice were sacrificed and histopathological analysis of the ankle joints was performed. The scores from the experimental mice were compared to the 4 littermates. The histopathological score was evaluated in a blinded fashion microscopically from 0-4 as follows:
0 No detectable pathology
1 Hyperplasia of the synovial membrane and presence of polymorphonuclear infiltrates
2 Pannus and fibrous tissue formation and focal subchondrial bone erosion
3 Cartilage destruction and bone erosion
4 Extensive cartilage destruction and bone erosion.

### Experiment 1

In experiment 1, efficacy of various doses of SDAB-01 was evaluated in the therapeutic TNFα transgenic murine model of rheumatoid arthritis. The mice were monitored bi-weekly for signs of arthritis. When 100% of the mice showed signs of disease, all animals were randomly assigned to a treatment group. Heterozygous Tg197 mice were divided into groups of 8 mice each. Treatment began with SDAB-01 (10, 3, 1, 0.3, 0.1 mg/kg), control antibody (10 mg/kg), Infliximab (10, 3 mg/kg) or vehicle (Histidine/sucrose buffer, 10 mL/kg) bi-weekly. Treatment continued for 7 weeks and macroscopic changes in joint morphology (arthritic scores) and the average weight of each animal were recorded weekly. Following euthanasia with CO2, sera were harvested and two hind paws of each animal were processed for histological assessment.

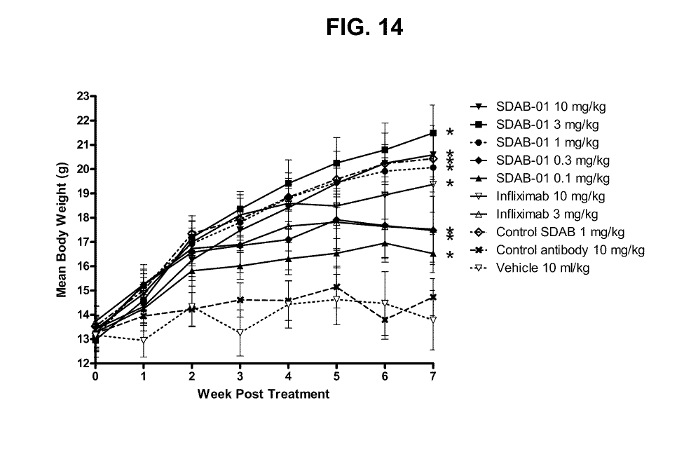

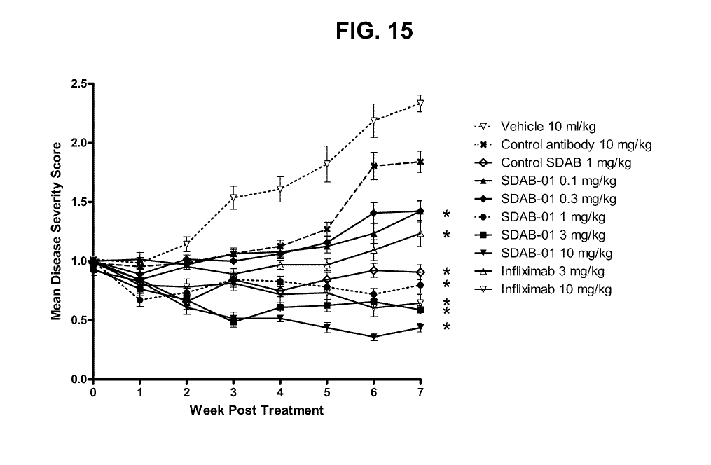

In experiment 1, administration of SDAB-01 showed a very significant effect by improving the body weight loss (FIG. 14) and preventing disease progression (FIG. 15) compared to the vehicle-treated group. Infliximab was identical to the SDAB-01 (10, 3, 1 mg/kg) doses in stabilizing the clinical scores.

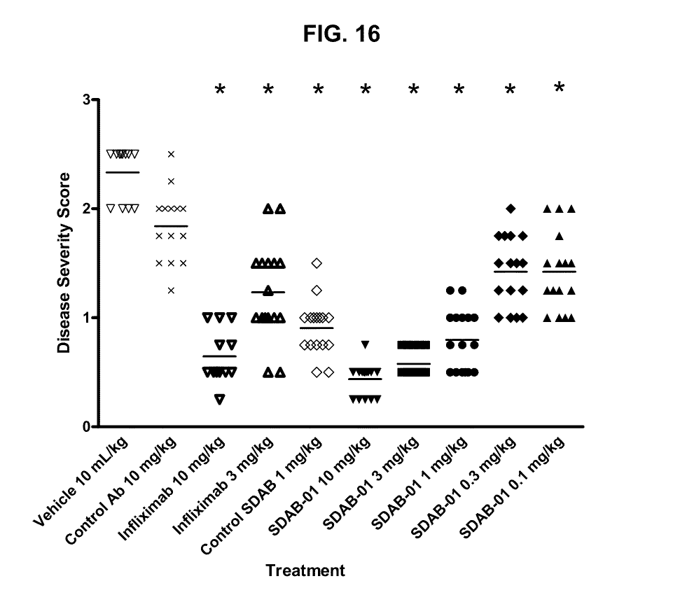

The severity of disease assessed on the last day of scoring is shown in FIG. 16. The number of animals with reduced disease symptoms was greatest in the groups that were treated with SDAB-01 (10, 3, 1 mg/kg) and Infliximab (10 mg/kg) in comparison with the vehicle or control antibody.

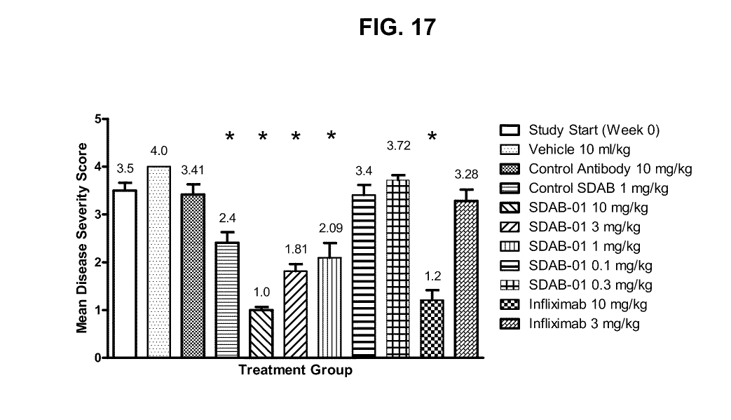

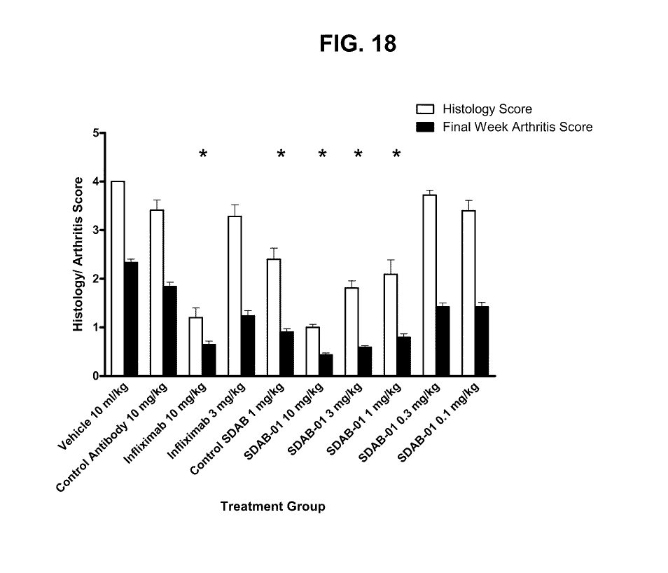

One hematoxylin and eosin-stained section from each of the two hind paws from each mouse was evaluated microscopically in a blinded fashion. Treatment with SDAB-01 (10, 3, 1 mg/kg) on established arthritis displayed efficacy by preventing disease exacerbation and gradually leading to regression of histopathological scores. This treatment result was a direct consequence of specific antagonism towards human TNFα, since control antibody treatment recapitulated the pathology evident with vehicle treatment (FIG. 17, FIG. 18).

### Experiment 2

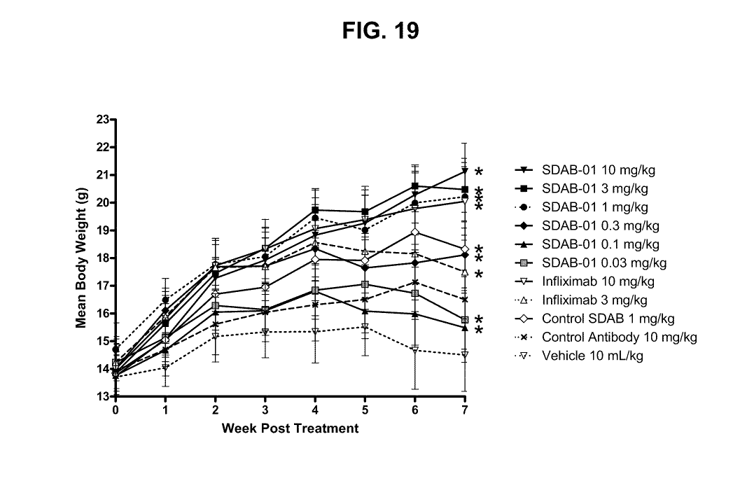

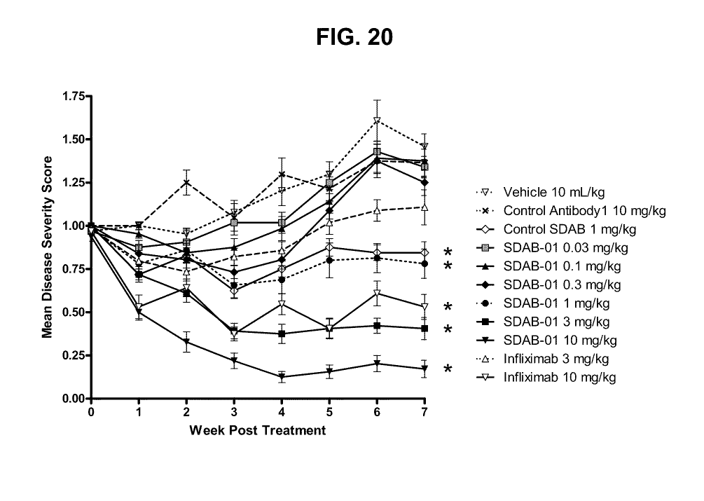

In experiment 2, the effect of treatment with SDAB-01 at 10, 3, 1, 0.3, and 0.1 mg/kg twice per week was repeated, and SDAB-01 at 0.03 mg/kg twice per week and Infliximab at 10 and 3 mg/kg twice per week were included. Doses of SDAB-01 (10, 3, 1, 0.3, 0.1, and 0.03) showed a significant effect by improving the body weight loss (FIG. 19). However, only doses of 10, 3, and 1 mg/kg were successful at preventing disease progression (FIG. 20) compared to the vehicle-treated group. Treatment with SDAB-01 (0.3 mg/kg), or Infliximab (3 mg/kg) resulted in a moderate, but not-significant improvement in clinical evaluation compared to either the vehicle- or control antibody-treated groups.

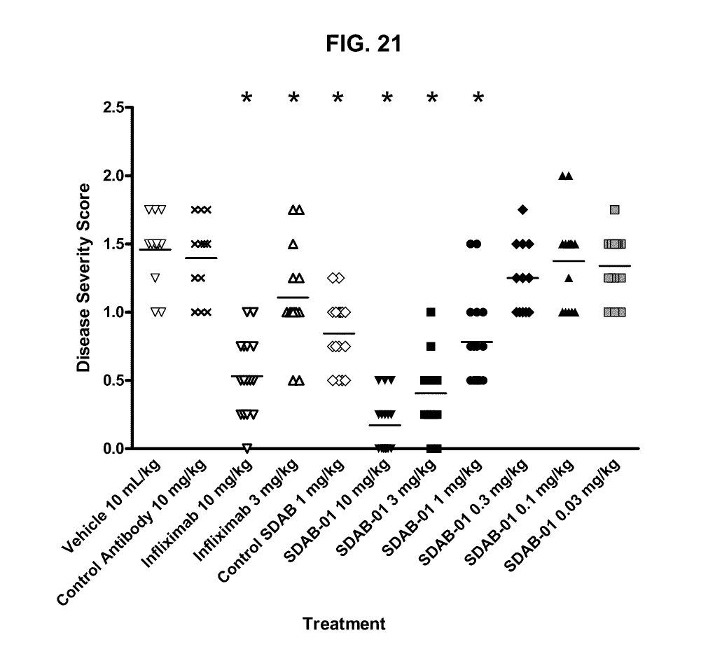

The severity of disease assessed on the last day of scoring is shown in FIG. 21. The number of animals with reduced disease symptoms was greatest in the groups that were treated with SDAB-01 (10, 3, 1 mg/kg) and Infliximab (10 mg/kg) in comparison with vehicle control.

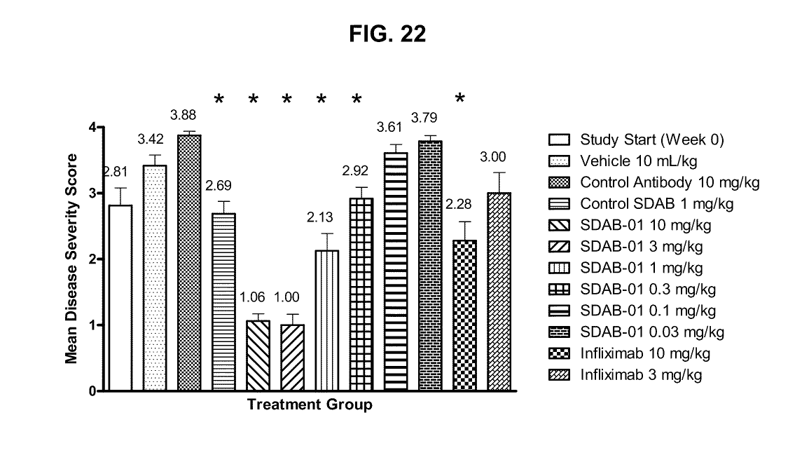

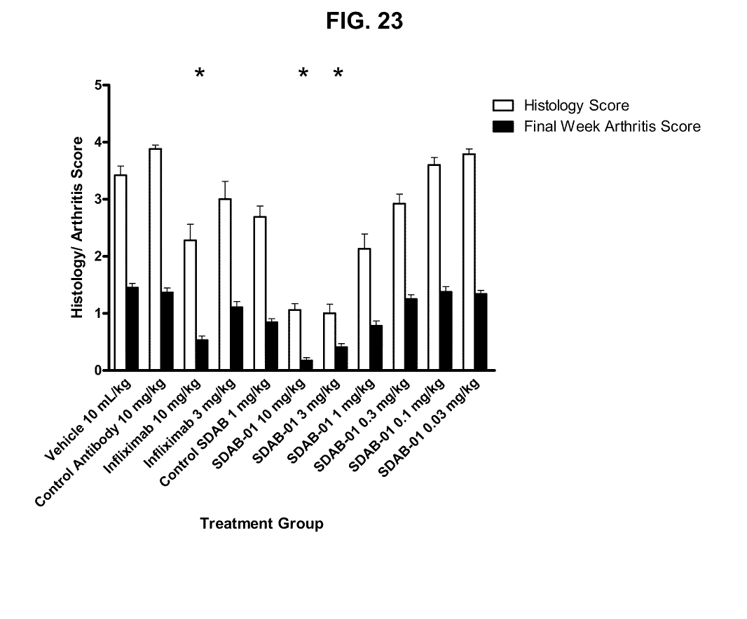

One hematoxylin and eosin-stained section from each of the two hind paws from each mouse was evaluated in a blinded fashion microscopically. Treatment with SDAB-01 (10, 3, 1 mg/kg) displayed efficacy on established arthritis by preventing disease exacerbation and gradually leading to regression of histopathological scores (FIG. 22). This treatment result was a direct consequence of specific antagonism towards human TNFα, since human control antibody treatment recapitulated the pathology evident with vehicle treatment. As the scores of the vehicle-treated group and the control antibody-treated group did not differ significantly, the 3 higher doses of SDAB-01 (10, 3, and 1 mg/kg) were effective as evidenced by a significant reduction in the histopathological scores compared to both control groups (FIG. 23). Hence the minimum effective dose assessed clinically and microscopically was 1 mg/kg SDAB-01.

Treatment with the 3 higher doses of SDAB-01 (10, 3 and 1 mg/kg) resulted in improved histopathological scores. These scores were significantly lower than the scores of the control littermates that were harvested at the start of the study.

At the MED of 1 mg/kg, the mean observed steady-state (end-bleed) serum SDAB-01 concentration was 4.81 μg/mL, which was within a 2-fold difference compared with the predicted steady-state (end-bleed) serum concentration of 7.70 μg/mL based on the pharmacokinetic profile of SDAB-01 after a single 1 mg/kg IP dose to Tg197 mice. The mean steady-state (end-bleed) serum SDAB-01 concentrations were 0.21, 42.1, and 120 μg/mL in the 0.3, 3, and 10 mg/kg dose groups, respectively. For the 0.03 and 0.1 mg/kg dose groups, all but one animal had serum SDAB-01 concentrations that were less than the limit of quantitation of 0.049 μg/mL.

### Example 9

### PEGylation of Bivalent SDAB Molecules Expressed in Pichia pastoris

Dithiotreitol (DTT) was added to the neutralized fractions to reduce potential disulfide bridges formed between the carboxy terminal cysteines of the SDAB molecules. A final concentration of 10 mM DTT and incubation overnight at 4° C. was found to be optimal. The reduction was evaluated by analytical size exclusion chromatography (SEC). Therefore 25 ml of the reduced SDAB molecule was added to 75 ml Dulbecco's PBS (D-PBS) and injected on a Sup75 10/300 GL column equilibrated in D-PBS.

Non-reduced SDAB molecule and DTT were removed by preparative SEC on a Hiload 26/60 Superdex 75 preparation grade column equilibrated in D-PBS.

The concentration of the reduced SDAB molecule was determined by measuring the absorbance at 280 nm. A Uvikon 943 Double Beam US/VIS spectrophotometer was used. The absorption was measured in a wavelength scan of 245-330 nm. Two precision cells made of Quartz Suprasil were used. First the absorption of the blank was measured at 280 nm by placing two cells filled by 900 μl D-PBS. The sample was diluted (1/10) by adding 100 μl of the sample to the first cell and mixing it before reading. The absorption of the sample was measured at 280 nm. The concentration was calculated with the following formula:

To PEGylate a SDAB molecule, a 5× molar excess of freshly made 1 mM PEG40 solution was added to the reduced SDAB molecule solution.

The SDAB molecule-PEG mixture was incubated for 1 hour at RT with gentle agitation and then transferred to 4° C. The PEGylation was evaluated via analytical SEC. Thereafter, 25 μl of the SDAB molecule was added to the 75 μl D-PBS and injected on a Sup75HR 10/300 column equilibrated in D-PBS. PEGylated SDAB molecule eluted in the range of the exclusion volume of the column (>75 KDa).

The PEGylated and non-PEGylated SDAB molecules were separated via cation exchange chromatography (CEX-Buffer A was 25 mM citric acid and Buffer B was 1M NaCl in PBS). The sample was diluted to a conductivity of 5 mS/cm and the pH was adjusted to 4.0. The column was equilibrated and after sample application was washed extensively with Buffer A. PEGylated SDAB molecule was eluted with a 3 CV gradient.

The collected SDAB molecule was buffer exchanged into D-PBS by SEC on a Hiload 26/60 Superdex 75 prep grade column equilibrated in D-PBS. The SDAB molecule was subsequently made LPS-free via passage over an anion exchange column. This column, was sanitized in 1M NaOH and afterwards equilibrated in endotoxin free D-PBS.

### Biotinylation

To biotinylate a SDAB molecule, a 5× molar excess of biotin from a 10 mM stock solution was added to the reduced SDAB molecule. The biotin SDAB molecule mixture was incubated for 1 h at RT with gentle agitation and then stored at 4° C.

The purity of biotinylated SDAB molecule was controlled via analytical SEC. 25 μl of biotinylated SDAB molecule was subsequently added to 75 μl of D-PBS and injected on a Sup75HR 10/300 column equilibrated in D-PBS. The resulting chromatogram showed that the SDAB molecule biotin needed no further purification: no dimerization of SDAB molecule via an oxidation of free sulfhydryls could be detected. A buffer change to D-PBS was done by a passage over a desalting column Sephadex G25 fine column.

The SDAB molecule-biotin was made LPS-free by passage over an anion exchange column. The column was sanitized overnight in 1M NaOH and then equilibrated in D-PBS.

### Example 10a

### Pharmacokinetics of SDAB-01 in Male Cynomolgous Monkeys Following Single Intravenous and Subcutaneous Administration

In the first study, SDAB-01 was administered by single IV or SC bolus injection to male cynomolgous monkeys (n=3 per group: monkeys SAN 1-3 for IV, monkeys SAN 4-6 for SC) at 3 mg/kg (based on protein content). Serum samples for PK analysis were collected from each animal prior to dosing (0 hour), and from 0.083 to 1536 hours post-dose. Additional serum samples were taken prior to dosing (0 hour) and at 336, 672, 1008, and 1536 hours post-dose to evaluate the formation of anti-SDAB-01 antibodies. The serum SDAB-01 concentrations were determined using a qualified enzyme-linked immunosorbent assay (ELISA) and the results were used to determine the pharmacokinetic parameters for SDAB-01. The presence of anti-SDAB-01 antibodies was determined using a qualified ELISA.

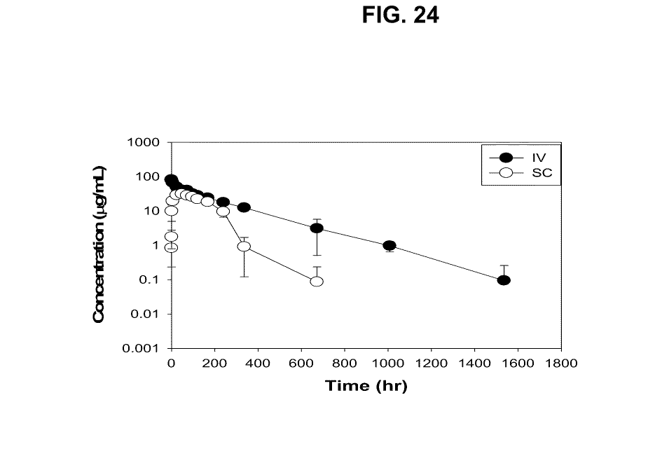

The mean serum concentration-time profiles of SDAB-01 in male cynomolgous monkeys after IV or SC administration are illustrated in FIG. 24. The mean pharmacokinetic parameters of SDAB-01 after IV or SC administration in monkeys are summarized in Table 5.

TABLE 5
Mean (±SD) Pharmacokinetic Parameters of SDAB-01 in Male
Cynomolgous Monkeys After Single IV or SC Administration of
3 mg/kg (Based on Protein Content, n = 3 per Treatment Group)

| | CL | Vdₛₛ | t_{1/2} | AUC_{0-∞} | Cₘₐₓ | Tₘₐₓ |
|---|---|---|---|---|---|---|
| Route | (mL/hr/kg) | (mL/kg) | (hr) | (μg · hr/mL) | (μg/mL) | (hr) |
| IV | 0.234 ± 0.028 | 51.5 ± 8.15 | 147 ± 78.4 | 12919 ± 1453 | 85.4^{a} ± 2.58 | NA |
| SC | NA | NA | 123 ± 11.5 | 8958 ± 526 | 31.7 ± 2.72 | 72 ± 0 |

^{a}Concentration at 5 min for SANs 1 and 3; Concentration at 0.5 hr for SAN 2 after IV administration.
NA. Not Applicable

SDAB-01 was absorbed well from the injection site after SC administration of 3 mg/kg. After a single SC dose of 3 mg/kg in three male cynomolgous monkeys, the mean maximum serum concentration (Cₘₐₓ) of 31.7±2.72 μg/mL was observed at 72 hours post-dosing, indicating that absorption of SDAB-01 after SC injection was a slow process. The terminal half-life ranged from 110 to 131 hours in three monkeys, with a mean value of 123 hours (approximately 5 days). The relatively short t_{1/2}, observed after SC administration might be due to the formation of anti-SDAB-01 antibodies.

Two monkeys from the SC treatment group positive for anti-SDAB-01 antibodies. The mean AUC from three monkeys was 8958 μg·hr/mL. The bioavailability after SC administration in monkeys cannot be accurately determined from this study due to the formation of anti-SDAB-01 antibody in both IV and SC treated monkeys. However, an estimate could be obtained by using the AUC_{0∞} ratio between SC and IV administration, which was found to be approximately 69.3%. This value should be used with caution since it may under- or over-estimate the bioavailability of SDAB-01 in monkeys.

Overall, anti-SDAB-01 antibody formation was detected in 50% (3/6) animals dosed with anti-SDAB-01. The incidence of anti-SDAB-01 antibodies was 33.3% (1/3) for animals in the 3 mg/kg IV group and 66.7% (2/3) for animals in the 3 mg/kg SC group. Antibodies (log titers 2.19-2.52) were detected at 1008 and 1536 hours after dosing for monkey SAN 1 (IV treatment group) and monkey SAN 5 (SC treatment group). Antibodies (log titer of 1.71) were detected at 1536 hours after dosing for monkey SAN 4 (SC treatment group). Because all pre-dose samples were negative, these animals were considered to have an immune response to SDAB-01. It should be noted that circulating levels of SDAB-01 may have interfered with the detection of anti-SDAB-01 antibodies.

The half-life of SDAB-01 was shorter in monkeys that were positive for anti-SDAB-01 antibody formation, suggesting that the formation of anti-SDAB-01 antibodies had an impact on the pharmacokinetics of SDAB-01 in monkeys.

In the second study, male and female cynomolgous monkeys (n=12 per group) were administered a single 5 mg/kg IV 100 mg/kg IV, and 100 mg/kg SC dose of SDAB-01 and serum concentration were measured using a qualified ELISA. After a 5 or 100 mg/kg IV dose of SDAB-01, the mean AUC_{0-∞}, CL, and t_{1/2} values were 24,600 and 395,000 μg·h/mL, 0.210 and 0.263, mL/hr/kg, and 149 and 144 hours, respectively. Systemic exposure (Cmax, AUC_{0∞}, and AUC₀₋₁₆₈) increased with increasing dose in an approximately dose-proportional manner. After a single 100 mg/kg SC dose, the mean Tmax, AUC_{0-∞}, and t_{1/2} values were 150 hours, 352,000 μg·h/mL, and 165 hours, respectively. The bioavailability after SC administration (estimated using mean AUC_{0-∞} values after 100 mg/kg IV and SC doses) was 89%. The incidence of anti-SDAB-01 antibodies was 4 of 12 (33.3%), 1 of 12 (8.3%), and 1 of 12 (8.3%) animals in the 5 mg/kg (IV), 100 mg/kg (IV), and 100 mg/kg (SC) dose groups, respectively.

### Example 10b

### Comparison of Serum Pharmacokinetics of SDAB-01 (TNFα SDAB Molecule 2×20 PEG), TNFα SDAB Molecule 4×10 PEG, and TNFα SDAB Molecule Linear 1×40 PEG

Serum PK profiles of TNFα SDAB molecule branched 2×20 kDa PEG, TNFα SDAB molecule branched 4×10 kDa PEG and TNFα SDAB molecule linear 1×40 kDa PEG constructs were examined in B6CBAF1/J mice, Sprague-Dawley rats and cynomolgous monkeys following a single IV administration of 2 or 3 mg/kg (based on protein content). Serum concentrations were determined using either specific ELISA (mice and monkeys) or gamma-counting (rats).

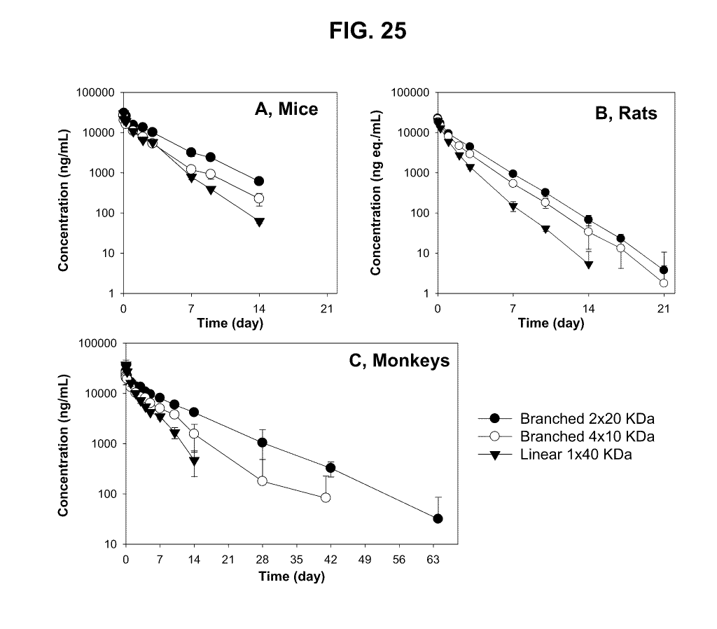

In all 3 species examined, the branched 2×20 kDa PEG construct had significantly higher exposure (AUC) compared to the linear 1×40 kDa PEG construct (p<0.05) (FIG. 25 and Tables 6-8). Specifically, the relative increase in mean dose-normalized AUC0-∞ for the branched 2×20 kDa PEG construct relative to the linear 1×40 kDa PEG construct was ˜94, 102, and 136% in mice, rats, and monkeys, respectively. Accordingly, the total body clearance (CL) of the branched 2×20 kDa PEG construct was lower and elimination half-life (t1/2) of the branched 2×20 kDa PEG appeared longer compared to the linear 1×40 kDa PEG construct. Specifically, the relative decrease in mean CL value for the branched 2×20 kDa PEG construct was ˜48, 50, and 66% in mice, rats, and monkeys, respectively and the relative increase in mean t1/2 values was 43, 26, 54% in mice, rats, and monkeys, respectively.

The branched 4×10 kDa PEG construct also had higher mean serum AUC0-∞ and lower CL, compared to the linear 1×40 kDa PEG construct in rats and monkeys, but not in mice (Tables 6-8). In rats and monkeys, the magnitude of change in PK parameters for the branched 4×10 kDa PEG construct relative to the linear construct were less pronounced (43-51% increase in AUC0-∞ and 35-45% decrease in CL), compared to those for the branched 2×20 kDa PEG construct.

TABLE 6
Pharmacokinetic parameters of PEGylated TNFα
SDAB molecules after a single IV dose to B6CBAF1/J mice.

| | | | AUC_{0-∞}/ | AUCₗₐₛₜ/ | | | |
|---|---|---|---|---|---|---|---|
| | Dose | C_{5 min} | Dose(μg · hr/ | Dose(μg · hr/ | CL | Vdₛₛ | t_{1/2} |
| Construct | (mg/kg) | (μg/mL) | mL)/(mg/kg) | mL)/(mg/kg) | (mL/hr/kg) | (mL/kg) | (hr) |
| TNFα SDAB | 2 | 55 | 2179 | 2121 ± 61 * | 0.46 | 40 | 66 |
| molecule | | | | | | | |
| branched 2x20 | | | | | | | |
| kDa PEG | | | | | | | |
| TNFα SDAB | 3 | 80 | 1193 | 1174 ± 23 | 0.84 | 58 | 56 |
| molecule | | | | | | | |
| branched 4x10 | | | | | | | |
| kDa PEG | | | | | | | |
| TNFα SDAB | 3 | 89 | 1126 | 1122 ± 22 | 0.89 | 46 | 46 |
| molecule | | | | | | | |
| linear 1x40 | | | | | | | |
| kDa PEG | | | | | | | |

Male B6CBAF1/J mice were administered a single IV bolus dose of the indicated test article. Serum samples were taken at 5 min to 14 days post dose once from each mouse (n = 3 per time point), and serum concentrations were determined by the specific ELISA. PK parameters were determined by non-compartmental analysis using the sparse sampling method and statistical analysis of AUClast/Dose values was performed using ANOVA with Dunnett's post test, with the linear 1x40 PEG group as the control.
Star (*) indicates statistically significant differences (p < 0.05) relative to the linear PEG group.
C5 min = Concentration at 5 min, the first sampling time point after IV administration.
CL = Total body clearance based on serum concentration.
Vdss = Volume of distribution at steady-state.
t½ = Elimination half-life.
AUC0-∞ = Area under the concentration-time curve from time 0 to infinity.
AUClast = Area under the concentration-time curve from time 0 up to sampling time at which a quantifiable concentration is found.

TABLE 7
Pharmacokinetic parameters (mean ± SD) of 125I-labeled PEGylated TNFα
SDAB molecules after a single IV dose to Sprague-Dawley rats

| | | | | AUC_{0-∞}/ | | | |
|---|---|---|---|---|---|---|---|
| | Dose | C_{5 min} | AUC_{0-∞} | Dose (μg eq. · hr/ | CL | Vdₛₛ | t_{1/2} |
| Compound | (mg/kg) | (μg eq./mL) | (μg eq. · hr/mL) | mL)/(mg/kg) | (mL/hr/kg) | (mL/kg) | (hr) |
| TNFα SDAB | 2 | 46 ± 4.9 | 2025 ± 214 | 1013 ± 107 | 1.0 ± 0.12 * | 53 ± 5.5 * | 44 ± 3.5 |
| molecule | | | | | | | |
| branched 2x20 | | | | | | | |
| kDa PEG | | | | | | | |
| TNFα SDAB | 2 | 44 ± 2.2 | 1514 ± 78 | 757 ± 39 | 1.3 ± 0.07 * | 63 ± 4.1 | 42 ± 8.2 |
| molecule | | | | | | | |
| branched 4x10 | | | | | | | |
| kDa PEG | | | | | | | |
| TNFα SDAB | 2 | 39 ± 2.6 | 1001 ± 62 | 500 ± 31 | 2.0 ± 0.13 | 65 ± 2.2 | 35 ± 5.7 |
| molecule | | | | | | | |
| linear 1x40 | | | | | | | |
| kDa PEG | | | | | | | |

Male Sprague-Dawley rats were given a single IV bolus dose of the indicated 125I-labeled test article, serum samples were taken at 5 min to 24 days post dose, and radioactive equivalent (RE) concentrations in serum were determined by gamma-counting. PK parameters were calculated for each individual animal (n = 7 for 2X20 and 4x10 kDa PEG constructs and n = 5 for the 1x40 kDa PEG construct) by non-compartmental analysis. Statistical analyses of AUC0-∞, AUC0-∞/Dose, CL, Vdss, and t½ values were performed using ANOVA with Dunnett's post test, with the linear 1x40 kDa PEG group as the control.
Star (*) indicates statistically significant differences relative to the linear PEG (p < 0.05).

TABLE 8
Pharmacokinetic parameters (mean ± SD) of PEGylated TNFα
SDAB molecules after a single IV dose to cynomolgous monkeys

| | | | | AUC_{0-∞}/ | | | |
|---|---|---|---|---|---|---|---|
| | Dose | C_{5 min} | AUC_{0-∞} | Dose (μg · hr/ | CL | Vdₛₛ | t_{1/2} |
| Construct | (mg/kg) | (μg/mL) | (μg · hr/mL) | mL)/(mg/kg) | (mL/hr/kg) | (mL/kg) | (hr) |
| TNFα SDAB | 3 | 82 ± 6.4 | 13293 ± 820 * | 4431 ± 273 * | 0.23 ± 0.01 * | 57 ± 4.8 * | 188 ± 19 * |
| molecule | | | | | | | |
| branched 2x20 | | | | | | | |
| kDa PEG | | | | | | | |
| TNFα SDAB | 3 | 76 ± 3.2 | 8055 ± 736 * | 2685 ± 245 * | 0.37 ± 0.03 * | 79 ± 12 | 153 ± 30 * |
| molecule | | | | | | | |
| branched 4x10 | | | | | | | |
| kDa PEG | | | | | | | |
| TNFα SDAB | 3 | 111 ± 26 | 5637 ± 263 | 1879 ± 88 | 0.67 ± 0.10 | 78 ± 13 | 122 ± 12 |
| molecule | | | | | | | |
| linear 1x40 | | | | | | | |
| kDa PEG | | | | | | | |

Male cynomolgous monkeys were administered a single IV bolus dose of the indicated test article, serum samples were taken at 5 min to 62, 57, and 56 days for the 2x20, 4x10, and 1x40 kDa PEG constructs, respectively, and serum concentrations were determined by ELISA. PK parameters were calculated for each individual animal (n = 3 per construct) by non-compartmental analysis. Data points with the sharp concentration drop were not used for PK calculations (for one of the 3 monkey dosed with the 2x20 kDa PEG construct). Statistical analyses of AUC_{0-∞}, AUC_{0-∞}/Dose, CL, Vdₛₛ, and t_{1/2} values were performed using ANOVA with Dunnett's post test, with the linear 1x40 kDa PEG group as the control.
Star (*) indicates statistically significant differences relative to the linear PEG group (p < 0.05).

Additional studies were performed for SDAB-01 construct only:

First, mouse and monkey serum samples were analyzed using two different immunoassay formats: immunoassay that measures whole molecule versus protein portion of the molecule. The Protein Detection Assay captured the PEGylated drug conjugate through the protein portion by utilizing a biotinylated target molecule. The polyclonal anti-drug antibody detector also bound the protein portion of the molecule, and thus assay detected free and PEGylated protein. The whole molecule assay detection assay used the same capture mode as the Protein Detection assay, but detection occurred through the PEG moiety via a monoclonal rabbit anti-PEG antibody. This detector antibody is specific for the methoxy group of the PEG molecule. The assay format did not significantly impact PK profiles and calculated parameters in mouse and monkey animal models

Second, pharmacokinetic profiles of SDAB-01 were examined after a single SC or IP dose to mice. After a single 2 mg/kg SC dose or 3 mg/kg IP dose to male B6CBAF1/J mice, the Tmax was 24 hours; the t1/2 values were 52.4 hours (approximately 2.2 days) and 57.7 hours (approximately 2.4 days), respectively. The bioavailability after IP or SC administration was 68.7% and 56.6%, respectively. After a single 0.3 mg/kg IP dose to male Tg197 mice, the Tmax, t1/2, and AUC0-∞ values were 6 hours, 24.6 hours, and 165 μg·h/mL, respectively. Increase in the IP dose to 1 mg/kg, resulted in an approximately dose-proportional increase in exposure (AUC0-∞=528 μg·h/mL), with Tmax (6 hours) and t1/2 (21.4 hours) values comparable to those observed at 0.3 mg/kg.

### Example 10c

### Biodistribution of SDAB-01 (TNFα SDAB Molecule 2×20 PEG) and TNFα SDAB Molecule LINEAR 1×40 PEG

Biodistribution of TNFα SDAB molecule branched 2×20 kDa PEG and TNFα SDAB molecule linear 1×40 kDa PEG constructs were examined over 7 days (168 hr) in B6CBAF1/J mice following a single IV dose of 0.3 mg/kg (based on protein content) of 1251-labeled test articles. Radioactive equivalent (RE) serum and tissue concentrations were determined using gamma-counting, serum and tissue exposures (AUC0-168 hr) and tissue-to-serum (T/S) AUC ratios were calculated.

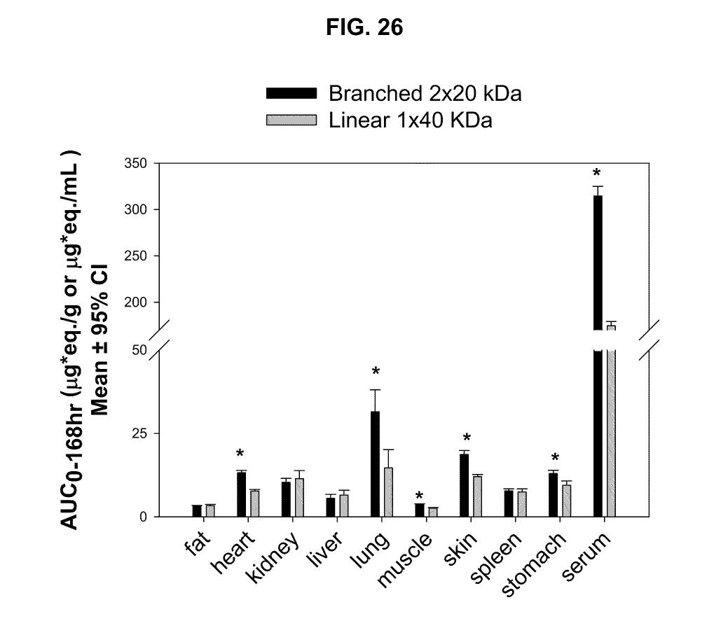

Similar to the observation in the earlier study in B6CBAF1/J mice with non-radiolabeled PEG conjugates, the branched 2×20 kDa PEG construct had ˜80% higher AUC0-168 hr (p<0.05), compared to the linear 1×40 kDa construct (FIG. 26). The branched construct also had significantly higher exposures in some but not all tissues examined (FIG. 26). Specifically, the increase in AUC0-168 hr for the branched 2×20 kDa PEG construct relative to the linear 1×40 kDa PEG construct was 72, 115, 43, 55, and 80% in heart, lung, muscle, skin, and stomach, respectively. T/S AUC ratios (Table 9) and T/S concentration ratios (data not shown) were approximately similar between the two constructs for these tissues.

In contrast to serum, heart, lung, muscle, skin, and stomach, the AUC0-168 hr in fat, kidney, liver, and spleen were similar for the two constructs, leading to lower T/S AUC ratios (Table 9) and T/S concentration ratios (data not shown) for the branched 2×20 kDa PEG construct.

For both TNFα SDAB molecule Linear 1×40 PEG and SDAB-01, approximately 60% of total administered radioactivity was excreted in urine within 1 week (168 hours) after dosing, with most of the radioactivity excreted in urine (approximately 70%) attributed to free iodine.

TABLE 9
Tissue-to-serum (T/S) AUC ratios of ¹²⁵I-labeled PEGylated TNFα
Nanobodies after a single 0.3 mg/kg IV dose to B6CBAF1/J mice

| | TNFα | TNFα |
|---|---|---|
| | Nanobody ™_branched | Nanobody ™_linear |
| Tissue | 2 × 20 kDa PEG | 1 × 40 kDa PEG |
| fat | 0.01 | 0.02 |
| heart | 0.04 | 0.04 |
| kidney | 0.03 | 0.07 |
| liver | 0.02 | 0.04 |
| lung | 0.10 | 0.08 |
| muscle | 0.01 | 0.01 |
| skin | 0.06 | 0.07 |
| spleen | 0.02 | 0.04 |
| stomach | 0.04 | 0.05 |

B6CBAF1/J mice were administered a single 0.3 mg/kg IV bolus dose of ¹²⁵I-labeled TNFα SDAB molecule branched 2 × 20 kDa PEG (black bars) or TNFα SDAB molecule linear 40 kDa PEG (gray bars). Serum and tissue samples (n = 8 − 12 per time point) were collected over the 7 days (168 hr) and radioactive equivalent (RE) concentrations in tissue and serum were determined by gamma-counting, as described in the text. AUC_{0-168 hr} for serum (in μg × eq./mL) and each tissue (μg × eq./g) were determined by non-compartmental analysis using the sparse sampling method and the tissue-to-serum (T/S) AUC ratios (AUC_{0-168 hr, tissue}/AUC_{0-168 hr, serum}) was calculated.

### Example 11

### Biophysical Analyses of SDAB Molecules and Control Molecules

To investigate potential reasons for differential PK profiles of three TNFα SDAB molecule 40 kDa PEG conjugates, additional biophysical analyses were conducted.

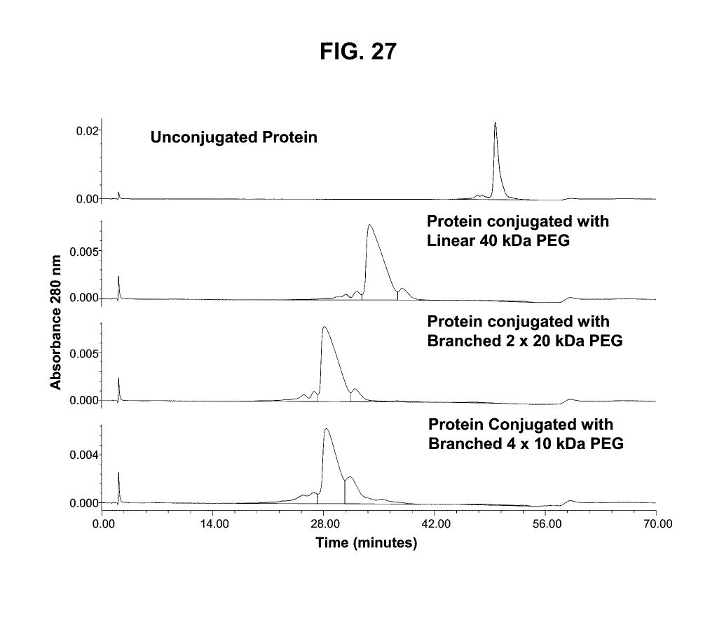

The CEX-HPLC was performed to monitor the charge heterogeneity of the three constructs. The representative chromatographic profiles are presented in FIG. 27. A significant amount of charge heterogeneity was observed for all PEGylated TNFα SDAB molecule conjugates. The main peak of the linear PEG conjugate eluted at a later retention time when compared to the two branched conjugates (2×20 kDa and 4×10 kDa), suggesting the linear conjugate has more exposed positive charges on the surface compared to the branched conjugates. The retention time of the main peak for the two branched conjugates (2×20 kDa and 4×10 kDa) was similar. By comparison, the unconjugated protein eluted much later than all of the PEGylated conjugates tested, indicating it has even greater positive surfaces charge density. The theoretical isoelectric point of the unconjugated protein is greater than 9; therefore the protein is predicted to have a net positive charge in the CEX running buffers, which are at pH 4.0.

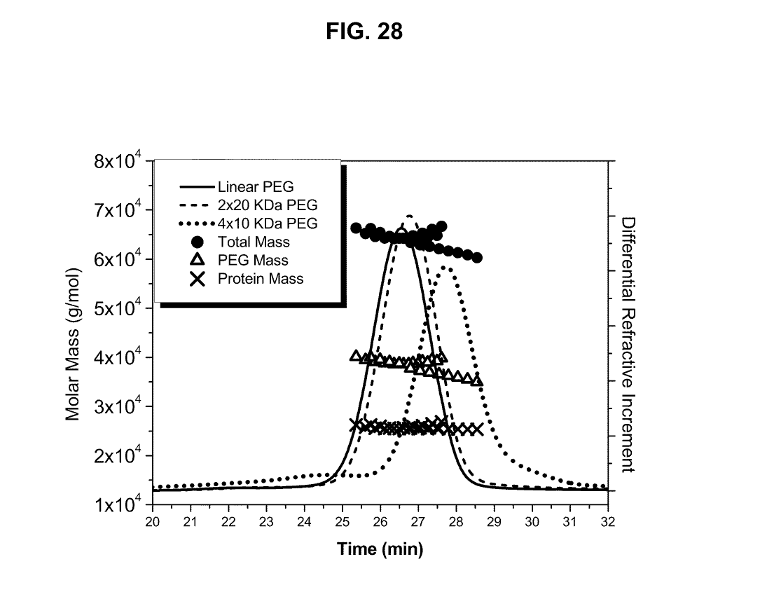

Size and mass distributions were determined using SE-HPLC with Multi-Angle Light Scattering (MALS) monitored by UV absorbance, differential refractometry (dRI), and on-line quasi-elastic light-scattering (QELS). Since the PEG on the TNFα SDAB molecule-PEG conjugates does not absorb at 280 nm, it is possible to determine the distributions of protein and PEG in the conjugate using SEC-MALS with UV and dRI detection. The calculated protein and PEG mass distributions were consistent with each other for all 3 conjugates (Table 10 and FIG. 28).

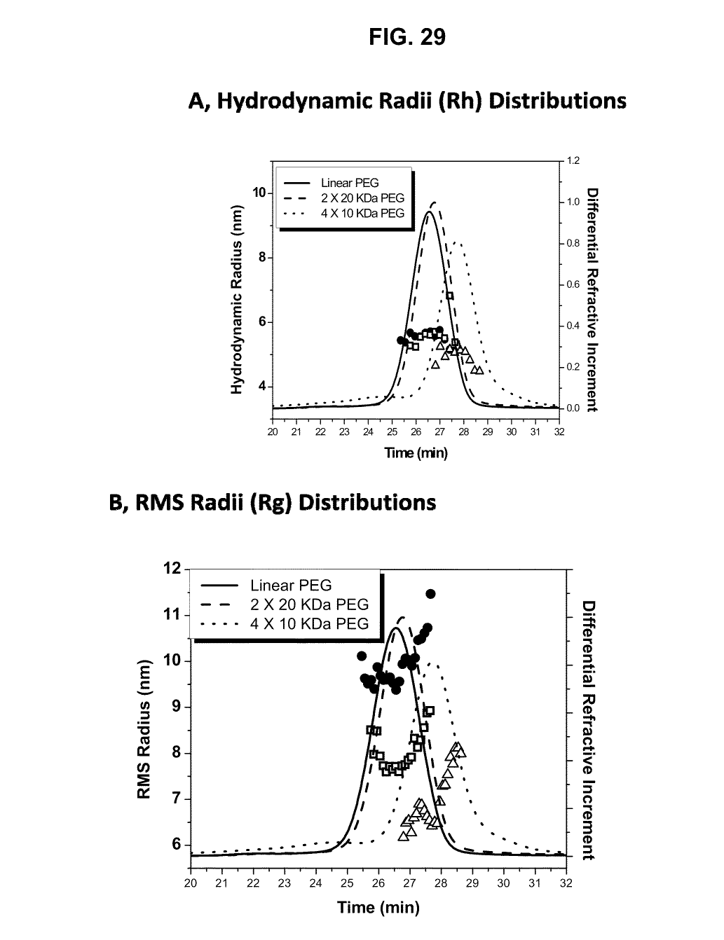

The branched 4×10 kDa PEG conjugate had a noticeably later elution volume on the SEC-MALS than the branched 2×20 kDa and linear 1×40 kDa PEG conjugates, indicating that the branched 4×10 kDa PEG conjugate is hydrodynamically smaller compared to the other two conjugates (FIG. 29). The smaller hydrodynamic radius (Rh, defined as the radius of a sphere with the same diffusion coefficient as the sample being measured) of the 4×10 kDa branched PEG conjugate was confirmed by QELS measurements (Table 10).

Using the angular dependency of the scattered light measured by MALS, the distribution of root mean squared (RMS) radii can be determined. The RMS radius (also referred to as radius of gyration, Rg) is a measurement of the root mean square distance that all parts of the molecule are from the center of its mass at any given time and provides information about the average volume a molecule occupies. Both the branched 2×20 kDa and the branched 4×10 kDa PEG conjugates had smaller Rg (RMS radii) than the linear PEG conjugate (Table 10 and FIG. 29).

Finally, conformational information can be obtained by calculating the RMS/Rh(Rg/Rh) ratio: the larger the value of the ratio, the more elongated or extended the molecule is. The RMS/R^{h} ratio was 1.77, 1.45, and 1.37 for the linear 1×40 kDa PEG, branched 2×20 kDa, and branched 4×10 kDa PEG conjugates, respectively, indicating that the conjugate with the linear 1×40 kDa PEG had a more extended conformation than the more compact conjugates containing the branched PEGs (Table 10). It should be noted that SE-HPLC method used to analyze the PEGylated conjugates is not suitable for side-by-side analysis of the unconjugated proteins.

TABLE 10
Calculated Weight Averaged Mass and Sizes of PEGylated
TNFα SDAB molecule from SEC-MALS Analysis

| | Total | PEG | Protein | | | |
|---|---|---|---|---|---|---|
| | Molar | Molar | Molar | RMS(Rg) | Rh | |
| | Mass | Mass | Mass | Radius | Radius | RMS/ |
| | (kDa) | (kDa) | (kDa) | (nm) | (nm) | Rh |
| TNFα SDAB | 64.86 | 39.01 | 25.85 | 9.9 | 5.6 | 1.77 |
| molecule | | | | | | |
| linear 1x40 | | | | | | |
| kDa PEG | | | | | | |
| TNFα SDAB | 64.54 | 38.97 | 25.57 | 8 | 5.5 | 1.45 |
| molecule | | | | | | |
| branched 2x20 | | | | | | |
| kDa PEG | | | | | | |
| TNFα SDAB | 61.78 | 36.29 | 25.49 | 7 | 5.1 | 1.37 |
| molecule | | | | | | |
| branched 4x10 | | | | | | |
| kDa PEG | | | | | | |

All samples were diluted to 2.0 mg/mL and 100 μL of each sample was injected over a Superose 6 column (400 mM NaCl. 20 mM NaPO₄, pH 7.2 at 0.5 mL/min) held at 30° C. Molar masses, Rh, and RMS were determined using ASTRA V v5.3.4.14 from Wyatt Technologies.

All three SDAB PEG conjugates and the unPEGylated protein had ≧92% bioactivity, relative to the PEGylated reference material in the cell-based bioassay (based on TNFα induced apoptosis in U937 cells), suggesting that the PEGylation did not alter the activity of the protein.

TABLE 11
Protein sequences

| | SEQ ID | |
|---|---|---|
| Name | NO | Sequence |
| GS9 | 12 | GGGGSGGGS |
| GS30 | 13 | GGGGSGGGGSGGGGSGGGGSGGGGSGGGGS |
| TNF1-GS9- | 14 | QVQLVESGGGLVQPGGSLRLSCAASGFTFSDYWMYWVRQAPGKGLE |
| TNF1 | | WVSE1NTNGLITKYPDSVKGRFTISRDNAKNTLYLQMNSLKPEDTA |
| (TNF4 ) | | LYYCARSPSGFNRGQGTQVTVSSGGGGSGGGSQVQLVESGGGLVQP |
| | | GGSLRLSCAASGFTFSDYWMYWVRQAPGKGLEWVSEINTNGLITKY |
| | | PDSVKGRFTISRDNAKNTLYLQMNSLKPEDTALYYCARSPSGFNRG |
| | | QGTQVTVSS |
| TNF2-GS9- | 15 | QVQLVESGGGLVQAGGSLRLSCAASGRTFSEPSGYTYTIGWFRQAP |
| TNF2 (TNF5) | | GKEREFVARIYWSSGLTYYADSVKGRFTISRDIAKNTVDLLMNSLK |
| | | PEDTAVYYCAARDGIPTSRSVGSYNYWGQGTQVTVSSGGGGSGGGS |
| | | QVQLVESGGGLVQAGGSLRLSCAASGRTFSEPSGYTYTIGWFRQAP |
| | | GKEREFVARIYWSSGLTYYADSVKGRFTISRDIAKNTVDLLMNSLK |
| | | PEDTAVYYCAARDGIPTSRSVGSYNYWGQGTQVTVSS |
| TNF3-GS9- | 16 | EVQLVESGGGLVQAGGSLSLSCSASGRSLSNYYMGWFRQAPGKERE |
| TNF3 (TNF6) | | LLGNISWRGYN1YYKDSVKGRFTISRDDAKNTIYLQMNRLKPEDTA |
| | | VYYCAASILPLSDDPGWNTYWGQGTQVTVSSGGGGSGGGSEVQLVE |
| | | SGGGLVQAGGSLSLSCSASGRSLSNYYMGWFRQAPGKERELLGNIS |
| | | WRGYNIYYKDSVKGRFTISRDDAKNTIYLQMNRLKPEDTAVYYCAA |
| | | SILPLSDDPGWNTYWGQGTQVTVSS |
| TNF1-GS30- | 17 | QVQLVESGGGLVQPGGSLRLSCAASGFTFSDYWMYWVRQAPGKGLE |
| TNF1 (TNF7) | | WVSEINTNGLITKYPDSVKGRFTISRDNAKNTLYLQMNSLKPEDTA |
| | | LYYCARSPSGFNRGQGTQVTVSSGGGGSGGGGSGGGGSGGGGSGGG |
| | | GSGGGGSQVQLVESGGGLVQPGGSLRLSCAASGFTFSDYWMYWVRQ |
| | | APGKGLEWVSE1NTNGLITKYPDSVKGRFTISRDNAKNTLYLQMNS |
| | | LKPEDTALYYCARSPSGFNRGQGTQVTVSS |
| TNF2-GS30- | 18 | QVQLVESGGGLVQAGGSLRLSCAASGRTFSEPSGYTYTIGWFRQAP |
| TNF2 (TNF8) | | GKEREFVARIYWSSGLTYYADSVKGRFTISRD1AKNTVDLLMNSLK |
| | | PEDTAVYYCAARDGIPTSRSVGSYNYWGQGTQVTVSSGGGGSGGGG |
| | | SGGGGSGGGGSGGGGSGGGGSQVQLVESGGGLVQAGGSLRLSCAAS |
| | | GRTFSEPSGYTYTIGWFRQAPGKEREFVARIYWSSGLTYYADSVKG |
| | | RFTISRDIAKNTVDLLMNSLKPEDTAVYYCAARDGIPTSRSVGSYN |
| | | YWGQGTQVTVSS |
| TNF3-GS30- | 19 | EVQLVESGGGLVQAGGSLSLSCSASGRSLSNYYMGWFRQAPGKERE |
| TNF3 (TNF9) | | LLGNISWRGYNIYYKDSVKGRFTISRDDAKNTIYLQMNRLKPEDTA |
| | | VYYCAASILPLSDDPGWNTYWGQGTQVTVSSGGGGSGGGGSGGGGS |
| | | GGGGSGGGGSGGGGSEVQLVESGGGLVQAGGSLSLSCSASGRSLSN |
| | | YYMGWFRQAPGKERELLGNISWRGYN1YYKDSVKGRFTISRDDAKN |
| | | TIYLQMNRLKPEDTAVYYCAASILPLSDDPGWNTYWGQGTQVTVSS |
| TNF30-30GS- | 11 | EVQLVESGGGLVQPGGSLRLSCAASGFTFSDYWMYWVRQAPGKGLE |
| TNF30-C | | WVSEINTNGLITKYPDSVKGRFTISRDNAKNTLYLQMNSLRPEDTA |
| (TNF55) | | VYYCARSPSGFNRGQGTLVTVSSGGGGSGGGGSGGGGSGGGGSGGG |
| | | GSGGGGSEVQLVESGGGLVQPGGSLRLSCAASGFTFSDYWMYWVRQ |
| | | APGKGLEWVSEINTNGLITKYPDSVKGRFTISRDNAKNTLYLQMNS |
| | | LRPEDTAVYYCARSPSGFNRGQGTLVTVSC |
| TNF30-30GS- | 1 | EVQLVESGGGLVQPGGSLRLSCAASGFTFSDYWMYWVRQAPGKGLE |
| TNF30-gggC | | WVSEINTNGLITKYPDSVKGRFTISRDNAKNTLYLQMNSLRPEDTA |
| (TNF56) | | VYYCARSPSGFNRGQGTLVTVSSGGGGSGGGGSGGGGSGGGGSGGG |
| | | GSGGGGSEVQLVESGGGLVQPGGSLRLSCAASGFTFSDYWMYWVRQ |
| | | APGKGLEWVSEINTNGLITKYPDSVKGRFTISRDNAKNTLYLQMNS |
| | | LRPEDTAVYYCARSPSGFNRGQGTLVTVSSgggC |

TABLE 12
cDNA sequences

| | SEQ | |
|---|---|---|
| | ID | |
| Name | NO | Sequence |
| TNF30- | 5 | Atgagatttccttcaatttttactgctgttttattcgcagcatcctccgcattagc |
| 30GS- | | tgctccagtcaacactacaacagaagatgaaacggcacaaattccggctgaagctg |
| TNF30-C | | tcatcggttactcagatttagaaggggatttcgatgttgctgttttgccattttcc |
| (TNF55) | | aacagcacaaataacgggttattgtttataaatactactattgccagcattgctgc |
| | | taaagaagaaggggtatctctcgagaaaagagaggtgcagctggtggagtctggtg |
| | | gaggcttggttcaaccgggtggcagcctgcgtttatcctgcgcagcctctggtttc |
| | | acctttagtgattactggatgtattgggttcgtcaggctccagggaaaggcctcga |
| | | atgggtgtcggaaattaatactaatggtcttatcacaaaatacccggacagcgtta |
| | | agggccgtttcaccatctcccgcgataacgctaaaaacacgctgtatctgcaaatg |
| | | aacagcctgcgtcctgaagacacggccgtatattactgtgcgcgctctccgagcgg |
| | | ttttaaccgcggccaggggacccttgtcaccgtctcctcaggcggtggaggcagcg |
| | | gtggcgggggtagcggcggtggaggcagcggtggcgggggatccggcggtggaggc |
| | | agcggtggcgggggtagcgaggtgcagctggtggagtctggtggaggcttggttca |
| | | accgggtggcagcctgcgtttatcctgcgcagcctctggtttcacctttagtgatt |
| | | actggatgtattgggttcgtcaggctccagggaaaggcctcgaatgggtgtcggaa |
| | | attaatactaatggtcttatcacaaaatacccggacagcgttaagggccgtttcac |
| | | catctcccgcgataacgctaaaaacacgctgtatctgcaaatgaacagcctgcgtc |
| | | ctgaagacacggccgtatattactgtgcgcgctctccgagcggttttaaccgcggc |
| | | caggggacccttgttaccgtctcctgctaataa |
| TNF30- | 6 | atgagatttccttcaatttttactgctgttttattcgcagcatcctccgcattagc |
| 30GS- | | tgctccagtcaacactacaacagaagatgaaacggcacaaattccggctgaagctg |
| TNF30- | | tcatcggttactcagatttagaaggggatttcgatgttgctgttttgccattttcc |
| gggC | | aacagcacaaataacgggttattgtttataaatactactattgccagcattgctgc |
| (TNF56) | | taaagaagaaggggtatctctcgagaaaagagaggtgcagctggtggagtctggtg |
| | | gaggcttggttcaaccgggtggcagcctgcgtttatcctgcgcagcctctggtttc |
| | | acctttagtgattactggatgtattgggttcgtcaggctccagggaaaggcctcga |
| | | atgggtgtcggaaattaatactaatggtcttatcacaaaatacccggacagcgtta |
| | | agggccgtttcaccatctcccgcgataacgctaaaaacacgctgtatctgcaaatg |
| | | aacagcctgcgtcctgaagacacggccgtatattactgtgcgcgctctccgagcgg |
| | | ttttaaccgcggccaggggacccttgtcaccgtctcctcaggcggtggaggcagcg |
| | | gtggcgggggtagcggcggtggaggcagcggtggcgggggatccggcggtggaggc |
| | | agcggtggcgggggtagcgaggtgcagctggtggagtctggtggaggcttggttca |
| | | accgggtggcagcctgcgtttatcctgcgcagcctctggtttcacctttagtgatt |
| | | actggatgtattgggttcgtcaggctccagggaaaggcctcgaatgggtgtcggaa |
| | | attaatactaatggtcttatcacaaaatacccggacagcgttaagggccgtttcac |
| | | catctcccgcgataacgctaaaaacacgctgtatctgcaaatgaacagcctgcgtc |
| | | ctgaagacacggccgtatattactgtgcgcgctctccgagcggttttaaccgcggc |
| | | caggggacccttgtcaccgtctcctcaggtggaggttgctaataa |

### EQUIVALENTS

All references cited herein are incorporated herein by reference in their entirety and for all purposes to the same extent as if each individual publication or patent or patent application was specifically and individually indicated to be incorporated by reference in its entirety for all purposes.

The present invention is not to be limited in scope by the specific embodiments described herein. Indeed, various modifications featured in the invention, in addition to those described herein, will become apparent to those skilled in the art from the foregoing description and accompanying figures. Such modifications are intended to fall within the scope of the appended claims.

We claim:

1. A modified single domain antigen binding molecule, comprising:
(i) one or more single antigen binding domains that bind to one or more targets;
(ii) a non-peptidic linker; and
(iii) one or more polymer molecules,
wherein the non-peptidic linker is a moiety of formula (I): 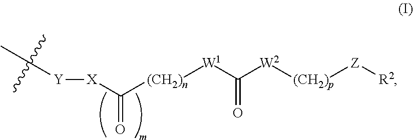
wherein
W¹ and W² are each independently selected from a bond or NR¹;
Y is a bond, C₁₋₄ alkylene substituted with 0-2 occurrences of R^{a} or a pyrrolidine-2,5-dione;
X is O, a bond or is absent;
Z is O, NR³, S or a bond;
R¹ and R³ are each independently hydrogen or C₁₋₆ alkyl;
R² is absent or is one or more polymer moieties.
R^{a} is selected from hydroxyl, C₁₋₄ alkyl or C₁₋₄ alkoxy;
m is 0 or 1;
n is 0, 1, 2 or 3;
p is 0, 1, 2, 3 or 4.

2. The modified single domain antigen binding molecule of claim 1, wherein the one or more polymer molecules comprise a poly(ethyleneglycol (PEG) monomer or a derivative thereof.

3. The modified single domain antigen binding molecule of claim 2, wherein the PEG polymer molecule is branched and the PEG monomer is methoxypoly(ethyleneglycol) (mPEG) or a derivative thereof.

4. The modified single domain antigen binding molecule of claim 3, wherein the PEG polymer molecule is a branched PEG polymer molecule selected from the group consisting of formulas (a)-(h):

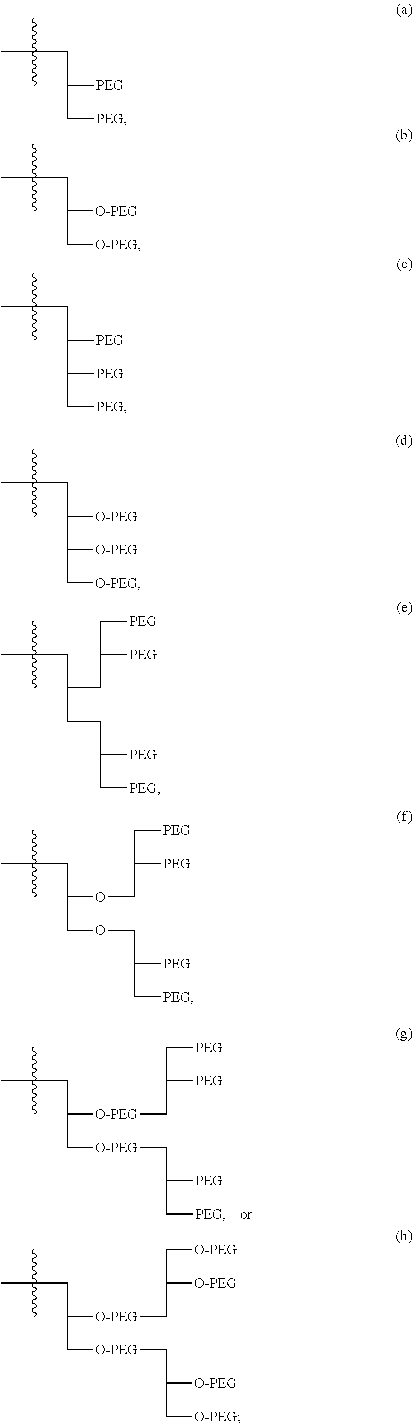

5. The modified single domain antigen binding molecule of claim 2, wherein each PEG polymer moiety independently has a molecular weight between 1 KDa and 100 KDa.

6. The modified single domain antigen binding molecule of claim 5, wherein each PEG polymer moiety independently has a molecular weight between 10 KDa and 50 KDa.

7. The modified single domain antigen binding molecule of claim 5, wherein each PEG polymer moiety independently has a molecular weight selected from the group consisting of 10 KDa, 20 KDa, 30 KDa, 40 KDa and 50 KDa.

8. The modified single domain antigen binding molecule of claim 5, wherein the linker and the PEG polymer molecule have a structure selected from the group consisting of:

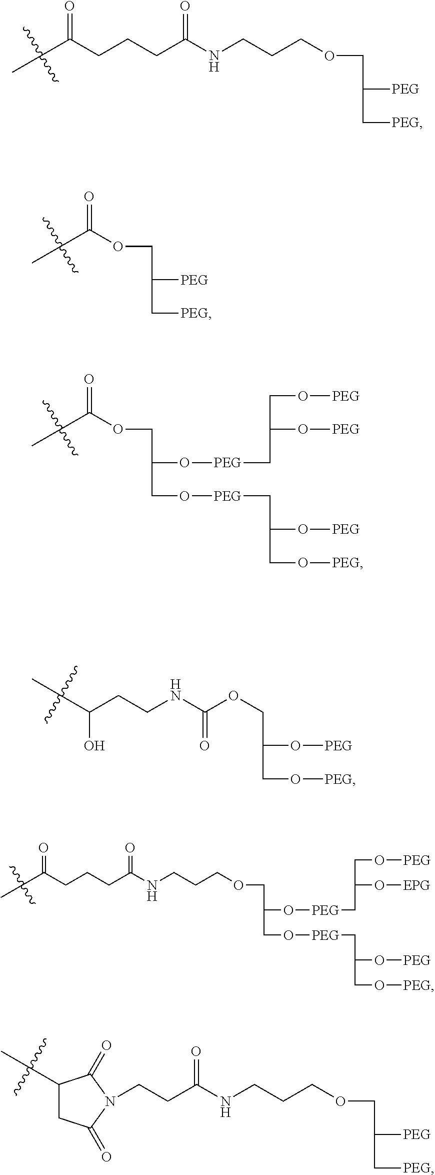

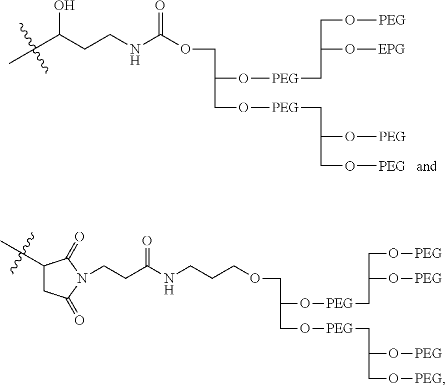

9. The modified single domain antigen binding molecule of claim 8, wherein the linker and the PEG polymer molecule are represented by the following formula:

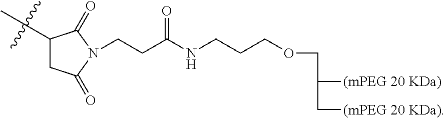

10. The modified single domain antigen binding molecule of claim 1, wherein at least one of said single antigen binding domains binds to human TNFα.

11. The modified single domain antigen binding molecule of claim 1, which is monovalent, bivalent or trivalent.

12. The modified single domain antigen binding molecule of claim 1, which is monospecific, bispecific, or trispecific.

13. The modified single domain antigen binding molecule of claim 1, wherein one or more of said single antigen binding domains is CDR-grafted, humanized, camelized, de-immunized, or selected by phage display.

14. The modified single domain antigen binding molecule of claim 1, which is a single chain fusion polypeptide comprising in the following order from N- to C-terminus: Anti-TNFα single antigen binding domain—(optionally, a peptidic linker)—anti-TNFα single antigen binding domain—non-peptidic linker—one or more polymer molecules.

15. The modified single domain antigen binding molecule of claim 1, wherein the one or more of said single antigen binding domains comprise the amino acid sequence shown in FIG. 2 or an amino acid sequence at least 85% identical thereto.

16. The modified single domain antigen binding molecule of claim 14, wherein the one or more of said single antigen binding domains comprise three CDRs having the amino sequence: DYWMY (CDR1), EINTNGLITKYPDSVKG (CDR2) and SPSGFN (CDR3), or having a CDR that differs by 1 amino acid substitution from one of said CDRs.

17. The modified single domain antigen binding molecule of claim 14, wherein said peptidic linker comprises at least one, two, three, four, five, six, seven or more repeats of (Gly)₃-Ser or (Gly)₄-Ser (SEQ ID NO:8).

18. The modified single domain antigen binding molecule of claim 17, which is represented by the following structure:

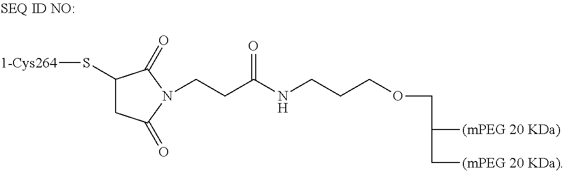

19. A pharmaceutical composition comprising the modified single domain antigen binding molecule according to claim 1 and a pharmaceutically acceptable carrier.

20. The pharmaceutical composition of claim 19, further comprising a second agent chosen from one or more of a cytokine inhibitor, a growth factor inhibitor, an immunosuppressant, an anti-inflammatory agent, a metabolic inhibitor, an enzyme inhibitor, a cytotoxic agent, or a cytostatic agent.

21. A method of ameliorating an inflammatory or an autoimmune condition in a subject, comprising administering to the subject the modified single domain antigen binding molecule according to claim 1, in an amount such that one or more of the symptoms of the TNFα associated disorder are reduced.

22. The method of claim 21, further comprising administering a second agent in combination with the modified single domain antigen binding molecule, wherein said second agent is chosen from one or more of a cytokine inhibitor, a growth factor inhibitor, an immunosuppressant, an anti-inflammatory agent, a metabolic inhibitor, an enzyme inhibitor, a cytotoxic agent, or a cytostatic agent.

23. The method of claim 21, wherein the TNFα-associated disorder is chosen from one or more of rheumatoid arthritis (RA), arthritic conditions, psoriatic arthritis, polyarticular juvenile idiopathic arthritis (JIA), ankylosing spondylitis (AS), psoriasis, ulcerative colitis, Crohn's disease, inflammatory bowel disease, or multiple sclerosis.

24. The method of claim 23, wherein the modified single domain antigen binding molecule or the second agent is administered to the subject by subcutaneous, intravascular, intramuscular or intraperitoneal injection or by inhalation.

25. A method of evaluating a modified single domain antigen binding molecule of claim 1, comprising:
administering the modified SDAB molecule according to claim 1 to a subject; and
evaluating one or more pharmacokinetic/pharmacodynamic (PK/PD) parameters of the modified SDAB molecule.

26. A method of evaluating or selecting a modified single domain antigen binding molecule of claim 1, comprising:
providing a test value for at least one PK/PD parameter of the modified SDAB molecule according to claim 1 to a subject; in a subject; and
comparing the test value provided with at least one reference value, to thereby evaluate or select the modified SDAB molecule.

27. The method of claim 25, further comprising: providing a sample containing the modified SDAB molecule; and testing the sample in a capture detection assay.

28. The method according to claim 25, wherein the PK/PD parameter evaluated is chosen from one or more of: an in vivo concentration of the modified SDAB molecule (e.g., a concentration in blood, serum, plasma and/or tissue); clearance of the modified SDAB molecule (CL); steady-volume distribution of the modified SDAB molecule (V_{dss}); half-life of the modified SDAB molecule (t_{1/2}); bioavailability of the modified SDAB molecule; dose normalized maximum blood, serum or plasma concentration of the modified SDAB molecule; dose normalized exposure of the modified SDAB molecule; or tissue-to-serum ratio of the modified SDAB molecule.

29. A capture detection assay for evaluating a modified single domain binding molecule of claim 1, comprising: providing a target immobilized to a solid support; and a reagent that binds to the protein or polymer moiety of the modified single domain antigen binding molecule for detecting the bound modified single domain antigen binding molecule-target complex.

30. A kit or an article of manufacture that includes a device, a syringe or a vial containing the modified single domain binding molecule of claim 1, and, optionally, including instructions for use.

31. A method of making a modified single domain binding molecule of claim 1 comprising:
providing a single domain binding molecule;
contacting the single domain binding molecule with a non-peptidic linker of formula (I): 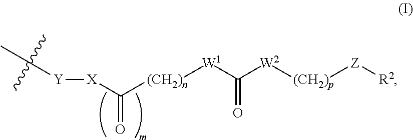
wherein
W¹ and W² are each independently selected from a bond or NR¹;
Y is a bond, C₁₋₄ alkylene substituted with 0-2 occurrences of R^{a} or a pyrrolidine-2,5-dione;
X is O, a bond or is absent;
Z is O, NR³, S or a bond;
R¹ and R³ are each independently hydrogen or C₁₋₆ alkyl;
R² is absent or is one or more polymer moieties.
R^{a} is selected from hydroxyl, C₁₋₄ alkyl or C₁₋₄ alkoxy;
m is 0 or 1;
n is 0, 1, 2 or 3;
p is 0, 1, 2, 3 or 4,
under conditions where at least one chemical bond is formed.


SEQUENCE LISTING (19 sequences)

SEQ ID NO: 1 - protein, 264 aa, Artificial Sequence
  SDAB-01
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSGGGGSGGGGSGGGGSGGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGC

SEQ ID NO: 2 - protein, 5 aa, Artificial Sequence
  CDR1
DYWMY

SEQ ID NO: 3 - protein, 17 aa, Artificial Sequence
  CDR2
EINTNGLITKYPDSVKG

SEQ ID NO: 4 - protein, 6 aa, Artificial Sequence
  CDR3
SPSGFN

SEQ ID NO: 5 - DNA, 1041 bp, Artificial Sequence
  TNF55 nucleotide
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggtgcagctggtggagtctggtggaggcttggttcaaccgggtggcagcctgcgtttatcctgcgcagcctctggtttcacctttagtgattactggatgtattgggttcgtcaggctccagggaaaggcctcgaatgggtgtcggaaattaatactaatggtcttatcacaaaatacccggacagcgttaagggccgtttcaccatctcccgcgataacgctaaaaacacgctgtatctgcaaatgaacagcctgcgtcctgaagacacggccgtatattactgtgcgcgctctccgagcggttttaaccgcggccaggggacccttgtcaccgtctcctcaggcggtggaggcagcggtggcgggggtagcggcggtggaggcagcggtggcgggggatccggcggtggaggcagcggtggcgggggtagcgaggtgcagctggtggagtctggtggaggcttggttcaaccgggtggcagcctgcgtttatcctgcgcagcctctggtttcacctttagtgattactggatgtattgggttcgtcaggctccagggaaaggcctcgaatgggtgtcggaaattaatactaatggtcttatcacaaaatacccggacagcgttaagggccgtttcaccatctcccgcgataacgctaaaaacacgctgtatctgcaaatgaacagcctgcgtcctgaagacacggccgtatattactgtgcgcgctctccgagcggttttaaccgcggccaggggacccttgttaccgtctcctgctaataa

SEQ ID NO: 6 - DNA, 1053 bp, Artificial Sequence
  TNF56 nucleotide
atgagatttccttcaatttttactgctgttttattcgcagcatcctccgcattagctgctccagtcaacactacaacagaagatgaaacggcacaaattccggctgaagctgtcatcggttactcagatttagaaggggatttcgatgttgctgttttgccattttccaacagcacaaataacgggttattgtttataaatactactattgccagcattgctgctaaagaagaaggggtatctctcgagaaaagagaggtgcagctggtggagtctggtggaggcttggttcaaccgggtggcagcctgcgtttatcctgcgcagcctctggtttcacctttagtgattactggatgtattgggttcgtcaggctccagggaaaggcctcgaatgggtgtcggaaattaatactaatggtcttatcacaaaatacccggacagcgttaagggccgtttcaccatctcccgcgataacgctaaaaacacgctgtatctgcaaatgaacagcctgcgtcctgaagacacggccgtatattactgtgcgcgctctccgagcggttttaaccgcggccaggggacccttgtcaccgtctcctcaggcggtggaggcagcggtggcgggggtagcggcggtggaggcagcggtggcgggggatccggcggtggaggcagcggtggcgggggtagcgaggtgcagctggtggagtctggtggaggcttggttcaaccgggtggcagcctgcgtttatcctgcgcagcctctggtttcacctttagtgattactggatgtattgggttcgtcaggctccagggaaaggcctcgaatgggtgtcggaaattaatactaatggtcttatcacaaaatacccggacagcgttaagggccgtttcaccatctcccgcgataacgctaaaaacacgctgtatctgcaaatgaacagcctgcgtcctgaagacacggccgtatattactgtgcgcgctctccgagcggttttaaccgcggccaggggacccttgtcaccgtctcctcaggtggaggttgctaataa

SEQ ID NO: 7 - protein, 4 aa, Artificial Sequence
  linker 1
GGGS

SEQ ID NO: 8 - protein, 5 aa, Artificial Sequence
  linker 2
GGGGS

SEQ ID NO: 9 - protein, 9 aa, Artificial Sequence
  LINKER 3
GGGGSGGGS

SEQ ID NO: 10 - protein, 5 aa, Artificial Sequence
  LINKER 4
GGGGS

SEQ ID NO: 11 - protein, 9 aa, Artificial Sequence
  LINKER 5
GGGGSGGGS

SEQ ID NO: 12 - protein, 30 aa, Artificial Sequence
  LINKER 6
GGGGSGGGGSGGGGSGGGGSGGGGSGGGGS

SEQ ID NO: 13 - protein, 238 aa, Artificial Sequence
  TNF4
QVQLVESGGGLVQPGGSLRLSCAASGFTFSDYWMYWVRQAPGKGLEWVSENTNGLITKYPDSVKGRFTISRDNAKNTLYLQMNSLKPEDTALYYCARSPSGFNRGQGTQVTVSSGGGGSGGGSQVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSS

SEQ ID NO: 14 - protein, 267 aa, Artificial Sequence
  TNF5
QVQLVESGGGLVQAGGSLRLSCAASGRTFSEPSGYTYTIGWFRQAPGKEREFVARIYWSSGLTYYADSVKGRFTISRDIAKNTVDLLMNSLKPEDTAVYYCAARDGIPTSRSVGSYNYWGQGTQVTVSSGGGGSGGGSQVQLVESGGGLVQAGGSLRLSCAASGRTFSEPSGYTYTIGWFRQAPGKEREFVARIYWSSGLTYYADSVKGRFTISRDIAKNTVDLLMNSLKPEDTAVYYCAARDGIPTSRSVGSYNYWGQGTQVTVSS

SEQ ID NO: 15 - protein, 254 aa, Artificial Sequence
  TNF6
EVQLVESGGGLVQAGGSLSLSCSASGRSLSNYYMGWFRQAPGKERELLGNISWRGYNYYKDSVKGRFTISRDDAKNTIYLQMNRLKPEDTAVYYCAASILPLSDDPGWNTYWGQGTQVTVSSGGGGSGGGSEVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSS

SEQ ID NO: 16 - protein, 259 aa, Artificial Sequence
  TNF7
QVQLVESGGGLVQPGGSLRLSCAASGFTFSDYWMYWVRQAPGKGLEWVSEINTNGLITKYPDSVKGRFTISRDNAKNTLYLQMNSLKPEDTALYYCARSPSGFNRGQGTQVTVSSGGGGSGGGGSGGGGSGGGGSGGGGSGGGGSQVQLVESGGGLVQPGGSLRLSCAASGFTFSDYWMYWVRQAPGKGLEWVSENTNGLITKYPDSVKGRFTISRDNAKNTLYLQMNSLKPEDTALYYCARSPSGFNRGQGTQVTVSS

SEQ ID NO: 17 - protein, 287 aa, Artificial Sequence
  TNF8
QVQLVESGGGLVQAGGSLRLSCAASGRTFSEPSGYTYTIGWFRQAPGKEREFVARIYWSSGLTYYADSVKGRFTISRDAKNTVDLLMNSLKPEDTAVYYCAARDGIPTSRSVGSYNYWGQGTQVTVSSGGGGSGGGGSGGGGSGGGGSGGGGSGGGGSQVQLVESGGGLVQAGGSLRLSCAASGRTFSEPSGYTYTIGWFRQAPGKEREFVARIYWSSGLTYYADSVKGRFTISRDIAKNTVDLLMNSLKPEDTAVYYCAARDGIPTSRSVGSYNYWGQGTQVTVSS

SEQ ID NO: 18 - protein, 275 aa, Artificial Sequence
  TNF9
EVQLVESGGGLVQAGGSLSLSCSASGRSLSNYYMGWFRQAPGKERELLGNISWRGYNIYYKDSVKGRFTISRDDAKNTIYLQMNRLKPEDTAVYYCAASILPLSDDPGWNTYWGQGTQVTVSSGGGGSGGGGSGGGGSGGGGSGGGGSGGGGSEVQLVESGGGLVQAGGSLSLSCSASGRSLSNYYMGWFRQAPGKERELLGNISWRGYNYYKDSVKGRFTISRDDAKNTIYLQMNRLKPEDTAVYYCAASILPLSDDPGWNTYWGQGTQVTVSS

SEQ ID NO: 19 - protein, 260 aa, Artificial Sequence
  TNF55
EVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSSGGGGSGGGGSGGGGSGGGGSGGGGSGGGGSEVQLVESGGGLVQPGGSLRLSCAASGFTFSDYWMYWVRQAPGKGLEWVSEINTNGLITKYPDSVKGRFTISRDNAKNTLYLQMNSLRPEDTAVYYCARSPSGFNRGQGTLVTVSC